SEQUENCE LISTING (36 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0190932 A1
(43) Pub. Date: Jun. 22, 2023

(54) METHODS FOR TREATMENT OF RELAPSED/REFRACTORY FOLLICULAR LYMPHOMA WITH MOSUNETUZUMAB AND LENALIDOMIDE

(71) Applicants: Genentech, Inc., South San Francisco, CA (US); Hoffmann-La Roche Inc., Little Falls, NJ (US)

(72) Inventors: Catherine GRANIER, Welwyn Garden City (GB); Andrea Knapp, Muttenz BL (CH); Chi-Chung Li, South San Francisco, CA (US); Carol Elaine O'Hear, South San Francisco, CA (US); Enkhtsetseg Purev, South San Francisco, CA (US); Michael C. Wei, South San Francisco, CA (US)

(21) Appl. No.: 18/050,736

(22) Filed: Oct. 28, 2022

Related U.S. Application Data
(60) Provisional application No. 63/273,689, filed on Oct. 29, 2021.

(51) Int. Cl. A61K 39/395; A61K 31/454; A61P 35/00; A61P 7/00

(52) U.S. Cl. CPC A61K 39/3955; A61K 31/454; A61P 35/00; A61P 7/00; A61K 2039/545

(57) ABSTRACT

The present invention relates to the treatment of subjects having relapsed and/or refractory (R/R) follicular lymphoma (FL). More specifically, the invention pertains to the treatment of subjects having R/R FL by administering a combination of mosunetuzumab and lenalidomide.

### CROSS-REFERENCE TO RELATED APPLICATIONS

This application claims benefit of priority to U.S. Provisional Application No. 63/273,689, filed on Oct. 29, 2021, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Sep. 9, 2022, is named 50474-262002_Sequence_Listing_9_9_22 and is 34,003 bytes in size.

### FIELD OF THE INVENTION

The present invention relates to the treatment of subjects having relapsed and/or refractory (R/R) follicular lymphoma (FL). More specifically, the invention pertains to combination treatment of subjects having R/R FL, who may have received prior therapy with an anti-CD20 antibody (e.g., an anti-CD20 monoclonal antibody, e.g., rituximab or obinutuzumab) or other treatment, by administration of mosunetuzumab and lenalidomide.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Indolent cancers can also severely effect quality of life. Cancer care thus represents a significant and ever-increasing societal burden.

B cell proliferative disorders are a leading cause of cancer-related deaths. For example, non-Hodgkin's lymphoma (NHL) advances quickly and is fatal if untreated. In the United States, B-cell lymphomas constitute approximately 80%-85% of all cases of NHL. Diffuse large B-cell lymphoma (DLBCL) is the most common type of NHL accounting for approximately 30%-40% of all NHL diagnosis, followed by follicular lymphoma (FL; 20%-25% of all NHL diagnosis) and mantle cell lymphoma (MCL; 6%-10% of all NHL diagnosis). B-cell chronic lymphocytic leukemia (CLL) is the most common leukemia in adults, with approximately 15,000 new cases per year in the United States (American Cancer Society 2015).

Lymphomas of B-cell origin constitute a diverse set of neoplasms within the larger context of non-Hodgkin lymphoma (NHL). Follicular lymphoma (FL) is the most common subtype of indolent NHL (Al-Hamandi et al. 2015). Regardless of the biologic and clinical heterogeneity of B-cell lymphomas, patients with advanced-stage B-cell malignancies are typically treated, initially, with intensive cytotoxic chemotherapy combined with monoclonal antibodies (mAbs) such as the anti-CD20 MAb, rituximab (RITUXAN®, MABTHERA®). Although durable responses can be achieved in some patients, the majority of patients will ultimately experience progressive or relapsed disease. FL remains an incurable disease with currently available therapies. The addition of rituximab to commonly used induction chemotherapy, including cyclophosphamide, doxorubicin, vincristine, and prednisone (CHOP); cyclophosphamide, vincristine, and prednisone (CVP); fludarabine; or bendamustine (Zelenetz et al. 2014, Forstpointer et al. 2006), followed by rituximab maintenance therapy led to prolonged remission and improved patient outcomes. However, treatment with said therapies only result in partial, but not complete, responses in the majority of patients with relapsed or refractory (R/R) FL. As such, multiply relapsed FL remains a disease with a high, unmet medical need for which improved therapies are needed.

### SUMMARY OF THE INVENTION

The present invention relates to methods of treating a subject having a relapsed and/or refractory (R/R) follicular lymphoma (FL) by administration of mosunetuzumab and lenalidomide as a combination therapy. In particular, the present invention relates to methods of treating a subject having a R/R FL that is R/R to at least one prior systemic therapy which included an anti-CD20 antibody (e.g. an anti-CD20 monoclonal antibody, e.g., rituximab or obinutuzumab) or other treatment.

In one aspect, the invention features a method of treating a subject, comprising administering to the subject an effective amount of mosunetuzumab and an effective amount of lenalidomide, wherein the subject: (a) has relapsed or refractory follicular lymphoma (R/R FL) that expresses CD20, and (b) has been previously treated with at least one chemo-immunotherapy regimen (e.g., R-CHP (rituximab, cyclophosphamide, doxorubicin, and prednisone), R-CHOP (rituximab, cyclophosphamide, doxorubicin, vincristine, and prednisone), CVP (cyclophosphamide, vincristine, and prednisone), or BR (bendamustine and rituximab)) (e.g., at least one chemo-immunotherapy that included an anti-CD20 monoclonal antibody (e.g., rituximab or obinutuzumab)). In some embodiments, the subject has received only one prior line of systemic therapy and either: (a) has a Follicular Lymphoma International Prognostic Index (FLIPI; Solal-Céligny et al. Blood. 2004; 104 (5): 1258-1265.) score of 2-5 (e.g., a score of 2, 3, 4, or 5), (b) has been refractory to prior anti-CD20 monoclonal antibody treatment, or (c) has disease progression within 24 months after initiation of prior therapy. In some embodiments, the subject has not been treated with the anti-CD20 monoclonal antibody for at least 4 weeks (e.g., 4 weeks, 6 weeks, 8 weeks, 10 weeks, 12 weeks, 24 weeks, 36 weeks, 48 weeks, 60 weeks, or more) prior to being administered the effective amount of mosunetuzumab and lenalidomide. In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments the synergistic effect is a complete response, as defined by Lugano 2014 criteria (Cheson B D, et al. J Clin Oncol 2014; 32:1-9).

In some embodiments, administering the effective amount of mosunetuzumab comprises administering mosunetuzumab according to a dosing regimen comprising at least a first dosing cycle and a second dosing cycle, wherein: (a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg (e.g., 0.05 mg to 4.0 mg, 0.1 mg to 4.0 mg, 0.2 mg to 4.0 mg, 0.3 mg to 4.0 mg, 0.4 mg to 4.0 mg, 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.1 mg to 3.5 mg, 0.1 mg to 3.0 mg, 0.1 mg to 2.5 mg, 0.1 mg to 2.0 mg, 0.1 mg to 1.5 mg, 0.1 mg to 1 mg, 0.1 mg to 0.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 0.05 mg to 20.0 mg (e.g., 0.1 mg to 20.0 mg, 0.2 mg to 20.0 mg, 0.3 mg to 20.0 mg, 0.4 mg to 20.0 mg, 0.5 mg to 20.0 mg, 1.0 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 0.5 mg to 15.0 mg, 0.5 mg to 10.0 mg, 0.5 mg to 9.0 mg, 0.5 mg to 8.0 mg, 0.5 mg to 7.0 mg, 0.5 mg to 6.0 mg, 0.5 mg to 5.0 mg, 0.5 mg to 4.0 mg, 0.5 mg to 3.0 mg, 0.5 mg to 2.0 mg, 0.5 mg to 1.0 mg, 1.0 mg to 15.0 mg, 1.0 mg to 10.0 mg, 1.0 mg to 8.0 mg, 1.0 mg to 6.0 mg, 1.0 mg to 4.0 mg, 1.0 mg to 3.0 mg, 10.0 mg to 20.0 mg, 15.0 mg to 20.0 mg, 5.0 mg to 15.0 mg, or 5.0 mg to 10.0 mg; e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg) and the C1D3 is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 3.0 mg to 50.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the second dosing cycle comprises a single dose (C2D1) of mosunetuzumab, wherein the C2D1 is equal to or greater than the C1D3 and is between 0.2 mg to 50 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 3.0 mg to 50.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.4 mg to 4.0 mg (e.g., 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.5 mg to 3.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 1.0 mg to 20.0 mg (e.g., 1.1 mg to 20.0 mg, 1.2 mg to 20.0 mg, 1.3 mg to 20.0 mg, 1.4 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 1.0 mg to 15.0 mg, 1.0 mg to 10.0 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 1.0 mg to 2.0 mg, 10.0 mg to 20.0 mg, 15.0 mg to 20.0 mg, 5.0 mg to 15.0 mg, or 5.0 mg to 10.0 mg; e.g., about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the C2D1 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.8 mg to 3.0 mg (e.g., 0.9 mg to 3.0 mg, 1.0 mg to 3.0 mg, 1.25 mg to 3.0 mg, 1.5 mg to 3.0 mg, 2.0 mg to 3.0 mg, 2.5 mg to 3.0 mg, 0.8 mg to 2.5 mg, 0.8 mg to 2.0 mg, 0.5 mg to 1.5 mg, 0.8 mg to 1.0 mg, 1.0 mg to 3.0 mg, 1.5 mg to 2.0 mg, or 0.9 mg to 1.1 mg; e.g., about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, or about 3.0 mg), the C1D2 is between 1.0 mg to 6.0 mg (e.g., 1.5 mg to 6.0 mg, 2.0 mg to 6.0 mg, 2.5 mg to 6.0 mg, 3.0 mg to 6.0 mg, 3.5 mg to 6.0 mg, 4.0 mg to 6.0 mg, 5.0 mg to 6.0 mg, 5.0 mg to 6.0 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 2.5 mg, 3.0 mg to 6.0 mg, 2.0 mg to 4.0 mg, 2.5 mg to 5.0 mg, or 2.5 mg to 3.5 mg; e.g., about 1.0 mg, about 1.1 mg, about 1.2 mg, about 1.3 mg, about 1.4 mg, about 1.5 mg, about 1.6 mg, about 1.7 mg, about 1.8 mg, 1.9 mg, about 2.0 mg, about 2.1 mg, about 2.2 mg, about 2.3 mg, about 2.4 mg, about 2.5 mg, about. 2.6 mg, about 2.7 mg, about 2.8 mg, about 2.9 mg, about 3.0 mg, about 3.5 mg, about 4.0 mg, about 4.5 mg, about 5.0 mg, about 5.5 mg, or about 6.0 mg), and the C1D3 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg); and (b) the C2D1 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg).

In some embodiments, the C1D1 and C1D2 are each less than the C1D3. In some embodiments, the C1D1 and C1D2 are about equal. In some embodiments, the C1D2 is greater than the C1D1 by about 50% to about 250% (e.g., the C1D2 is greater than the C1D1 by about 50% to about 225%, the C1D2 is greater than the C1D1 by about 50% to about 200%, the C1D2 is greater than the C1D1 by about 50% to about 175%, the C1D2 is greater than the C1D1 by about 50% to about 150%, the C1D2 is greater than the C1D1 by about 50% to about 125%, the C1D2 is greater than the C1D1 by about 50% to about 100%, or the C1D2 is greater than the C1D1 by about 50% to about 75%; e.g., the C1D2 is greater than the C1D1 by about 50%, the C1D2 is greater than the C1D1 by about 100%, the C1D2 is greater than the C1D1 by about 150%, the C1D2 is greater than the C1D1 by about 200%, or the C1D2 is greater than the C1D1 by about 250%).

In some embodiments, (a) the C1D1 is 0.8 mg, the C1D2 is 2.0 mg, the C1D3 is 4.2 mg, and the C2D1 is 4.2 mg; (b) the C1D1 is 1.0 mg, the C1D2 is 1.0 mg, the C1D3 is 3.0 mg, and the C2D1 is 30.0 mg; or (c) the C1D1 is 1.0 mg, the C1D2 is 2.0 mg, the C1D3 is 30.0 mg, and the C2D1 is 30.0 mg.

In some embodiments, the length of the first dosing cycle is 21 days (±1 day). In some embodiments, the method comprises administering to the subject the C1D1, the C1D2, and the C1D3 on or about Days 1, 8 (±1 day), and 15 (±1 day), respectively, of the first dosing cycle. In some embodiments, the length of the second dosing cycle is 28 days (±1 day). In some embodiments, the method comprises administering to the subject the C2D1 on Day 1 of the second dosing cycle.

In some embodiments, the dosing regimen comprises one or more additional dosing cycles (e.g., one dosing cycle, two dosing cycles, three dosing cycles, four dosing cycles, five dosing cycles, six dosing cycles, seven dosing cycles, eight dosing cycles, nine dosing cycles, ten dosing cycles, or more). In some embodiments, the dosing regimen comprises one to ten additional dosing cycles (e.g., one dosing cycle, two dosing cycles, three dosing cycles, four dosing cycles, five dosing cycles, six dosing cycles, seven dosing cycles, eight dosing cycles, nine dosing cycles, or ten dosing cycles). In some embodiments, the dosing regimen comprises ten additional dosing cycles. In some embodiments, the length of each of the one or more additional dosing cycles is 28 days (±1 day). In some embodiments, each of the one or more additional dosing cycles comprises an additional dose of mosunetuzumab. In some embodiments, the method comprises administering to the subject each additional dose of mosunetuzumab on Day 1 of each of the one or more additional dosing cycles. In some embodiments, each additional dose of mosunetuzumab is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg). In some embodiments, the dosing regimen comprises twelve dosing cycles comprising mosunetuzumab and eleven dosing cycles comprising lenalidomide.

In some embodiments, administering the effective amount of mosunetuzumab comprises administering mosunetuzumab according to a dosing regimen comprising twelve dosing cycles, wherein: (a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg (e.g., 0.05 mg to 4.0 mg, 0.1 mg to 4.0 mg, 0.2 mg to 4.0 mg, 0.3 mg to 4.0 mg, 0.4 mg to 4.0 mg, 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.1 mg to 3.5 mg, 0.1 mg to 3.0 mg, 0.1 mg to 2.5 mg, 0.1 mg to 2.0 mg, 0.1 mg to 1.5 mg, 0.1 mg to 1 mg, 0.1 mg to 0.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 0.05 mg to 20.0 mg (e.g., 0.1 mg to 20.0 mg, 0.2 mg to 20.0 mg, 0.3 mg to 20.0 mg, 0.4 mg to 20.0 mg, 0.5 mg to 20.0 mg, 1.0 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 0.5 mg to 15.0 mg, 0.5 mg to 10.0 mg, 0.5 mg to 9.0 mg, 0.5 mg to 8.0 mg, 0.5 mg to 7.0 mg, 0.5 mg to 6.0 mg, 0.5 mg to 5.0 mg, 0.5 mg to 4.0 mg, 0.5 mg to 3.0 mg, 0.5 mg to 2.0 mg, 0.5 mg to 1.0 mg, 1.0 mg to 15.0 mg, 1.0 mg to 10.0 mg, 1.0 mg to 8.0 mg, 1.0 mg to 6.0 mg, 1.0 mg to 4.0 mg, 1.0 mg to 3.0 mg, 10.0 mg to 20.0 mg, 15.0 mg to 20.0 mg, 5.0 mg to 15.0 mg, or 5.0 mg to 10.0 mg; e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the second to twelfth dosing cycles each comprises a single dose (C2D1-C12D1) of mosunetuzumab, wherein each single dose C2D1-C12D1 are equivalent in amount, is equal to or greater than the C1D3, and is between 0.2 mg to 50 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.4 mg to 4.0 mg (e.g., 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.5 mg to 3.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 1.0 mg to 20.0 mg (e.g., 1.1 mg to 20.0 mg, 1.2 mg to 20.0 mg, 1.3 mg to 20.0 mg, 1.4 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 1.0 mg to 15.0 mg, 1.0 mg to 10.0 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 1.0 mg to 2.0 mg, 10.0 mg to 20.0 mg, 15.0 mg to 20.0 mg, 5.0 mg to 15.0 mg, or 5.0 mg to 10.0 mg; e.g., about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) each single dose C2D1-C12D1 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.8 mg to 3.0 mg (e.g., 0.9 mg to 3.0 mg, 1.0 mg to 3.0 mg, 1.25 mg to 3.0 mg, 1.5 mg to 3.0 mg, 2.0 mg to 3.0 mg, 2.5 mg to 3.0 mg, 0.8 mg to 3.0 mg, 0.8 mg to 2.5 mg, 0.8 mg to 2.0 mg, 0.5 mg to 1.5 mg, 0.8 mg to 1.0 mg, 1.0 mg to 3.0 mg, 1.5 mg to 2.0 mg, or 0.9 mg to 1.1 mg; e.g., about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, or about 3.0 mg), the C1D2 is between 1.0 mg to 6.0 mg (e.g., 1.5 mg to 6.0 mg, 2.0 mg to 6.0 mg, 2.5 mg to 6.0 mg, 3.0 mg to 6.0 mg, 3.5 mg to 6.0 mg, 4.0 mg to 6.0 mg, 5.0 mg to 6.0 mg, 5.0 mg to 6.0 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 2.5 mg, 3.0 mg to 6.0 mg, 2.0 mg to 4.0 mg, 2.5 mg to 5.0 mg, or 2.5 mg to 3.5 mg; e.g., about 1.0 mg, about 1.1 mg, about 1.2 mg, about 1.3 mg, about 1.4 mg, about 1.5 mg, about 1.6 mg, about 1.7 mg, about 1.8 mg, 1.9 mg, about 2.0 mg, about 2.1 mg, about 2.2 mg, about 2.3 mg, about 2.4 mg, about 2.5 mg, about. 2.6 mg, about 2.7 mg, about 2.8 mg, about 2.9 mg, about 3.0 mg, about 3.5 mg, about 4.0 mg, about 4.5 mg, about 5.0 mg, about 5.5 mg, or about 6.0 mg), and the C1D3 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg); and (b) each single dose C2D1-C12D1 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg).

In some embodiments, the C1D1 and C1D2 are each less than the C1D3. In some embodiments, the C1D2 is greater than the C1D1 by about 50% to about 250% (e.g., the C1D2 is greater than the C1D1 by about 50% to about 225%, the C1D2 is greater than the C1D1 by about 50% to about 200%, the C1D2 is greater than the C1D1 by about 50% to about 175%, the C1D2 is greater than the C1D1 by about 50% to about 150%, the C1D2 is greater than the C1D1 by about 50% to about 125%, the C1D2 is greater than the C1D1 by about 50% to about 100%, or the C1D2 is greater than the C1D1 by about 50% to about 75%; e.g., the C1D2 is greater than the C1D1 by about 50%, the C1D2 is greater than the C1D1 by about 100%, the C1D2 is greater than the C1D1 by about 150%, the C1D2 is greater than the C1D1 by about 200%, or the C1D2 is greater than the C1D1 by about 250%).

In some embodiments, (a) the C1D1 is 0.8 mg, the C1D2 is 2.0 mg, the C1D3 is 4.2 mg, and each single dose C2D1-C12D1 is 4.2 mg; (b) the C1D1 is 1.0 mg, the C1D2 is 1.0 mg, the C1D3 is 3.0 mg, and each single dose C2D1-C12D1 is 30.0 mg; or (c) the C1D1 is 1.0 mg, the C1D2 is 2.0 mg, the C1D3 is 30.0 mg, and each single dose C2D1-C12D1 is 30.0 mg.

In some embodiments, the length of the first dosing cycle is 21 days (±1 day). In some embodiments, the method comprises administering to the subject the C1D1, the C1D2, and the C1D3 on or about Days 1, 8 (±1 day), and 15 (±1 day), respectively, of the first dosing cycle. In some embodiments, the length of each of the second to twelfth dosing cycles is 28 days (±1 day). In some embodiments, the method comprises administering to the subject each of the C2D1-C12D1 on Day 1 of each respective dosing cycle. In some embodiments, the length of each of the second to twelfth dosing cycles is 28 days (±1 day).

In some embodiments, mosunetuzumab is administered intravenously.

In some embodiments, lenalidomide is administered during the second and subsequent cycles. In some embodiments, lenalidomide is not administered during the first cycle. In some embodiments, lenalidomide is administered daily. In some embodiments, lenalidomide is administered daily on the first 21 days of each dosing cycle comprising administration of lenalidomide. In some embodiments, lenalidomide is not administered on the last 7 days of each dosing cycle comprising administration of lenalidomide. In some embodiments, lenalidomide is administered at a dose of 20 mg. In some embodiments, lenalidomide is administered orally.

In some embodiments, the subject was previously treated with at least one anti-CD20 monoclonal antibody. In some embodiments, the subject is relapsed or refractory to the treatment comprising the anti-CD20 monoclonal antibody. In some embodiments, the anti-CD20 monoclonal antibody is obinutuzumab or rituximab.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included obinutuzumab; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-21 of the second dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included obinutuzumab; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprise a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included rituximab; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab administered on Day 1 of the second dosing cycle, wherein the C2D1 is 30 mg, and (c) the second dosing cycle further comprises administering 20 mg lenalidomide daily orally on Days 1-21 of the second dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included rituximab; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprises a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle.

In some embodiments, the subject has received only one prior line of systemic therapy and either: (a) has a Follicular Lymphoma International Prognostic Index (FLIPI; Solal-Céligny et al. Blood. 2004; 104 (5): 1258-1265.) score of 2-5 (e.g., a score of 2, 3, 4, or 5), (b) has been refractory to prior obinutuzumab treatment, (c) has been refractory to prior rituximab treatment, or (d) has disease progression within 24 months after initiation of prior therapy. In some embodiments, the subject has not been treated with obinutuzumab or rituximab for at least 4 weeks (e.g., 4 weeks, 6 weeks, 8 weeks, 10 weeks, 12 weeks, 24 weeks, 36 weeks, 48 weeks, 60 weeks, or more) prior to being administered the effective amount of mosunetuzumab and lenalidomide.

In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments, the synergistic effect is a complete response, as defined by Lugano 2014 criteria (Cheson B D, et al. J Clin Oncol 2014; 32:1-9).

In some embodiments, the FL is histologically documented as Grade 1, 2, or 3a, but not 3b according to the World Health Organization classification of lymphoid neoplasms (as referenced in Swerdlow S H, et al. Blood 2016; 127:2375-90).

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-21 of the second dosing cycle. In some embodiments, at least one chemo-immunotherapy regimen included an anti-CD20 monoclonal antibody (e.g., rituximab or obinutuzumab).

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprises a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle. In some embodiments, at least one chemo-immunotherapy regimen included an anti-CD20 monoclonal antibody (e.g., rituximab or obinutuzumab).

In some embodiments, each subject has received only one prior line of systemic therapy and either: (a) has a Follicular Lymphoma International Prognostic Index (FLIPI; Solal-Céligny et al. Blood. 2004; 104 (5): 1258-1265.) score of 2-5 (e.g., a score of 2, 3, 4, or 5), (b) has been refractory to prior anti-CD20 monoclonal antibody treatment, or (c) has disease progression within 24 months after initiation of prior therapy.

In some embodiments, the FL of each subject is histologically documented as Grade 1, 2, or 3a, but not 3b according to the World Health Organization classification of lymphoid neoplasms (as referenced in Swerdlow S H, et al. Blood 2016; 127:2375-90).

In some embodiments, each subject has not been treated with the anti-CD20 monoclonal antibody for at least 4 weeks (e.g., 4 weeks, 6 weeks, 8 weeks, 10 weeks, 12 weeks, 24 weeks, 36 weeks, 48 weeks, 60 weeks, or more) prior to being administered the effective amount of mosunetuzumab and lenalidomide.

In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments, the synergistic effect is a complete response, as defined by Lugano 2014 criteria (Cheson B D, et al. J Clin Oncol 2014; 32:1-9).

In some embodiments, the incidence of an adverse event is not significantly higher than if mosunetuzumab was administered alone to the population of subjects.

In some embodiments, the incidence of an adverse event is not significantly higher than if lenalidomide is not administered to the population of subjects.

In some embodiments, the incidence rate of cytokine release syndrome as defined by the American Society of Transplantation and Cellular Therapy (ASTCT) Consensus Grading for Cytokine-Release Syndrome (“ASTCT CRS grading;” Lee et al., Biol Blood Marrow Transplant 2019) is less than 45% (e.g., less than 40%, less than 35%, less than 30%, less than 25%, less than 20%, less than 15%, less than 10%, less than 5%, less than 3%, or less than 1%; e.g., between about 0% to about 50%, between about 5% to about 40%, between about 5% to about 20%, between about 5% to about 10%, between about 20% to about 45%, between about 30% to about 40%, between about 20% to about 40%, between about 15% to about 35%, between about 15% to about 25%, between about 35% to about 45%, or between about 25% to about 45%; e.g., about 45%, about 40%, about 35%, about 30%, about 25%, about 20%, about 15%, about 10%, about 5%, about 1%, or about 0%). In some embodiments, the incidence rate of cytokine release syndrome as defined by the American Society of Transplantation and Cellular Therapy (ASTCT) Consensus Grading for Cytokine-Release Syndrome (“ASTCT CRS grading”) is less than 35%. In some embodiments, the incidence rate of cytokine release syndrome as defined by the American Society of Transplantation and Cellular Therapy (ASTCT) Consensus Grading for Cytokine-Release Syndrome (“ASTCT CRS grading”) is less than 25%.

In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 10% (e.g., less than 9%, less than 8%, less than 7%, less than 6%, less than 5%, less than 4%, less than 3%, less than 2%, less than 1%; e.g., between 0% and 10%, between 0% and 9%, between 0% and 8%, between 0% and 7%, between 0% and 6%, between 0% and 5%, between 0% and 4%, between 0% and 3%, between 0% and 2%, between 0% and 1%, between 1% and 3%, between 1% and 5%, between 1% and 10%, between 3% and 5%, between 5% and 8%, between 5% and 10%, between 8% and 10%. In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 5%. In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 3%. In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 1%.

In some embodiments, the incidence rate of neutropenia is less than 40% (e.g., less than 35%, less than 30%, less than 25%, less than 20%, less than 15%, less than 10%, less than 5%, less than 3%, or less than 1%; e.g., between about 0% to about 40%, between about 5% to about 40%, between about 5% to about 20%, between about 5% to about 10%, between about 20% to about 40%, between about 30% to about 40%, between about 15% to about 35%, between about 15% to about 25%, between about 35% to about 40%, or between about 25% to about 40%; e.g., about 40%, about 35%, about 30%, about 25%, about 20%, about 15%, about 10%, about 5%, about 3%, about 1%, or about 0%). In some embodiments, the incidence rate of neutropenia is less than 30%. In some embodiments, the incidence rate of neutropenia is less than 20%.

In some embodiments, the overall response rate is at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more; e.g., between 80%-100%, between 85-100%, between 87-100%, between 90-100%, between 95-100%, between 80%-90%, between 80-85%, between 85-97%, between 85-95%, between 85-90%, between 85-87%, between 90-95%, or between 93-97%; e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more). In some embodiments, the overall response rate is at least 90%. In some embodiments, the overall response rate is at least 95%. In some embodiments, the overall response rate is at least 99%.

In some embodiments, the complete response rate is at least 65% (e.g., at least 70%, at least 75%, at least 80%, at least 85%, at least 90%, at least 95%, or more; e.g., between 65-100%, between 75-100%, between 85-100%, between 95-100%, between 65-90%, between 65-80%, between 65-70%, between 65-75%, or between 75-85%; e.g., about 65%, about 66%, about 67%, about 68%, about 69%, about 70%, about 71%, about 72%, about 73%, about 74%, about 75%, about 76%, about 77%, about 80%, about 85%, about 90%, or more). In some embodiments, the complete response rate is at least 75%. In some embodiments, the complete response rate is at least 85%.

In some embodiments, each subject was previously treated with at least one anti-CD20 monoclonal antibody. In some embodiments, the anti-CD20 monoclonal antibody is obinutuzumab or rituximab.

In some embodiments, the subject is human. In some embodiments, each subject in the population is human.

In some embodiments, the subject exhibits a reduction in tumor burden after being administered an effective amount of mosunetuzumab and an effective amount of lenalidomide. In some embodiments, the reduction in tumor burden is determined by computed tomography (CT). In some embodiments, the reduction in tumor burden is a decrease in the sum of the product of the diameters (SPD) of target lesions. In some embodiments, the decrease in SPD is at least 40% (e.g., at least 50%, at least 60%, at least 70%, at least 80%, at least 90%, at least 95%, at least 96%, at least 97%, at least 98%, at least 99%, or more; e.g., between 40-100%, between 40-80%, between 40-60%, between 50-70%, between 70-90%, between 75-85%, between 60-100%, between 55-65%, between 80-100%; e.g., about 40%, about 45%, about 50%, about 55%, about 60%, about 65%, about 70%, about 75%, about 80%, about 85%, about 90%, about 95%, or about 100%). In some embodiments, the decrease in SPD is at least 60%. In some embodiments, the decrease in SPD is at least 80%.

In some embodiments, at least 45% (e.g., at least 50%, at least 60%, at least 70%, at least 80%, at least 90%, at least 95%, at least 96%, at least 97%, at least 98%, at least 99%, or more; e.g., between 45-100%, between 45-80%, between 45-60%, between 50-70%, between 55-65%, between 70-90%, between 70-80%, between 60-100%, between 80-100%; e.g., about 45%, about 50%, about 55%, about 60%, about 65%, about 70%, about 75%, about 80%, about 85%, about 90%, about 95%, or about 100%) of subjects in the population exhibit a reduction in tumor burden after being administered an effective amount of mosunetuzumab and an effective amount of lenalidomide. In some embodiments, at least 60% of subjects in the population exhibit a reduction in tumor burden after being administered an effective amount of mosunetuzumab and an effective amount of lenalidomide. In some embodiments, at least 75% of subjects in the population exhibit a reduction in tumor burden after being administered an effective amount of mosunetuzumab and an effective amount of lenalidomide. In some embodiments, the reduction in tumor burden is determined by computed tomography (CT).

In some embodiments, the reduction in tumor burden is a decrease in the sum of the product of the diameters (SPD) of target lesions. In some embodiments, the decrease in SPD is at least 40% (e.g., at least 50%, at least 60%, at least 70%, at least 80%, at least 90%, at least 95%, at least 96%, at least 97%, at least 98%, at least 99%, or more; e.g., between 40-100%, between 40-80%, between 40-60%, between 50-70%, between 70-90%, between 75-85%, between 60-100%, between 55-65%, between 80-100%; e.g., about 40%, about 45%, about 50%, about 55%, about 60%, about 65%, about 70%, about 75%, about 80%, about 85%, about 90%, about 95%, or about 100%). In some embodiments, the decrease in SPD is at least 60%. In some embodiments, the decrease in SPD is at least 80%.

In some embodiments, the dosing regimen further comprises administration of a corticosteroid. In some embodiments, the corticosteroid is administered to the subject during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subject on Days 1, 8 (±1 day), and 15 (±1 day), respectively, of the first dosing cycle. In some embodiments, wherein each single dose C1D1-C1D3 of the corticosteroid is administered to the subject before administration of the C1D1-C1D3, respectively, of mosunetuzumab. In some embodiments, each single dose C1D1-C1D3 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab.

In some embodiments, the corticosteroid is administered to the subject on the first dosing cycle and not on the second dosing cycle.

In some embodiments, the corticosteroid is administered to the subject on the second dosing cycle. In some embodiments, the second dosing cycle comprises a single dose (C2D1) of the corticosteroid. In some embodiments, the C2D1 of the corticosteroid is administered to the subject on Day 1 of the second dosing cycle. In some embodiments, the C2D1 of the corticosteroid is administered to the subject before administration of the C2D1 of mosunetuzumab. In some embodiments, the C2D1 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, the dosing regimen further comprises administration of a corticosteroid. In some embodiments, the corticosteroid is administered to the subjects during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subjects on Days 1, 8 (±1 day), and 15 (±1 day), respectively, of the first dosing cycle. In some embodiments, each single dose C1D1-C1D3 of the corticosteroid is administered to the subjects before administration of the C1D1-C1D3, respectively, of mosunetuzumab. In some embodiments, each single dose C1D1-C1D3 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab.

In some embodiments, the corticosteroid is administered to the subjects on the first dosing cycle and not on the second dosing cycle.

In some embodiments, the corticosteroid is administered to the subjects on the second dosing cycle. In some embodiments, the second dosing cycle comprises a single dose (C2D1) of the corticosteroid. In some embodiments, the C2D1 of the corticosteroid is administered to the subjects on Day 1 of the second dosing cycle. In some embodiments, the C2D1 of the corticosteroid is administered to the subjects before administration of the C2D1 of mosunetuzumab. In some embodiments, the C2D1 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, each additional dosing cycle comprises administering to the subject an additional dose of the corticosteroid. In some embodiments, each additional dose of the corticosteroid is administered on Day 1 of each additional dosing cycle. In some embodiments, each additional dose of the corticosteroid is administered to the subject before administration of each additional dose of mosunetuzumab. In some embodiments, each additional dose of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of each additional dose of mosunetuzumab.

In some embodiments, the corticosteroid is administered intravenously. In some embodiments, the corticosteroid is dexamethasone. In some embodiments, each dose of dexamethasone is about 10 mg (e.g., about 1, 2, 3, 4, 5, 6, 7, 8, 9, 10, 11, 12, 13, 14, 15, 16, 17, 18, 19, or 20 mg).

### BRIEF DESCRIPTION OF THE DRAWINGS

FIG. 1A is a schematic of the study design described in Example 1 for the dosing regimen of the combination therapy of mosunetuzumab and lenalidomide. C=cycle; D=day; FL=follicular lymphoma; IMC=Internal Monitoring Committee; IV=intravenous; Len=lenalidomide; Mosun=mosunetuzumab; RP2D=recommended Phase II dose; R/R=relapsed or refractory. Note: Cycle 1 is 21 days; Cycle 2 and onward is 28 days.

FIG. 1B is a schematic of the dosing regimen described in Example 1. Lenalidomide begins at cycle 2 to minimize risk with mosunetuzumab initiation during Cycle 1, when CRS risk is highest.

FIG. 2 is a butterfly plot showing adverse events that occurred in ≥10% of safety-evaluable patients described in Example 1 classified by MedDRA System Organ Class and MedDRA Preferred Term. Based on MedDRA version 23.1. Grade of adverse events is shown in legend. Left side shows all adverse events. Right side shows adverse events attributable to treatment with mosunetuzumab (Mosun) or lenalidomide. Clinical cut-off date (CCOD) of Apr. 5, 2021.

FIG. 3 is a butterfly plot showing adverse events that occurred in ≥10% of safety-evaluable patients described in Example 1 classified by MedDRA System Organ Class and MedDRA Preferred Term. Based on MedDRA version 23.1. Grade of adverse events is shown in legend. Left side shows all adverse events. Right side shows adverse events attributable to treatment with mosunetuzumab (Mosun). CCOD of Apr. 5, 2021.

FIG. 4 is a butterfly plot showing adverse events that occurred in ≥10% of safety-evaluable patients described in Example 1 classified by MedDRA System Organ Class and MedDRA Preferred Term. Based on MedDRA version 23.1. Grade of adverse events is shown in legend. Left side shows all adverse events. Right side shows adverse events attributable to treatment with lenalidomide. CCOD of Apr. 5, 2021.

FIG. 5 is a swimlane plot showing onset of cytokine release syndrome (CRS) events in 5 patients treated with the 1/2/30 mg dosing regimen of mosunetuzumab described in Example 1. Onset of Grade 1 or Grade 2 CRS is indicated by the light or dark diamond, respectively. CCOD of Apr. 5, 2021.

FIG. 6 is a plot showing observed concentration-time data of mosunetuzumab (in combination with lenalidomide) from Study CO41942 overlaid with popPK model simulations based on mosunetuzumab monotherapy data from Study GO29781; Simulations were performed at 1/2/30 mg Q4W. Filled red circles and lines represent the individual and mean observed mosunetuzumab concentration measurements from Study CO41942 at 1/2/30 mg IV dose in combination with lenalidomide. Shaded area represents simulated concentration-time profile for mosunetuzumab monotherapy based on the preliminary population PK model simulations (5^{th}-95^{th} prediction interval) which was developed based on clinical data from Study GO29781.

FIG. 7 is a butterfly plot showing adverse events that occurred in ≥10% of safety-evaluable patients described in Example 3 classified by MedDRA System Organ Class and MedDRA Preferred Term. Based on MedDRA version 23.1. Grade of adverse events is shown in legend. Left side shows all adverse events. Right side shows adverse events attributable to treatment with mosunetuzumab (Mosun) or lenalidomide. CCOD of Sep. 13, 2021.

FIG. 8 is a bar graph showing cytokine release syndrome (CRS) events by dosing cycle (C) and day (D) and by CRS Grade (Gr). Values below the graph show the dose of mosunetuzumab associated with each dosing cycle (C) and day (D). The C1D1 dose is administered on Cycle 1 Day 1, the C1D2 dose is administered on Cycle 1 Day 8, and the C1D3 dose is administered on Cycle 1 Day 15. Subsequent doses are administered on Day 1 of each respective dosing cycle (C2, C3+).

FIG. 9 is a bar graph showing neutropenia and febrile neutropenia events by dosing cycle (C) and by neutropenia Grade (Gr). The number of patients who received granulocyte colony-stimulating factor (G-CSF) per dosing cycle (C) is shown below the graph.

FIG. 10 is a bar graph showing anti-tumor activity in patients with 1 diagnostic computed tomography (CT) scan at response assessment. The number above each bar shows % change from baseline. Changes in tumor burden are quantified by comparing the sum of the product of the diameters (SPD) of target lesions to baseline measurements.

FIG. 11 is a bar graph showing the overall response rate (ORR), complete response rate (CR), and partial response rate (PR) in the 29 patients treated with the 1/2/30 mg dosing regimen of mosunetuzumab and 20 mg oral lenalidomide described in Example 3. Response rates are shown for 29 patients as of data cut-off date of Sep. 13, 2021. 95% confidence interval (CI) for each response rate value is shown in parentheses below. Response rates are determined by positron emission tomography-computed tomography (PET-CT).

FIG. 12 is a swimlane plot showing duration of response in all 29 patients treated with the 1/2/30 mg dosing regimen of mosunetuzumab and 20 mg oral lenalidomide described in Example 3. Duration of response are shown for 29 patients as of data cut-off date of Sep. 13, 2021.

### DETAILED DESCRIPTION

The present invention relates to methods of treating a subject having a relapsed and/or refractory (R/R) follicular lymphoma (FL) by administration of mosunetuzumab and lenalidomide as a combination therapy. In particular, the present invention relates to methods of treating a subject having a R/R FL that is R/R to at least one prior systemic therapy (e.g., one prior treatment with a chemo-immunotherapy regimen) which included an anti-CD20 antibody (e.g., an anti-CD20 monoclonal antibody, e.g., rituximab or obinutuzumab) or other treatment. The method comprises administering the effective amount of mosunetuzumab according to a dosing regimen comprising at least a first dosing cycle and a second dosing cycle, wherein: (a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg and the C1D3 is between 0.2 mg to 50.0 mg; and (b) the second dosing cycle comprises a single dose (C2D1) of mosunetuzumab, wherein the C2D1 is equal to or greater than the C1D3 and is between 0.2 mg to 50 mg.

The invention is based, in part, on the discovery that combination therapy involving intravenous administration of mosunetuzumab and oral administration of lenalidomide over multiple dosing cycles (e.g., wherein the first dosing cycle is a step-up, fractionated dosing cycle) exhibits synergistic effects between mosunetuzumab and lenalidomide and can effectively treat subjects having relapsed/refractory (R/R) follicular lymphoma (FL), in particular those who are R/R to prior systemic therapy including an anti-CD20 antibody (e.g., an anti-CD20 monoclonal antibody, e.g., rituximab or obinutuzumab; e.g., R-CHOP) while maintaining an acceptable safety profile (e.g., with respect to frequency and severity of adverse events).

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N. Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

The terms “cancer” and “cancerous” refer to or describe the physiological condition in mammals that is typically characterized by unregulated cell growth. Examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be relapsed or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, μ heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a relapsed and/or refractory FL or transformed FL), diffuse large B cell lymphoma (DLBCL) (e.g., a relapsed or refractory DLBCL or a Richter's transformation), MCL, high grade B-cell lymphoma, or PMLBCL). In another embodiment, the B cell proliferative disorder is a leukemia, such as chronic lymphocytic leukemia (CLL). In one embodiment, the B-cell proliferative disorder is a relapsed and/or refractory FL.

By “Follicular Lymphoma International Prognostic Index” or “FLIPI” is meant a scoring system or index for determining prognostic risk of patients (e.g., with cancer; e.g., an NHL; e.g., a follicular lymphoma (FL)). FLIPI score ranges from 0-5, depending on how many of the five conditions or risk factors a patient may have among the following: (i) age≥60 years; (ii) Ann Arbor stage III-IV; hemoglobin level≤120 g/L; serum lactate dehydrogenase (LDH) level≥upper limit of normal (ULN) (e.g., >280 units/L); and (v) number of nodal sites >4. See e.g., Solal-Céligny et al. Blood. 2004; 104 (5): 1258-1265. at Table 4.

As used herein, the terms “Ann Arbor staging” or “Ann Arbor stages” refers to a system for classification of stages of lymphoma (e.g., non-Hodgkin's lymphoma (NHL); e.g., a DLBCL, an FL, an MCL, a high-grade B cell lymphoma, a PMLBCL, or a CLL). Lymphomas (e.g., NHLs) can be classified as one of four Ann Arbor stages. Stage I refers to lymphomas exhibiting involvement of a single lymph node region or of a single extralymphatic organ or site. Stage II refers to lymphomas exhibiting involvement of 2 or more lymph node regions on the same side of the diaphragm. Stage III refers to lymphomas exhibiting involvement of lymph node regions on both sides of the diaphragm (III), which may also be accompanied by localized involvement of extralymphatic organ or site or by involvement of the spleen, or both. Stage IV refers to lymphomas exhibiting diffuse or disseminated involvement of 1 or more extralymphatic organs or tissues with or without associated lymph node enlargement. Liver involvement is always considered to be diffuse, and, thus, always considered Ann Arbor stage IV. Lymphatic structures include the lymph nodes, thymus, spleen, appendix, Waldeyer's ring, and Peyer's patches. See Carbone, P. P. et al., Cancer Res. 1971, 31(11):1860-1861.

“Refractory disease” is defined as no complete remission to at least a first-line therapy. In one embodiment, refractory disease defined as no response to or relapse within 6 months of prior therapy. In one embodiment, refractory disease is characterized by one or more of the following: progressive disease (PD) as best response to first-line therapy, stable disease (SD) as best response after at least one first line therapy (e.g., at least one containing an anti CD20-directed therapy, e.g., including an anti-CD20 antibody, e.g., an anti-CD20 monoclonal antibody, e.g., rituximab or obinutuzumab), or partial response (PR) as best response, and biopsy-proven residual disease or disease progression after the partial response. “Relapsed disease” is defined as complete remission to first-line therapy. In one embodiment, disease relapse is proven by biopsy. In one embodiment, patients have relapsed after or failed to respond to at least one prior systemic treatment regimen (e.g., at least one containing an anti CD20-directed therapy, e.g., including an anti-CD20 antibody, e.g., an anti-CD20 monoclonal antibody, e.g., rituximab or obinutuzumab).

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a relapsed and/or refractory FL). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with mosunetuzumab using the step-up dosing regimen of the invention relative to unchanging, preset dosing with the target dose of mosunetuzumab. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the R/R FL being treated, the presence or size of metastases, or the size of the primary tumor. In yet other embodiments, reducing or inhibiting cancer relapse means to reduce or inhibit tumor or cancer relapse, or tumor or cancer progression.

As used herein, “administering” is meant a method of giving a dosage of a compound (e.g., a bispecific antibody, e.g., an anti-CD20/anti-CD3 bispecific antibody, e.g., mosunetuzumab) or a composition (e.g., a pharmaceutical composition, e.g., a pharmaceutical composition including a bispecific antibody (e.g., mosunetuzumab)) to a subject. The compounds and/or compositions utilized in the methods described herein can be administered intravenously (e.g., by intravenous infusion).

A “fixed” or “flat” dose of a therapeutic agent (e.g., a bispecific antibody) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

A “subject” or an “individual” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In certain embodiments, the subject or individual is a human.

“Individual response” or “response” can be assessed using any endpoint indicating a benefit to the subject, including, without limitation, (1) inhibition, to some extent, of disease progression (e.g., progression of a R/R FL, including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the R/R FL; (6) increase or extend in the length of survival, including overall survival and progression-free survival; and/or (9) decreased mortality at a given point of time following treatment.

As used herein, “complete response” or “CR” refers to disappearance of all target lesions (i.e., all evidence of disease).

As used herein, “partial response” or “PR” refers to at least a 30% decrease in the sum of the longest diameters (SLD) of target lesions, taking as reference the baseline SLD, or at least a 50% decrease in the sum of the product of the diameters (SPD) of target lesions, taking as reference the baseline SPD.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, or death from any cause within 30 days of the last dose of a treatment, whichever occurs first.

As used herein, “tumor burden” refers to the total amount of tumor (e.g., tumor cells or tumor mass) in a subject (e.g., a human subject) having a cancer, e.g., an NHL, e.g., an FL. In some embodiments, tumor burden is defined as the sum of diameters of target lesions or the sum of the product of target lesions. In a particular embodiment, tumor burden is defined as the sum of the product of the diameters of (SPD) target lesions. In some embodiments, the diameters of target lesions is quantified by computed tomography (CT).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., a R/R FL) does not get worse. Progression-free survival may include the amount of time patients have experienced a complete response or a partial response, as well as the amount of time patients have experienced stable disease.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, “delaying progression” of a disorder or disease means to defer, hinder, slow, retard, stabilize, and/or postpone development of the disease or disorder (e.g., a relapsed and/or refractory (R/R) follicular lymphoma (FL)). This delay can be of varying lengths of time, depending on the history of the disease and/or subject being treated. As is evident to one skilled in the art, a sufficient or significant delay can, in effect, encompass prevention, in that the subject does not develop the disease. For example, in a late-stage cancer, development of central nervous system (CNS) metastasis, may be delayed.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full-length antibody,” “intact antibody,” and “whole antibody” are used herein interchangeably to refer to an antibody having a structure substantially similar to a native antibody structure or having heavy chains that contain an Fc region as defined herein.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′₂, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat. Acad. Sci. USA, 103:3557-3562 (2006) regarding human antibodies generated via a human B-cell hybridoma technology.

The term “variable region” or “variable domain” refers to the domain of an antibody heavy or light chain that is involved in binding the antibody (i.e., mosunetuzumab) to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody (i.e., mosunetuzumab) will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody (i.e., mosunetuzumab) as being obtained from a substantially homogeneous population of antibodies, and is not to be construed as requiring production of the antibody by any particular method. For example, the monoclonal antibodies (i.e., mosunetuzumab) to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species. In some embodiments, the anti-CD20 antibody is a monoclonal antibody. In some embodiments, the anti-CD20 antibody or anti-CD20 monoclonal antibody is rituximab. In some embodiments, the anti-CD20 antibody or anti-CD20 monoclonal antibody is obinutuzumab.

As used herein, the term “rituximab” or “RITUXAN®” refers to an anti-CD20 antibody (e.g., anti-CD20 monoclonal antibody) having the Proposed International Nonproprietary Names for Pharmaceutical Substances (Proposed INN) List 77 (WHO Drug Information, Vol. 11, No. 2, 1997, p. 99), or the CAS Registry Number 174722-31-7.

As used herein, the term “obinutuzumab” or “GAZYVA®” refers to an anti-CD20 antibody (e.g., anti-CD20 monoclonal antibody) having the Proposed International Nonproprietary Names for Pharmaceutical Substances (Proposed INN) List 99 (WHO Drug Information, Vol. 22, No. 2, 2008, p. 396), Proposed International Nonproprietary Names for Pharmaceutical Substances (Proposed INN) List 108 (WHO Drug Information, Vol. 26, No. 4, 2012, p. 453), or the CAS Registry Number 949142-50-1.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

The terms “anti-CD20/anti-CD3 bispecific antibody,” “bispecific anti-CD20/anti-CD3 antibody,” and “antibody that binds to CD20 and CD3,” or variants thereof, refer to a multispecific antibody (e.g., a bispecific antibody) that is capable of binding to CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, a bispecific antibody that binds to CD20 and CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, a bispecific antibody that binds to CD20 and CD3 binds to an epitope of CD3 that is conserved among CD3 from different species and/or an epitope of CD20 that is conserved among CD20 from different species. In one embodiment, the bispecific antibody binds monovalently to CD20 and binds monovalently to CD3. In some embodiments, a bispecific antibody that binds to CD20 and CD3 is mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 303), or the CAS Registry Number 1905409-39-3.

As used herein, the term “lenalidomide” refers to a compound having the CAS Registry Number 191732-72-6 and IUPAC name (3RS)-3-(4-Amino-1-oxo-1,3-dihydro-2H-isoindol-2-yl)piperidine-2,6-dione. Lenalidomide is also known by tradenames including REVLIMID®, linamid, and lenalid. Lenalidomide has the DrugBank Accession Number DB00480, PubChem CID 216326, and chemical formula C₁₃H₁₃N₃O₃.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN® (DNASTAR®) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX® operating system, including digital UNIX® V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “chemotherapeutic agent” refers to a compound useful in the treatment of a cancer, such as a R/R FL. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, dacarbazine, mechlorethamine, cisplatin, methotrexate, 5-fluorouracil, and ara-C); and (x) pharmaceutically acceptable salts, acids, prodrugs, and derivatives of any of the above.

The term “chemo-immunotherapy” refers to combination therapy that includes both chemotherapy drugs and immunotherapeutic agents. In some embodiments, chemo-immunotherapy is used to treat a cancer, e.g., a CD20-positive cancer, e.g., a NHL, e.g., a FL. In some embodiments, immunotherapeutic agents include an antibody, e.g., an anti-CD20 antibody (e.g., an anti-CD20 monoclonal antibody). In some embodiments, the anti-CD20 antibody or anti-CD20 monoclonal antibody is rituximab or obinutuzumab. In some embodiments, chemo-immunotherapy includes R-CHOP.

The term “R-CHOP” as used herein refers to a treatment comprising rituximab plus cyclophosphamide, doxorubicin, vincristine, and prednisone. In some embodiments, R-CHOP is a chemotherapy treatment or regimen used in the treatment of a cancer, optionally a B cell proliferative disorder (e.g., a non-Hodgkin's lymphoma; e.g., a DLBCL or a high grade B-cell lymphoma). In some embodiments, R-CHOP is the standard of care (SOC) or standard therapy to be administered to a subject to treat the cancer, optionally the B cell proliferative disorder (e.g., the non-Hodgkin's lymphoma; e.g., the DLBCL or the high grade B-cell lymphoma). In some embodiments, R-CHOP is the standard front-line or first-line therapy to be administered to a previously untreated subject. In some embodiments, R-CHOP is administered every 3 weeks (in 21-day dosing cycles) for 3 to 6 dosing cycles. In some embodiments, the dosing regimen for R-CHOP therapy comprises 21-day dosing cycles, wherein during each dosing cycle, the subject is administered 375 mg/m² rituximab IV, and also administered cyclophosphamide, doxorubicin, vincristine, and prednisone. In some embodiments, the dosing regimen for R-CHOP therapy further comprises 750 mg/m² cyclophosphamide IV, 50 mg/m² doxorubicin IV, 1.4 mg/m² vincristine IV, and 5 days of 100 mg or 40 mg/m² per day prednisone oral. In some embodiments, a maximum single dose of vincristine is 2 mg.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., ²¹¹At, ¹³¹I, ¹²⁵I, ⁹⁰I, ¹⁸⁶Re, ¹⁸⁸Re, ¹⁵³Sm, ²¹²Bi, ³²P, ²¹²Pb, and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “package insert” is used to refer to instructions customarily included in commercial packages of therapeutic products, that contain information about the indications, usage, dosage, administration, combination therapy, contraindications and/or warnings concerning the use of such therapeutic products.

The term “synergistic effect” is used to refer to an effect resulting from the combination use of two or more therapeutic products (e.g., mosunetuzumab and lenalidomide), which is greater than the sum of the effects observed when the two or more therapeutic products are used individually. In some embodiments, synergistic effect is observed in the response rate (e.g., complete response rate or overall response rate) of a population of subjects having relapsed and/or refractory (R/R) non-Hodgkin's Lymphoma (NHL; e.g., R/R follicular lymphoma (FL)) treated with a combination therapy comprising mosunetuzumab and lenalidomide. In some embodiments, a population of subjects having R/R FL exhibits a higher complete response rate when treated with combination therapy comprising mosunetuzumab and lenalidomide compared to a population of subjects treated with monotherapy mosunetuzumab or lenalidomide. In some embodiments, a population of subjects having R/R FL exhibits a higher overall response rate when treated with combination therapy comprising mosunetuzumab and lenalidomide compared to a population of subjects treated with monotherapy mosunetuzumab or lenalidomide.

### III. Therapeutic Methods

Provided herein are methods of treating a subject having a relapsed and/or refractory (R/R) follicular lymphoma (FL) by administration of mosunetuzumab and lenalidomide as a combination therapy. In particular, the present invention relates to methods of treating a subject having a R/R FL that is R/R to at least one prior systemic therapy (e.g., one prior treatment with a cherno-immunotherapy regimen) which included an anti-CD20 antibody (e.g., an anti-CD20 monoclonal antibody; e.g., rituximab or obinutuzumab) or other treatment. In some embodiments, the FL is a Graded FL (e.g., Grade 1, 2, or 3a, but not Grade 3b FL). In some embodiments, the FL of each subject is histologically documented as Grade 1, 2, or 3a, but not 3b according to the World Health Organization classification of lymphoid neoplasms (as referenced in Swerdlow S H, et al. Blood 2016; 127:2375-90). In some embodiments, the subject has received only one prior line of systemic therapy and either: (a) has a Follicular Lymphoma International Prognostic Index (FLIPI; Solal-Céligny et al. Blood. 2004; 104 (5): 1258-1265.) score of 2-5 (e.g., a score of 2, 3, 4, or 5), (b) has been refractory to prior anti-CD20 monoclonal antibody treatment, or (c) has disease progression within 24 months after initiation of prior therapy. In some embodiments, each subject has not been treated with the anti-CD20 monoclonal antibody for at least 4 weeks (e.g., 4 weeks, 6 weeks, 8 weeks, 10 weeks, 12 weeks, 24 weeks, 36 weeks, 48 weeks, 60 weeks, or more) prior to being administered the effective amount of mosunetuzumab and lenalidomide. In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments the synergistic effect is a complete response, as defined by Lugano 2014 criteria (Cheson B D, et al. J Clin Oncol 2014; 32:1-9).

### A. Therapeutic Methods for Dosing of Mosunetuzumab

The present invention relates to methods of treating a subject having a relapsed and/or refractory (R/R) follicular lymphoma (FL) by administration of mosunetuzumab and lenalidomide as a combination therapy. In particular, the present invention relates to methods of treating a subject having a R/R FL that is R/R to at least one prior systemic therapy (e.g., one prior treatment with a chemo-immunotherapy regimen) which included an anti-CD20 antibody (e.g., an anti-CD20 monoclonal antibody; e.g., rituximab or obinutuzumab) or other treatment. In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments the synergistic effect is a complete response, as defined by Lugano 2014 criteria (Cheson B D, et al. J Clin Oncol 2014; 32:1-9).

In some embodiments, administering the effective amount of mosunetuzumab comprises administering mosunetuzumab according to a dosing regimen comprising at least a first dosing cycle and a second dosing cycle, wherein: (a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg (e.g., 0.05 mg to 4.0 mg, 0.1 mg to 4.0 mg, 0.2 mg to 4.0 mg, 0.3 mg to 4.0 mg, 0.4 mg to 4.0 mg, 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.1 mg to 3.5 mg, 0.1 mg to 3.0 mg, 0.1 mg to 2.5 mg, 0.1 mg to 2.0 mg, 0.1 mg to 1.5 mg, 0.1 mg to 1 mg, 0.1 mg to 0.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 0.05 mg to 20.0 mg (e.g., 0.1 mg to 20.0 mg, 0.2 mg to 20.0 mg, 0.3 mg to 20.0 mg, 0.4 mg to 20.0 mg, 0.5 mg to 20.0 mg, 1.0 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 0.5 mg to 15.0 mg, 0.5 mg to 10.0 mg, 0.5 mg to 9.0 mg, 0.5 mg to 8.0 mg, 0.5 mg to 7.0 mg, 0.5 mg to 6.0 mg, 0.5 mg to 5.0 mg, 0.5 mg to 4.0 mg, 0.5 mg to 3.0 mg, 0.5 mg to 2.0 mg, 0.5 mg to 1.0 mg, 1.0 mg to 15.0 mg, 1.0 mg to 10.0 mg, 1.0 mg to 8.0 mg, 1.0 mg to 6.0 mg, 1.0 mg to 4.0 mg, 1.0 mg to 3.0 mg, 10.0 mg to 20.0 mg, 15.0 mg to 20.0 mg, 5.0 mg to 15.0 mg, or 5.0 mg to 10.0 mg; e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg) and the C1D3 is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 3.0 mg to 50.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the second dosing cycle comprises a single dose (C2D1) of mosunetuzumab, wherein the C2D1 is equal to or greater than the C1D3 and is between 0.2 mg to 50 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 3.0 mg to 50.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.4 mg to 4.0 mg (e.g., 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.5 mg to 3.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 1.0 mg to 20.0 mg (e.g., 1.1 mg to 20.0 mg, 1.2 mg to 20.0 mg, 1.3 mg to 20.0 mg, 1.4 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 1.0 mg to 15.0 mg, 1.0 mg to 10.0 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 1.0 mg to 2.0 mg, 10.0 mg to 20.0 mg, 15.0 mg to 20.0 mg, 5.0 mg to 15.0 mg, or 5.0 mg to 10.0 mg; e.g., about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the C2D1 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.8 mg to 3.0 mg (e.g., 0.9 mg to 3.0 mg, 1.0 mg to 3.0 mg, 1.25 mg to 3.0 mg, 1.5 mg to 3.0 mg, 2.0 mg to 3.0 mg, 2.5 mg to 3.0 mg, 0.8 mg to 2.5 mg, 0.8 mg to 2.0 mg, 0.5 mg to 1.5 mg, 0.8 mg to 1.0 mg, 1.0 mg to 3.0 mg, 1.5 mg to 2.0 mg, or 0.9 mg to 1.1 mg; e.g., about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, or about 3.0 mg), the C1D2 is between 1.0 mg to 6.0 mg (e.g., 1.5 mg to 6.0 mg, 2.0 mg to 6.0 mg, 2.5 mg to 6.0 mg, 3.0 mg to 6.0 mg, 3.5 mg to 6.0 mg, 4.0 mg to 6.0 mg, 5.0 mg to 6.0 mg, 5.0 mg to 6.0 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 2.5 mg, 3.0 mg to 6.0 mg, 2.0 mg to 4.0 mg, 2.5 mg to 5.0 mg, or 2.5 mg to 3.5 mg; e.g., about 1.0 mg, about 1.1 mg, about 1.2 mg, about 1.3 mg, about 1.4 mg, about 1.5 mg, about 1.6 mg, about 1.7 mg, about 1.8 mg, 1.9 mg, about 2.0 mg, about 2.1 mg, about 2.2 mg, about 2.3 mg, about 2.4 mg, about 2.5 mg, about. 2.6 mg, about 2.7 mg, about 2.8 mg, about 2.9 mg, about 3.0 mg, about 3.5 mg, about 4.0 mg, about 4.5 mg, about 5.0 mg, about 5.5 mg, or about 6.0 mg), and the C1D3 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg); and (b) the C2D1 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg).

In some embodiments, the C1D1 and C1D2 are each less than the C1D3. In some embodiments, the C1D1 and C1D2 are about equal. In some embodiments, the C1D2 is greater than the C1D1 by about 50% to about 250% (e.g., the C1D2 is greater than the C1D1 by about 50% to about 225%, the C1D2 is greater than the C1D1 by about 50% to about 200%, the C1D2 is greater than the C1D1 by about 50% to about 175%, the C1D2 is greater than the C1D1 by about 50% to about 150%, the C1D2 is greater than the C1D1 by about 50% to about 125%, the C1D2 is greater than the C1D1 by about 50% to about 100%, or the C1D2 is greater than the C1D1 by about 50% to about 75%; e.g., the C1D2 is greater than the C1D1 by about 50%, the C1D2 is greater than the C1D1 by about 100%, the C1D2 is greater than the C1D1 by about 150%, the C1D2 is greater than the C1D1 by about 200%, or the C1D2 is greater than the C1D1 by about 250%).

In some embodiments, (a) the C1D1 is 0.8 mg, the C1D2 is 2.0 mg, and the C1D3 is 4.2 mg, and the C2D1 is 4.2 mg; (b) the C1D1 is 1.0 mg, the C1D2 is 1.0 mg, and the C1D3 is 3.0 mg, and the C2D1 is 30.0 mg; or (c) the C1D1 is 1.0 mg, the C1D2 is 2.0 mg, and the C1D3 is 30.0 mg, and the C2D1 is 30.0 mg.

In some embodiments, the length of the first dosing cycle is 21 days (±1 day). In some embodiments, the method comprises administering to the subject the C1D1, the C1D2, and the C1D3 on or about Days 1, 8 (±1 day), and 15 (±1 day), respectively, of the first dosing cycle. In some embodiments, the length of the second dosing cycle is 28 days (±1 day). In some embodiments, the method comprises administering to the subject the C2D1 on Day 1 of the second dosing cycle.

In some embodiments, the dosing regimen comprises one or more additional dosing cycles (e.g., one dosing cycle, two dosing cycles, three dosing cycles, four dosing cycles, five dosing cycles, six dosing cycles, seven dosing cycles, eight dosing cycles, nine dosing cycles, ten dosing cycles, or more). In some embodiments, the dosing regimen comprises one to ten additional dosing cycles (e.g., one dosing cycle, two dosing cycles, three dosing cycles, four dosing cycles, five dosing cycles, six dosing cycles, seven dosing cycles, eight dosing cycles, nine dosing cycles, or ten dosing cycles). In some embodiments, the dosing regimen comprises ten additional dosing cycles. In some embodiments, the length of each of the one or more additional dosing cycles is 28 days (±1 day). In some embodiments, each of the one or more additional dosing cycles comprises an additional dose of mosunetuzumab. In some embodiments, the method comprises administering to the subject each additional dose of mosunetuzumab on Day 1 of each of the one or more additional dosing cycles. In some embodiments, each additional dose of mosunetuzumab is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, administering the effective amount of mosunetuzumab comprises administering mosunetuzumab according to a dosing regimen comprising twelve dosing cycles, wherein: (a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg (e.g., 0.05 mg to 4.0 mg, 0.1 mg to 4.0 mg, 0.2 mg to 4.0 mg, 0.3 mg to 4.0 mg, 0.4 mg to 4.0 mg, 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.1 mg to 3.5 mg, 0.1 mg to 3.0 mg, 0.1 mg to 2.5 mg, 0.1 mg to 2.0 mg, 0.1 mg to 1.5 mg, 0.1 mg to 1 mg, 0.1 mg to 0.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 0.05 mg to 20.0 mg (e.g., 0.1 mg to 20.0 mg, 0.2 mg to 20.0 mg, 0.3 mg to 20.0 mg, 0.4 mg to 20.0 mg, 0.5 mg to 20.0 mg, 1.0 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 0.5 mg to 15.0 mg, 0.5 mg to 10.0 mg, 0.5 mg to 9.0 mg, 0.5 mg to 8.0 mg, 0.5 mg to 7.0 mg, 0.5 mg to 6.0 mg, 0.5 mg to 5.0 mg, 0.5 mg to 4.0 mg, 0.5 mg to 3.0 mg, 0.5 mg to 2.0 mg, 0.5 mg to 1.0 mg, 1.0 mg to 15.0 mg, 1.0 mg to 10.0 mg, 1.0 mg to 8.0 mg, 1.0 mg to 6.0 mg, 1.0 mg to 4.0 mg, 1.0 mg to 3.0 mg, 10.0 mg to 20.0 mg, 15.0 mg to 20.0 mg, 5.0 mg to 15.0 mg, or 5.0 mg to 10.0 mg; e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 0.2 mg to 50.0 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) the second to twelfth dosing cycles each comprises a single dose (C2D1-C12D1) of mosunetuzumab, wherein each single dose C2D1-C12D1 are equivalent in amount, is equal to or greater than the C1D3, and is between 0.2 mg to 50 mg (e.g., 0.3 mg to 50.0 mg, 0.4 mg to 50.0 mg, 0.5 mg to 50.0 mg, 1.0 mg to 50.0 mg, 2.0 mg to 50.0 mg, 3.0 mg to 50.0 mg, 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 1.0 mg to 45.0 mg, 1.0 mg to 40.0 mg, 1.0 mg to 35.0 mg, 1.0 mg to 30.0 mg, 1.0 mg to 25.0 mg, 1.0 mg to 20.0 mg, 1.0 mg to 15.0 mg, 1.0 mg to 10.0 mg, 1.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 3.0 mg to 45.0 mg, 4.0 mg to 4.5 mg, 3.0 mg to 10.0 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 0.2 mg, about 0.5 mg, about 0.8 mg, about 1.0 mg, about 1.5 mg, about 2.0 mg, about 2.5 mg, about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.4 mg to 4.0 mg (e.g., 0.5 mg to 4.0 mg, 0.75 mg to 4.0 mg, 1.0 mg to 4.0 mg, 1.25 mg to 4.0 mg, 1.5 mg to 4.0 mg, 2.0 mg to 4.0 mg, 2.5 mg to 4.0 mg, 3.0 mg to 4.0 mg, 3.5 mg to 4.0 mg, 0.5 mg to 3.5 mg, 0.5 mg to 3.0 mg, 0.5 mg to 2.0 mg, 0.5 mg to 1.0 mg, 0.75 mg to 1.25 mg, 1.0 mg to 3.0 mg, 1.5 mg to 2.0 mg, 0.7 mg to 0.9 mg, or 0.9 mg to 1.1 mg; e.g., about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, about 3.0 mg, about 3.5 mg, or about 4.0 mg), the C1D2 is between 1.0 mg to 20.0 mg (e.g., 1.1 mg to 20.0 mg, 1.2 mg to 20.0 mg, 1.3 mg to 20.0 mg, 1.4 mg to 20.0 mg, 1.5 mg to 20.0 mg, 2.0 mg to 20.0 mg, 3.0 mg to 20.0 mg, 4.0 mg to 20.0 mg, 5.0 mg to 20.0 mg, 6.0 mg to 20.0 mg, 7.0 mg to 20.0 mg, 8.0 mg to 20.0 mg, 9.0 mg to 20.0 mg, 10.0 mg to 20.0 mg, 15.0 mg to 20.0 mg, 1.0 mg to 15.0 mg, 1.0 mg to 10.0 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 1.0 mg to 2.0 mg, 10.0 mg to 20.0 mg, 15.0 mg to 20.0 mg, 5.0 mg to 15.0 mg, or 5.0 mg to 10.0 mg; e.g., about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 5.0 mg, about 6.0 mg, about 7.0 mg, about 8.0 mg, about 9.0 mg, about 10.0 mg, about 15.0 mg, or about 20.0 mg), and the C1D3 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg); and (b) each single dose C2D1-C12D1 is between 3.0 mg to 50.0 mg (e.g., 4.0 mg to 50.0 mg, 5.0 mg to 50.0 mg, 10.0 mg to 50.0 mg, 15.0 mg to 50.0 mg, 20.0 mg to 50.0 mg, 25.0 mg to 50.0 mg, 30.0 mg to 50.0 mg, 35.0 mg to 50.0 mg, 40.0 mg to 50.0 mg, 45.0 mg to 50.0 mg, 3.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 25.0 mg to 50.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, about 45.0 mg, or about 50.0 mg).

In some embodiments, (a) the C1D1 is between 0.8 mg to 3.0 mg (e.g., 0.9 mg to 3.0 mg, 1.0 mg to 3.0 mg, 1.25 mg to 3.0 mg, 1.5 mg to 3.0 mg, 2.0 mg to 3.0 mg, 2.5 mg to 3.0 mg, 0.8 mg to 3.0 mg, 0.8 mg to 2.5 mg, 0.8 mg to 2.0 mg, 0.5 mg to 1.5 mg, 0.8 mg to 1.0 mg, 1.0 mg to 3.0 mg, 1.5 mg to 2.0 mg, or 0.9 mg to 1.1 mg; e.g., about 0.8 mg, about 0.9 mg, about 1.0 mg, about 1.1 mg, about 1.2 mg, about 1.3 mg, about 1.4 mg, about 1.5 mg, about 1.75 mg, about 2.0 mg, about 2.25 mg, about 2.5 mg, about 2.75 mg, or about 3.0 mg), the C1D2 is between 1.0 mg to 6.0 mg (e.g., 1.5 mg to 6.0 mg, 2.0 mg to 6.0 mg, 2.5 mg to 6.0 mg, 3.0 mg to 6.0 mg, 3.5 mg to 6.0 mg, 4.0 mg to 6.0 mg, 5.0 mg to 6.0 mg, 5.0 mg to 6.0 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 2.5 mg, 3.0 mg to 6.0 mg, 2.0 mg to 4.0 mg, 2.5 mg to 5.0 mg, or 2.5 mg to 3.5 mg; e.g., about 1.0 mg, about 1.1 mg, about 1.2 mg, about 1.3 mg, about 1.4 mg, about 1.5 mg, about 1.6 mg, about 1.7 mg, about 1.8 mg, 1.9 mg, about 2.0 mg, about 2.1 mg, about 2.2 mg, about 2.3 mg, about 2.4 mg, about 2.5 mg, about. 2.6 mg, about 2.7 mg, about 2.8 mg, about 2.9 mg, about 3.0 mg, about 3.5 mg, about 4.0 mg, about 4.5 mg, about 5.0 mg, about 5.5 mg, or about 6.0 mg), and the C1D3 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg); and (b) each single dose C2D1-C12D1 is between 3.0 mg to 45.0 mg (e.g., 4.0 mg to 45.0 mg, 5.0 mg to 45.0 mg, 10.0 mg to 45.0 mg, 15.0 mg to 45.0 mg, 20.0 mg to 45.0 mg, 25.0 mg to 45.0 mg, 30.0 mg to 45.0 mg, 35.0 mg to 45.0 mg, 40.0 mg to 45.0 mg, 3.0 mg to 40.0 mg, 3.0 mg to 35.0 mg, 3.0 mg to 30.0 mg, 3.0 mg to 25.0 mg, 3.0 mg to 20.0 mg, 3.0 mg to 15.0 mg, 3.0 mg to 10.0 mg, 3.0 mg to 5.0 mg, 10.0 mg to 20.0 mg, 20.0 mg to 30.0 mg, 30.0 mg to 40.0 mg, 5.0 mg to 25.0 mg, 4.0 mg to 4.5 mg, 4.0 mg to 5.0 mg, 25.0 mg to 35.0 mg, 28.0 mg to 32.0 mg, 10.0 mg to 30.0 mg, or 20.0 mg to 40.0 mg; e.g., about 3.0 mg, about 4.0 mg, about 4.2 mg, about 4.5 mg, about 5.0 mg, about 5.5 mg, about 6.0 mg, about 6.5 mg, about 7.0 mg, about 8.0 mg, about 9.0 mg, about 10.0 mg, about 12.0 mg, about 15.0 mg, about 20.0 mg, about 25.0 mg, about 30.0 mg, about 35.0 mg, about 40.0 mg, or about 45.0 mg).

In some embodiments, the C1D1 and C1D2 are each less than the C1D3. In some embodiments, the C1D2 is greater than the C1D1 by about 50% to about 250% (e.g., the C1D2 is greater than the C1D1 by about 50% to about 225%, the C1D2 is greater than the C1D1 by about 50% to about 200%, the C1D2 is greater than the C1D1 by about 50% to about 175%, the C1D2 is greater than the C1D1 by about 50% to about 150%, the C1D2 is greater than the C1D1 by about 50% to about 125%, the C1D2 is greater than the C1D1 by about 50% to about 100%, or the C1D2 is greater than the C1D1 by about 50% to about 75%; e.g., the C1D2 is greater than the C1D1 by about 50%, the C1D2 is greater than the C1D1 by about 100%, the C1D2 is greater than the C1D1 by about 150%, the C1D2 is greater than the C1D1 by about 200%, or the C1D2 is greater than the C1D1 by about 250%).

In some embodiments, (a) the C1D1 is 0.8 mg, the C1D2 is 2.0 mg, the C1D3 is 4.2 mg, and each single dose C2D1-C12D1 is 4.2 mg; (b) the C1D1 is 1.0 mg, the C1D2 is 1.0 mg, the C1D3 is 3.0 mg, and each single dose C2D1-C12D1 is 30.0 mg; or (c) the C1D1 is 1.0 mg, the C1D2 is 2.0 mg, the C1D3 is 30.0 mg, and each single dose C2D1-C12D1 is 30.0 mg.

In some embodiments, the length of the first dosing cycle is 21 days (±1 day). In some embodiments, the method comprises administering to the subject the C1D1, the C1D2, and the C1D3 on or about Days 1, 8 (±1 day), and 15 (±1 day), respectively, of the first dosing cycle. In some embodiments, the length of each of the second to twelfth dosing cycles is 28 days (±1 day). In some embodiments, the method comprises administering to the subject each of the C2D1-C12D1 on Day 1 of each respective dosing cycle. In some embodiments, the length of each of the second to twelfth dosing cycles is 28 days (±1 day).

In some embodiments, mosunetuzumab is administered intravenously.

In some embodiments, lenalidomide is administered during the second and subsequent cycles. In some embodiments, lenalidomide is not administered during the first cycle. In some embodiments, lenalidomide is administered daily. In some embodiments, lenalidomide is administered daily on the first 21 days of each dosing cycle comprising administration of lenalidomide. In some embodiments, lenalidomide is not administered on the last 7 days of each dosing cycle comprising administration of lenalidomide. In some embodiments, lenalidomide is administered at a dose of 20 mg. In some embodiments, lenalidomide is administered orally.

In some embodiments, the subject was previously treated with at least one anti-CD20 monoclonal antibody. In some embodiments, the subject is relapsed or refractory to the treatment comprising the anti-CD20 monoclonal antibody. In some embodiments, the anti-CD20 monoclonal antibody is obinutuzumab or rituximab.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included obinutuzumab; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-21 of the second dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included obinutuzumab; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprise a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included rituximab; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab administered on Day 1 of the second dosing cycle, wherein the C2D1 is 30 mg, and (c) the second dosing cycle further comprises administering 20 mg lenalidomide daily orally on Days 1-21 of the second dosing cycle.

In one aspect, the invention features a method of treating a subject, comprising administering to the subject an effective amount of mosunetuzumab and an effective amount of lenalidomide, wherein the subject: (i) has relapsed or refractory follicular lymphoma (R/R FL) that expresses CD20, and (ii) has been previously treated with at least one chemo-immunotherapy regimen that included rituximab; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprises a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle.

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-21 of the second dosing cycle. In some embodiments, at least one chemo-immunotherapy regimen included an anti-CD20 monoclonal antibody (e.g., rituximab or obinutuzumab).

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprises a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle. In some embodiments, at least one chemo-immunotherapy regimen included an anti-CD20 monoclonal antibody (e.g., rituximab or obinutuzumab).

In some embodiments, the dosing regimen further comprises administration of a corticosteroid. In some embodiments, the corticosteroid is administered to the subject during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subject on Days 1, 8 (±1 day), and 15 (±1 day), respectively, of the first dosing cycle. In some embodiments, wherein each single dose C1D1-C1D3 of the corticosteroid is administered to the subject before administration of the C1D1-C1D3, respectively, of mosunetuzumab. In some embodiments, each single dose C1D1-C1D3 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab.

In some embodiments, the corticosteroid is administered to the subject on the first dosing cycle and not on the second dosing cycle.

In some embodiments, the corticosteroid is administered to the subject on the second dosing cycle. In some embodiments, the second dosing cycle comprises a single dose (C2D1) of the corticosteroid. In some embodiments, the C2D1 of the corticosteroid is administered to the subject on Day 1 of the second dosing cycle. In some embodiments, the C2D1 of the corticosteroid is administered to the subject before administration of the C2D1 of mosunetuzumab. In some embodiments, the C2D1 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, the dosing regimen further comprises administration of a corticosteroid. In some embodiments, the corticosteroid is administered to the subjects during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subjects on Days 1, 8 (±1 day), and 15 (±1 day), respectively, of the first dosing cycle. In some embodiments, each single dose C1D1-C1D3 of the corticosteroid is administered to the subjects before administration of the C1D1-C1D3, respectively, of mosunetuzumab. In some embodiments, each single dose C1D1-C1D3 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab.

In some embodiments, the corticosteroid is administered to the subjects on the first dosing cycle and not on the second dosing cycle.

In some embodiments, the corticosteroid is administered to the subjects on the second dosing cycle. In some embodiments, the second dosing cycle comprises a single dose (C2D1) of the corticosteroid. In some embodiments, the C2D1 of the corticosteroid is administered to the subjects on Day 1 of the second dosing cycle. In some embodiments, the C2D1 of the corticosteroid is administered to the subjects before administration of the C2D1 of mosunetuzumab. In some embodiments, the C2D1 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, each additional dosing cycle comprises administering to the subject an additional dose of the corticosteroid. In some embodiments, each additional dose of the corticosteroid is administered on Day 1 of each additional dosing cycle. In some embodiments, each additional dose of the corticosteroid is administered to the subject before administration of each additional dose of mosunetuzumab. In some embodiments, each additional dose of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of each additional dose of mosunetuzumab.

In some embodiments, the corticosteroid is administered intravenously. In some embodiments, the corticosteroid is dexamethasone. In some embodiments, each dose of dexamethasone is about 10 mg (e.g., about 1, 2, 3, 4, 5, 6, 7, 8, 9, 10, 11, 12, 13, 14, 15, 16, 17, 18, 19, or 20 mg).

In some embodiments, the overall response rate in a population of subjects having R/R FL is at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more; e.g., between 80%-100%, between 85-100%, between 87-100%, between 90-100%, between 95-100%, between 80%-90%, between 80-85%, between 85-97%, between 85-95%, between 85-90%, between 85-87%, between 90-95%, or between 93-97%; e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more). In some embodiments, the overall response rate in a population of subjects having R/R FL is at least 90%. In some embodiments, the overall response rate in a population of subjects having R/R FL is at least 95%. In some embodiments, the overall response rate in a population of subjects having R/R FL is at least 99%.

In some embodiments, the complete response rate in a population of subjects having R/R FL is at least 65% (e.g., at least 70%, at least 75%, at least 80%, at least 85%, at least 90%, at least 95%, or more; e.g., between 65-100%, between 75-100%, between 85-100%, between 95-100%, between 65-90%, between 65-80%, between 65-70%, between 65-75%, or between 75-85%; e.g., about 65%, about 66%, about 67%, about 68%, about 69%, about 70%, about 71%, about 72%, about 73%, about 74%, about 75%, about 76%, about 77%, about 80%, about 85%, about 90%, or more). In some embodiments, the complete response rate in a population of subjects having R/R FL is at least 75%. In some embodiments, the complete response rate in a population of subjects having R/R FL is at least 85%.

In some embodiments, combination therapy with mosunetuzumab and lenalidomide exhibits synergistic effects in the efficacy of the therapy compared to therapy with mosunetuzumab or lenalidomide alone for treating subjects with R/R FL. In some embodiments, a population of subjects with R/R FL treated with a combination therapy with mosunetuzumab and lenalidomide exhibits a higher overall response rate compared to a population of subjects treated with monotherapy of mosunetuzumab or lenalidomide alone. In some embodiments, a population of subjects with R/R FL treated with a combination therapy with mosunetuzumab and lenalidomide exhibits a higher complete response rate compared to a population of subjects treated with monotherapy of mosunetuzumab or lenalidomide alone.

In some embodiments, the subject exhibits a reduction in tumor burden after being administered an effective amount of mosunetuzumab and an effective amount of lenalidomide. In some embodiments, the reduction in tumor burden is determined by computed tomography (CT). In some embodiments, the reduction in tumor burden is a decrease in the sum of the product of the diameters (SPD) of target lesions. In some embodiments, the decrease in SPD is at least 40% (e.g., at least 50%, at least 60%, at least 70%, at least 80%, at least 90%, at least 95%, at least 96%, at least 97%, at least 98%, at least 99%, or more; e.g., between 40-100%, between 40-80%, between 40-60%, between 50-70%, between 70-90%, between 75-85%, between 60-100%, between 55-65%, between 80-100%; e.g., about 40%, about 45%, about 50%, about 55%, about 60%, about 65%, about 70%, about 75%, about 80%, about 85%, about 90%, about 95%, or about 100%). In some embodiments, the decrease in SPD is at least 60%. In some embodiments, the decrease in SPD is at least 80%.

In some embodiments, at least 45% (e.g., at least 50%, at least 60%, at least 70%, at least 80%, at least 90%, at least 95%, at least 96%, at least 97%, at least 98%, at least 99%, or more; e.g., between 45-100%, between 45-80%, between 45-60%, between 50-70%, between 55-65%, between 70-90%, between 70-80%, between 60-100%, between 80-100%; e.g., about 45%, about 50%, about 55%, about 60%, about 65%, about 70%, about 75%, about 80%, about 85%, about 90%, about 95%, or about 100%) of subjects in the population exhibit a reduction in tumor burden after being administered an effective amount of mosunetuzumab and an effective amount of lenalidomide. In some embodiments, at least 60% of subjects in the population exhibit a reduction in tumor burden after being administered an effective amount of mosunetuzumab and an effective amount of lenalidomide. In some embodiments, at least 75% of subjects in the population exhibit a reduction in tumor burden after being administered an effective amount of mosunetuzumab and an effective amount of lenalidomide. In some embodiments, the reduction in tumor burden is determined by computed tomography (CT).

In some embodiments, the reduction in tumor burden is a decrease in the sum of the product of the diameters (SPD) of target lesions. In some embodiments, the decrease in SPD is at least 40% (e.g., at least 50%, at least 60%, at least 70%, at least 80%, at least 90%, at least 95%, at least 96%, at least 97%, at least 98%, at least 99%, or more; e.g., between 40-100%, between 40-80%, between 40-60%, between 50-70%, between 70-90%, between 75-85%, between 60-100%, between 55-65%, between 80-100%; e.g., about 40%, about 45%, about 50%, about 55%, about 60%, about 65%, about 70%, about 75%, about 80%, about 85%, about 90%, about 95%, or about 100%). In some embodiments, the decrease in SPD is at least 60%. In some embodiments, the decrease in SPD is at least 80%.

In some embodiments, the subject is human. In some embodiments, each subject in the population is human.

### B. Dosing Strategies for Mitigating Adverse Events

The present invention relates to methods of treating a subject having a relapsed and/or refractory (R/R) follicular lymphoma (FL) by administration of mosunetuzumab and lenalidomide as a combination therapy. In particular, the present invention relates to methods of treating a subject having a R/R FL that is R/R to at least one prior systemic therapy (e.g., one prior treatment with a chemo-immunotherapy regimen) which included an anti-CD20 antibody (e.g., an anti-CD20 monoclonal antibody; e.g., rituximab or obinutuzumab) or other treatment. The therapies and dosing regimens described herein provide acceptable safety profiles in subjects with R/R FL treated with the described dosing regimens.

1. CRS Symptoms and Grading

Any of the methods described herein may involve monitoring a subject for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biol Blood Marrow Transplantation. 25(4): 625-638, 2019). The ASTCT criteria is intended to be objective, easy to apply, and more accurately categorize the severity of CRS. This CRS grading system is shown below in Table 1.

TABLE 1
CRS Grading System

| CRS | | | | |
|---|---|---|---|---|
| Parameter | Grade 1 | Grade 2 | Grade 3 | Grade 4 |
| Fever | Temperature ≥ | Temperature ≥ | Temperature ≥ | Temperature ≥ |
| | 38° C. | 38° C. | 38° C. | 38° C. |

with

| | | | | |
|---|---|---|---|---|
| Hypotension | None | Not requiring | Requiring a | Requiring multiple |
| | | vasopressors | vasopressor with or | vasopressors |
| | | | without vasopressin | (excluding |
| | | | | vasopressin) |

and/or

| | | | | |
|---|---|---|---|---|
| Hypoxia | None | Requiring low-glow | Requiring high-flow | Requiring positive |
| | | nasal cannula or | nasal cannula, | pressure |
| | | blow-by | facemask, | (e.g., CPAP, BiPAP, |
| | | | nonrebreather mask | intubation and |
| | | | or Venturi mask | mechanical |
| | | | | ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events.

Fever is defined as a temperature 38° C. not attributable to any other cause. In subjects who have CRS then receive antipyretic or anticytokine therapy such as tocilizumab or steroids, fever is no longer required to grade subsequent CRS severity. In this case, CRS grading is determined by hypotension and/or hypoxia.

CRS grade is determined by the more severe event, hypotension or hypoxia not attributable to any other cause. For example, a subject with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25(1): 31-5, 2004; Lee et al. Blood. 124(2): 188-95, 2014); Doesegger et al. Clin. Transl. Immunology. 4(7): e39, 2015), and IL-6 correlates with the severity of CRS, with subjects who experience a Grade 4 or 5 CRS event having much higher IL-6 levels compared to subjects who do not experience CRS or experience milder CRS (Grades 0-3) (Chen et al. J. Immunol. Methods. 434:1-8, 2016).

Therefore, blocking the inflammatory action of IL-6 using an agent that inhibits IL-6-mediated signaling to manage CRS observed in subjects during the double-step fractionated, dose-escalation dosing regimen is an alternative to steroid treatment that would not be expected to negatively impact T cell function or diminish the efficacy or clinical benefit of mosunetuzumab therapy in the treatment of CD20-positive cell proliferative disorders (e.g., a B cell proliferative disorders).

If the subject has a CRS event that does not resolve or worsens within 24 hours of administering the IL-6R antagonist to treat the symptoms of the CRS event, and the method may further comprise administering to the subject one or more additional doses of the IL-6R antagonist to manage the CRS event. The subject may be administered a corticosteroid, such as methylprednisolone or dexamethasone if CRS event is not managed through administration of the IL-6R antagonist.

2. Other Adverse Events and Grading

Any of the methods described herein may involve monitoring a subject for additional non-CRS adverse events. Incidence, nature, and severity of physical findings and adverse events, with severity determined according to the National Cancer Institute Common Terminology Criteria for Adverse Events version 5 (NCI CTCAE v5.0). Other than CRS, one of the most common adverse events reported in patients undergoing treatment with mosunetuzumab and/or lenalidomide is neutropenia (e.g., febrile neutropenia).

Neutropenia is characterized by an abnormally low blood count of neutrophils, which are a type of white blood cells. Neutropenia may lead to an increased risk of infection. The generally accepted reference range for absolute neutrophil count (ANC) in adult humans is 1,500 to 8,000 cells/μL of blood. Mild neutropenia is characterized by ANC between 1,000-1,500 cells/μL (Grade 1-2); moderate neutropenia is characterized by ANC between 500 and 1,000 cells/μL (Grade 3), and severe neutropenia is characterized by ANC below 500 cells/μL (Grade 4). Febrile neutropenia (Grade 3+ neutropenia) is characterized by ANC below 1,000 cells/μL in addition to either a single temperature measurement greater than 38.3° C. or sustained temperature measurements greater than 38° C. for more than one hour.

3. Dosing Regimens with Acceptable Safety Profiles

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising at least a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-21 of the second dosing cycle. In some embodiments, at least one chemo-immunotherapy regimen included an anti-CD20 monoclonal antibody (e.g., rituximab or obinutuzumab).

In one aspect, the invention features a method of treating a population of subjects, comprising administering to each subject in the population an effective amount of mosunetuzumab and an effective amount of lenalidomide, wherein each subject: (a) has relapsed or refractory follicular lymphoma (R/R FL) that expresses CD20, and (b) has been previously treated with at least one chemo-immunotherapy regimen; and wherein administering the effective amount of mosunetuzumab and lenalidomide comprises administering mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab intravenously administered on Days 1, 8 (±1 day), and 15 (±1 day), respectively, of the first dosing cycle, wherein the C1D1 is 1 mg, the C1D2 is 2 mg, and the C1D3 is 30 mg, (b) the second to twelfth dosing cycles each comprises a single dose (C2D1-C12D1) of mosunetuzumab intravenously administered on Day 1 of each dosing cycle, wherein each single dose C2D1-C12D1 is 30 mg, and (c) the second to twelfth dosing cycles each further comprises orally administering 20 mg lenalidomide daily on Days 1-21 of each dosing cycle. In some embodiments, at least one chemo-immunotherapy regimen included an anti-CD20 monoclonal antibody (e.g., rituximab or obinutuzumab).

In some embodiments, each subject has received only one prior line of systemic therapy and either: (a) has a Follicular Lymphoma International Prognostic Index (FLIPI; Solal-Céligny et al. Blood. 2004; 104 (5): 1258-1265.) score of 2-5 (e.g., a score of 2, 3, 4, or 5), (b) has been refractory to prior anti-CD20 monoclonal antibody treatment, or (c) has disease progression within 24 months after initiation of prior therapy.

In some embodiments, the FL of each subject is histologically documented as Grade 1, 2, or 3a, but not 3b according to the World Health Organization classification of lymphoid neoplasms (as referenced in Swerdlow S H, et al. Blood 2016; 127:2375-90).

In some embodiments, each subject has not been treated with the anti-CD20 monoclonal antibody for at least 4 weeks (e.g., 4 weeks, 6 weeks, 8 weeks, 10 weeks, 12 weeks, 24 weeks, 36 weeks, 48 weeks, 60 weeks, or more) prior to being administered the effective amount of mosunetuzumab and lenalidomide.

In some embodiments, mosunetuzumab and lenalidomide have a synergistic effect on the R/R FL. In some embodiments, the synergistic effect is a partial response, as defined by Lugano 2014 criteria (Cheson B D, et al. J Clin Oncol 2014; 32:1-9). In some embodiments, the synergistic effect is a complete response, as defined by Lugano 2014 criteria (Cheson B D, et al. J Clin Oncol 2014; 32:1-9).

In one aspect, the methods of the invention features premedication with corticosteroids to reduce adverse effects of mosunetuzumab administration. In some embodiments, premedication with corticosteroids reduces the rate of cytokine release syndrome (CRS) in subjects treated with mosunetuzumab (e.g., subjects administered a combination of mosunetuzumab and lenalidomide).

In some embodiments, the dosing regimen further comprises administration of a corticosteroid. In some embodiments, the corticosteroid is administered to the subject during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subject on Days 1, 8 (±1 day), and 15 (±1 day), respectively, of the first dosing cycle. In some embodiments, wherein each single dose C1D1-C1D3 of the corticosteroid is administered to the subject before administration of the C1D1-C1D3, respectively, of mosunetuzumab. In some embodiments, each single dose C1D1-C1D3 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab. In some embodiments, the corticosteroid used is dexamethasone. In some embodiments, each dose of dexamethasone is about 10 mg (e.g., about 1, 2, 3, 4, 5, 6, 7, 8, 9, 10, 11, 12, 13, 14, 15, 16, 17, 18, 19, or 20 mg). In some embodiments, dexamethasone is administered intravenously.

In some embodiments, the corticosteroid is administered to the subject on the first dosing cycle and not on the second dosing cycle.

In some embodiments, the corticosteroid is administered to the subject on the second dosing cycle. In some embodiments, the second dosing cycle comprises a single dose (C2D1) of the corticosteroid. In some embodiments, the C2D1 of the corticosteroid is administered to the subject on Day 1 of the second dosing cycle. In some embodiments, the C2D1 of the corticosteroid is administered to the subject before administration of the C2D1 of mosunetuzumab. In some embodiments, the C2D1 of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, the dosing regimen further comprises administration of a corticosteroid. In some embodiments, the corticosteroid is administered to the subjects during the first dosing cycle. In some embodiments, the first dosing cycle comprises a first dose (C1D1), a second dose (C1D2), and a third dose (C1D3) of the corticosteroid. In some embodiments, the C1D1, the C1D2, and the C1D3 of the corticosteroid are administered to the subjects on Days 1, 8 (±1 day), and 15 (±1 day), respectively, of the first dosing cycle. In some embodiments, each single dose C1D1-C1D3 of the corticosteroid is administered to the subjects before administration of the C1D1-C1D3, respectively, of mosunetuzumab. In some embodiments, each single dose C1D1-C1D3 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C1D1-C1D3, respectively, of mosunetuzumab.

In some embodiments, the corticosteroid is administered to the subjects on the first dosing cycle and not on the second dosing cycle.

In some embodiments, the corticosteroid is administered to the subjects on the second dosing cycle. In some embodiments, the second dosing cycle comprises a single dose (C2D1) of the corticosteroid. In some embodiments, the C2D1 of the corticosteroid is administered to the subjects on Day 1 of the second dosing cycle. In some embodiments, the C2D1 of the corticosteroid is administered to the subjects before administration of the C2D1 of mosunetuzumab. In some embodiments, the C2D1 of the corticosteroid is administered to the subjects 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of the C2D1 of mosunetuzumab.

In some embodiments, each additional dosing cycle comprises administering to the subject an additional dose of the corticosteroid. In some embodiments, each additional dose of the corticosteroid is administered on Day 1 of each additional dosing cycle. In some embodiments, each additional dose of the corticosteroid is administered to the subject before administration of each additional dose of mosunetuzumab. In some embodiments, each additional dose of the corticosteroid is administered to the subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of each additional dose of mosunetuzumab.

In some embodiments, the corticosteroid is administered intravenously. In some embodiments, the corticosteroid is dexamethasone. In some embodiments, each dose of dexamethasone is about 10 mg (e.g., about 1, 2, 3, 4, 5, 6, 7, 8, 9, 10, 11, 12, 13, 14, 15, 16, 17, 18, 19, or 20 mg).

In some embodiments, the incidence of an adverse event is not significantly higher than if mosunetuzumab was administered alone to the population of subjects having R/R FL. In some embodiments, the incidence of an adverse event is not significantly higher than if lenalidomide is not administered to the population of subjects. In some embodiments, the incidence rate of an adverse event is not significantly higher in a population of subjects having R/R FL when treated with a combination therapy of mosunetuzumab and lenalidomide in comparison to a population of subjects having R/R FL treated with a monotherapy of mosunetuzumab or lenalidomide alone.

In some embodiments, the incidence rate of cytokine release syndrome as defined by the American Society of Transplantation and Cellular Therapy (ASTCT) Consensus Grading for Cytokine-Release Syndrome (“ASTCT CRS grading;” Lee et al., Biol Blood Marrow Transplant 2019) is less than 45% (e.g., less than 40%, less than 35%, less than 30%, less than 25%, less than 20%, less than 15%, less than 10%, less than 5%, less than 3%, or less than 1%; e.g., between about 0% to about 50%, between about 5% to about 40%, between about 5% to about 20%, between about 5% to about 10%, between about 20% to about 45%, between about 30% to about 40%, between about 20% to about 40%, between about 15% to about 35%, between about 15% to about 25%, between about 35% to about 45%, or between about 25% to about 45%; e.g., about 45%, about 40%, about 35%, about 30%, about 25%, about 20%, about 15%, about 10%, about 5%, about 1%, or about 0%). In some embodiments, the incidence rate of cytokine release syndrome as defined by the American Society of Transplantation and Cellular Therapy (ASTCT) Consensus Grading for Cytokine-Release Syndrome (“ASTCT CRS grading”) is less than 35%. In some embodiments, the incidence rate of cytokine release syndrome as defined by the American Society of Transplantation and Cellular Therapy (ASTCT) Consensus Grading for Cytokine-Release Syndrome (“ASTCT CRS grading”) is less than 25%. In some embodiments, the incidence rate of CRS is about 30%.

In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 10% (e.g., less than 9%, less than 8%, less than 7%, less than 6%, less than 5%, less than 4%, less than 3%, less than 2%, less than 1%; e.g., between 0% and 10%, between 0% and 9%, between 0% and 8%, between 0% and 7%, between 0% and 6%, between 0% and 5%, between 0% and 4%, between 0% and 3%, between 0% and 2%, between 0% and 1%, between 1% and 3%, between 1% and 5%, between 1% and 10%, between 3% and 5%, between 5% and 8%, between 5% and 10%, or between 8% and 10%). In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 5%. In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 3%. In some embodiments, the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 1%. In some embodiments, the incidence rate of CRS having a grade of 3 or higher is about 0%.

In some embodiments, the incidence rate of neutropenia in a population of patients having R/R FL and treated with combination therapy of mosunetuzumab and lenalidomide is less than 40% (e.g., less than 35%, less than 30%, less than 25%, less than 20%, less than 15%, less than 10%, less than 5%, less than 3%, or less than 1%; e.g., between about 0% to about 40%, between about 5% to about 40%, between about 5% to about 20%, between about 5% to about 10%, between about 20% to about 40%, between about 30% to about 40%, between about 15% to about 35%, between about 15% to about 25%, between about 35% to about 40%, or between about 25% to about 40%; e.g., about 40%, about 35%, about 30%, about 25%, about 20%, about 15%, about 10%, about 5%, about 3%, about 1%, or about 0%). In some embodiments, the incidence rate of neutropenia is less than 30%. In some embodiments, the incidence rate of neutropenia is less than 20%. In some embodiments, the incidence rate of neutropenia having Grade of 3 or 4 is about 19%. In some embodiments, the incidence rate of febrile neutropenia is about 0%.

The methods described herein may result in an acceptable safety profile for subjects having R/R mosunetuzumab being treated with combination therapy of mosunetuzumab and lenalidomide. In some instances, treatment using the methods described herein that result in intravenously administering mosunetuzumab in the context of a fractionated, dose-escalation dosing regimen and oral administration of lenalidomide results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or hepatotoxicities, following treatment with mosunetuzumab using the fractionated, dose-escalation dosing regimen of the invention relative to treatment with mosunetuzumab using a non-fractioned dosing regimen.

### IV. Therapeutic Agents

### A. Mosunetuzumab

The invention provides mosunetuzumab, a bispecific antibody that binds to CD20 and CD3, useful for treating relapsed and/or refractory (R/R) follicular lymphoma (FL). The FL may be of Grades 1, 2, or 3a, but not Grade 3b.

In some instances, the invention provides mosunetuzumab that includes an anti-CD20 arm having a first binding domain comprising at least one, two, three, four, five, or six hypervariable regions (HVRs) selected from (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). In some instances, mosunetuzumab comprises at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively. In some instances, mosunetuzumab comprises an anti-CD20 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO: 8.

In some instances, the invention provides mosunetuzumab that includes an anti-CD3 arm having a second binding domain comprising at least one, two, three, four, five, or six HVRs selected from (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). In some instances, mosunetuzumab comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, mosunetuzumab comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, the invention provides mosunetuzumab that includes (1) an anti-CD20 arm having a first binding domain comprising at least one, two, three, four, five, or six HVRs selected from (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6); and (2) an anti-CD3 arm having a second binding domain comprising at least one, two, three, four, five, or six HVRs selected from (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). In some instances, mosunetuzumab comprises (1) at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, mosunetuzumab comprises (1) an anti-CD20 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, mosunetuzumab comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, mosunetuzumab has the International Nonproprietary Names for Pharmaceutical Substances (INN) List 117 (WHO Drug Information, Vol. 31, No. 2, 2017, p. 303), or CAS Registry No. 1905409-39-3, and having (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. In some instances, mosunetuzumab comprises (1) an anti-CD20 arm comprising a first binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 33; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 34; or (c) a heavy chain as in (a) and a light chain as in (b), and (2) an anti-CD3 arm comprising a second binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 36; or (c) a heavy chain as in (a) and a light chain as in (b). In some instances, mosunetuzumab comprises (1) an anti-CD20 arm comprising a first binding domain comprising a heavy chain comprising an amino acid sequence of SEQ ID NO: 33 and a light chain comprising an amino acid sequence of SEQ ID NO: 34 and (2) an anti-CD3 arm comprising a second binding domain comprising a heavy chain comprising an amino acid sequence of SEQ ID NO: 35 and a light chain comprising an amino acid sequence of SEQ ID NO: 36.

Amino acid sequences of mosunetuzumab are summarized in Table 2 below.

TABLE 2
Sequence IDs for Mosunetuzumab

| CD3 Arm | | CD20 Arm | |
|---|---|---|---|
| SEQ ID NO: | Description | SEQ ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Lenalidomide

Lenalidomide is an immunomodulatory (IMiD) imide drug that binds to cereblon, an E3 ubiquitin ligase protein (Gribben et al. 2015). The immunomodulatory activity of lenalidomide is not completely understood; however, lenalidomide has been shown to enhance CD4+ and CD8+ T-cell co-stimulation, induce T-cell proliferation, and enhance IL-2 and IFN-γ (Haslett et al. 1998; Davies et al. 2001).

Lenalidomide has the CAS Registry Number 191732-72-6 and IUPAC name (3RS)-3-(4-Amino-1-oxo-1,3-dihydro-2H-isoindol-2-yl)piperidine-2,6-dione. Lenalidomide is also known by tradenames including REVLIMID®, linamid, and lenalid. Lenalidomide has the DrugBank Accession Number DB00480, PubChem CID 216326, and chemical formula C₁₃H₁₃N₃O₃. The present invention describes additive/synergistic efficacy in the combination of a mosunetuzumab with lenalidomide, as well as potential overlapping toxicity, for which patients are carefully monitored and managed by the dosing regimens and treatment described herein.

### C. Additional Therapeutic Agents

In some instances, the methods described herein include administering mosunetuzumab and lenalidomide in combination with one or more additional therapeutic agents.

In some instances, the one or more additional therapeutic agents may reduce the rate or the severity of cytokine release syndrome (CRS). In some instances, the one or more additional therapeutic agents may prevent symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #50-42-8), or methylprednisolone (CAS #: 83-43-2)) or an IL-6R antagonist (e.g., tocilizumab, sarilumab, vobarilizumab (ALX-0061), satralizumab (SA-237), and variants thereof). In some instances, the additional therapeutic agent is tocilizumab. In some instances, the additional therapeutic agent is a corticosteroid. In some instances, a corticosteroid is administered prior to administration of mosunetuzumab. In some instances, the corticosteroid is administered 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of mosunetuzumab. In some instances, the corticosteroid is administered intravenously. In some instances, the corticosteroid is dexamethasone. In some instances, 10 mg of dexamethasone is administered to a subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of mosunetuzumab to the subject. In some instances, the corticosteroid is methylprednisolone. In some instances, the corticosteroid is prednisone.

In some instances, the one or more additional therapeutic agents may be used in the treatment of neutropenia. In some instances, the additional therapeutic agents may be prevent symptoms associated with neutropenia. In some instances, the additional therapeutic agents may reduce the rate or severity of neutropenia. In particular instances, the additional therapeutic agent is granulocyte colony-stimulating factor (G-CSF or GCSF) or colony-stimulating factor 3 (CSF 3). The mRNA sequence of human G-CSF/CSF 3 includes, e.g., NCBI RefSeq No. NM_000759, NM_001178147, NM_172219, and NM_172220, and the protein amino acid sequence of human G-CSF/CSF 3 includes, e.g., NCBI RefSeq No. NP_000750, NP_001171618, NP_757373, and NP_757374.

For all the methods described herein, mosunetuzumab and lenalidomide are formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. Mosunetuzumab and lenalidomide need not be, but are optionally formulated with, one or more agents currently used to prevent or treat the disorder in question. The effective amount of such other agents depends on the amount of mosunetuzumab and/or lenalidomide present in the formulation, the type of disorder or treatment, and other factors discussed above. Mosunetuzumab and lenalidomide may be suitably administered to the subject over a series of treatments. When mosunetuzumab and lenalidomide are administered on the same day, mosunetuzumab may be administered before, simultaneously with, or after administration of lenalidomide.

In some instances, additional therapeutic agents useful in the present invention include therapeutic antibodies, such as alemtuzumab (CAMPATH®), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (BEXXAR®, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, tafasitamab, talizumab, tefibazumab, tocilizumab, toralizumab, tucotuzumab celmoleukin, tucusituzumab, umavizumab, urtoxazumab, ustekinumab, visilizumab, and briakinumab.

### IV. Pharmaceutical Compositions and Formulations

Mosunetuzumab and/or lenalidomide described herein can be used in pharmaceutical compositions and formulations. Pharmaceutical compositions and formulations of mosunetuzumab, lenalidomide, and/or other agents describe herein (e.g., dexamethasone) can be prepared by mixing one, two, or all three agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Lenalidomide may also be formulated according to standard formulation and/or manufacturing practices. Dexamethasone may also be formulated according to standard formulation and/or manufacturing practices. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

In some embodiments, mosunetuzumab is formulated for administration intravenously. In some embodiments, lenalidomide is formulated for administration orally. In some embodiments, dexamethasone is formulated for administration intravenously.

### V. Kits and Articles of Manufacture

In another aspect of the invention, a kit or an article of manufacture containing materials useful for the treatment, prevention, and/or diagnosis of the disorders described above is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab or lenalidomide described herein. The label or package insert indicates that the composition is used for treating relapsed and/or refractory (R/R) follicular lymphoma (FL) and further includes information related to at least one of the dosing regimens described herein. In some embodiments, the label or package insert indicates that the composition is used for treating R/R FL in a patient who is R/R to at least one prior treatment (e.g., a chemo-immunotherapy) that comprised an anti-CD20 antibody (e.g., an anti-CD20 monoclonal antibody; e.g., rituximab or obinutuzumab). Moreover, the kit or article of manufacture may comprise (a) a first container with a composition contained therein, wherein the composition comprises mosunetuzumab, lenalidomide, or both mosunetuzumab and lenalidomide; and (b) a second container with a composition contained therein, wherein the composition comprises a further cytotoxic or otherwise therapeutic agent. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1. A Phase Ib, Open-Label, Non-Randomized, Multicenter Study Evaluating the Safety, Pharmacokinetics, and Efficacy of Mosunetuzumab in Combination with Lenalidomide in Patients with Relapsed or Refractory Follicular Lymphoma

### Study Design

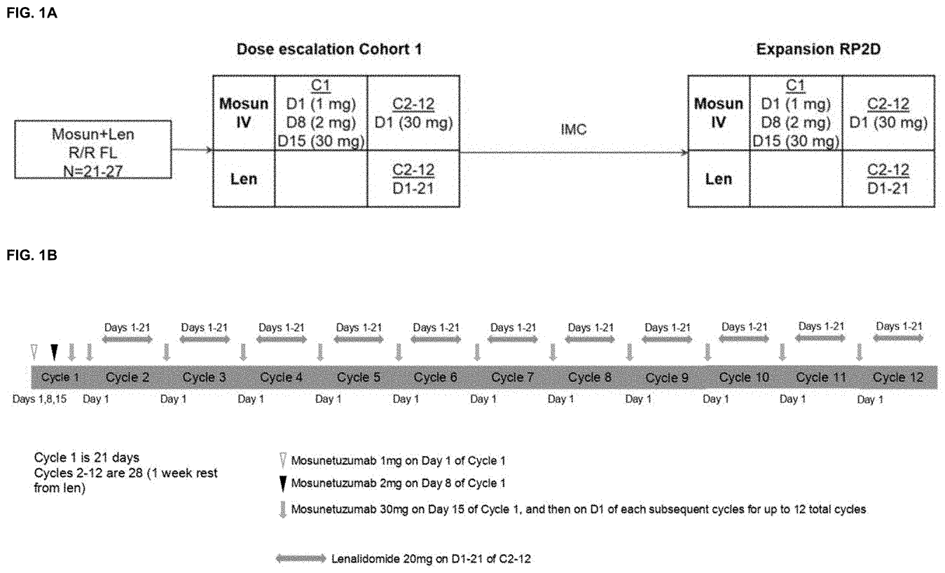

Study CO41942 is an ongoing Phase 1b, open-label, multicenter study evaluating the safety, pharmacokinetics, and efficacy of mosunetuzumab in combination with lenalidomide in patients with relapsed and/or refractory (e.g., R/R or r/r) follicular lymphoma (FL). Patients with r/r FL after at least one prior systemic therapy are eligible to enter the study. As of CCOD, mosunetuzumab in combination with lenalidomide is the only active treatment combination in Study CO41942, therefore this study focuses on the mosunetuzumab plus lenalidomide combination only. The study design is summarized in FIGS. 1A and 1B.

The study includes an initial dose-escalation phase, comprising a dose-escalation cohort, designed to determine the recommended Phase 2 dose (RP2D) of mosunetuzumab. The initial dose-escalation phase is followed by an expansion phase in which the RP2D for mosunetuzumab is given.

Study treatment is administered for 12 cycles; the duration of Cycle 1 is 21 days, and the duration of Cycle 2-12 is 28 days. Mosunetuzumab is administered intravenously: in Cycle 1 (21-day cycle), patients receive 1 mg on Day 1 (C1D1 dose); 2 mg on Day 8 (C1D2 dose); and 30 mg on Day 15 (C1D3 dose). In Cycles 2-12 (28-day cycles), patients receive 30 mg on Day 1 (C2D1-C12D1 doses). Lenalidomide is administered orally (PO) 20 mg once daily on Days 1-21 of Cycles 2-12 (28-day cycles). Lenalidomide is not administered on the last 7 days of each of the 28-day Cycles 2-12.

### Study Population and Treatment Received

As of CCOD (Apr. 5, 2021), all patients (N=16) were enrolled in the mosunetuzumab in combination with lenalidomide (IV) arm and received at least one dose of mosunetuzumab. Ten patients completed the DLT assessment window and reached D28 of Cycle 2. The 1/2/30 mg dose for mosunetuzumab was used for all patients. Lenalidomide 20 mg was the dose for all patients.

Overall, 9 patients (56.3%) had a duration on study of 0 to 3 months; 4 patients (25.0%) had a duration of 3 to 6 months; 3 patients (18.8%) had a duration of 6 to 9 months.

Patient demographics for the safety-evaluable population (N=16) are presented in Table 3. The majority of patients were female (62.5%; 10/16 patients) and had a median age of 61.5 years. Patient Eastern Cooperative Oncology Group (ECOG) status was 0 for 81.3% (13/16 patients) and 1 for 18.8% (3/16 patients).

TABLE 3
Demographic and baseline characteristics
of the safety-evaluable patients.

| | Patients treated |
|---|---|
| | with Mosunetuzumab |
| | 1/2/30 mg (N = 16) |
| Age (year) | |
| Mean (SD) | 60.8 (13.2) |
| Median | 61.5 |
| Min-Max | 31-79 |
| Sex | |
| Male | 6 (37.5%) |
| Female | 10 (62.5%) |
| Ethnicity | |
| Hispanic or Latino | 1 (6.3%) |
| Not Hispanic or Latino | 7 (43.8%) |
| Not stated | 8 (50%) |
| Race | |
| White | 8 (50%) |
| Unknown | 8 (50%) |
| Weight (kg) | |
| Mean | 78.68 (13.05) |
| Median | 75.15 |
| Min-Max | 58.3-100.5 |
| Height (cm) | |
| Mean | 167.78 (11.65) |
| Median | 165.50 |
| Min-Max | 153.0-190.0 |
| Body Mass Index (kg/m²) at baseline | |
| Mean | 27.92 (3.37) |
| Median | 29.12 |
| Min-Max | 20.8-33.1 |
| ECOG Status at Baseline | |
| 0 | 13 (81.3%) |
| 1 | 3 (18.8%) |

CCOD: Apr. 5, 2021

As of CCOD, all 16 patients in the safety-evaluable population had received at least one dose of study treatment. Sixteen patients entered Cycle 1, Day 1 (and received the C1D1 dose); 14 patients entered Cycle 1, Day 8 (and received the C1D2 dose) 14 patients entered Cycle 1, Day 15 (and received the C1D3 dose); 13 patients entered Cycle 2 (C2); and 8 patients entered Cycle 3 of treatment.

For mosunetuzumab, the median duration of treatment was 1.8 months (range: 0-7 months). The median number of treatment cycles was 3 (range: 1-9); the median number of doses received was 5 (range: 1-11); and the median cumulative dose received was 93 mg (range: 1.0-273.0).

For lenalidomide, the median duration of treatment was 1.9 months (range: 0-6 months). The median number of treatment cycles was 3 (range: 1-5); the median number of doses received was 37 (range: 1-151); and the median of cumulative dose received was 740 mg (range: 20.0-3020.0).

### Safety Results

The safety-evaluable population (N=16) included all patients who were enrolled in the study and received at least one dose of study treatment. An overview of key safety results obtained by the CCOD (Apr. 5, 2021) is summarized below and further described in Table 4.
The percentage of patients who reported at least one adverse event (AE) was 81.3% (13/16 patients), with a total of 83 AEs reported overall.
The most commonly reported AEs by PT were CRS; 31.3%; 5/16 patients); constipation, and neutropenia (both 25.0%; 4/16 patients).
The percentage of patients who reported at least one event considered related to study treatment was 81.3% (13/16 patients). The percentage of patients who reported at least one event considered related to mosunetuzumab or lenalidomide treatment was 81.3% and 50.0%, respectively.
As of CCOD, no fatal (Grade 5) AEs were reported in the study.
The percentage of patients who reported at least one serious adverse event (SAE) was 12.5% (2/16 patients), of which three events were related to mosunetuzumab treatment (by PT, CRS [2 events]; rash maculo-papular).
The percentage of patients who reported at least one Grade 3 AE was 31.3% (5/16 patients).
No AEs were reported that led to study treatment discontinuation.
The percentage of patients who reported at least one event that led to dose modification/interruption of study treatment was 37.5% (6/16 patients). Of these, 2 patients had dose interruption of mosunetuzumab and 6 patients had dose interruption of lenalidomide treatment.
The percentage of patients who reported at least one adverse event of special interest (AESI) was 31.3% (5/16 patients).

The incidence and frequency of AEs, including AEs considered related to mosunetuzumab, SAEs and AESIs were similar in this study as a related study with monotherapy mosunetuzumab administered with similar doses.

TABLE 4
Safety Summary for Safety-Evaluable
Patients as of CCOD of Apr. 5, 2021.

| | Mosunetuzumab + | Mosunetuzumab |
|---|---|---|
| | Lenalidomide (IV) | (IV) |
| | (N = 16) | (N = 213) |
| | N (%) | N (%) |
| Patients who experienced at | 13 (81.3) | 207 (97.2) |
| least one adverse event (AE) | | |
| Total number of AEs | 83 | 2258 |
| Deaths | 0 | 64 (30.0) |
| Fatal AEs^{a} | 0 | 5 (2.3) |
| Serious AEs | 2 (12.5) | 107 (50.2) |
| Grade 3-5 AEs | 5 (31.3) | 141 (66.2) |
| Treatment-related AEs | 13 (81.3) | 183 (85.9) |
| Mosunetuzumab | 13 (81.3) | 183 (85.9) |
| Lenalidomide | 8 (50.0) | — |
| AEs leading to treatment | 0 | 7 (3.3) |
| withdrawal | | |
| Mosunetuzumab | 0 | 7 (3.3) |
| Lenalidomide | 0 | — |
| AEs leading to dose | 6 (37.5) | 29 (13.6) |
| modification/interruption | | |
| Mosunetuzumab | 2 (12.5) | 29 (13.6) |
| Lenalidomide | 6 (37.5) | — |
| Patients who experienced at | | |
| least one AESI | | |
| Cytokine release syndrome^{b} | 5 (31.3) | 79 (37.1) |
| Neutropenia/neutrophil count | 4 (25.0) | 56 (26.3) |
| decrease | | |
| Febrile neutropenia | 0 | 6 (2.8) |
| Thrombocytopenia | 1 (6.3) | 14 (6.6) |
| Tumor lysis syndrome | 0 | 3 (1.4) |
| Tumor flare | 1 (6.3)^{c} | 4 (1.9) |
| AST increased | 1 (6.3) | 14 (6.6) |
| ALT increased | 1 (6.3) | 22 (10.3) |

AE = adverse event,
AESI = adverse event of special interest
^{a}Does not include events of “death due to progressive disease”
^{b}per ASTCT criteria
^{c}Subject had worsening pleural effusion. Tumor flare based on Medical Dictionary for Regulatory Activities (MedDRA) preferred terms (PT) of “tumor flare, tumor inflammation, tumor pain and new or worsening pleural effusion”.

Most Frequently Reported Adverse Events:

Overall, the percentage of patients who reported at least one AE was 81.3% (13/16 patients), with a total of 83 AEs reported overall.

The most commonly reported AEs by System Organ Class (SOC) were skin and subcutaneous tissue disorders (43.8%; 7/16 patients); gastrointestinal disorders (37.5%; 6/16 patients); blood and lymphatic system disorders; general disorders and administration site conditions, immune system disorders, and nervous system disorders (all 31.3%; 5/16 patients).

The most commonly reported AEs by MedDRA Preferred Term (PT) were CRS (31.3%; 5/16 patients); constipation and neutropenia (both 25.0%; 4/16 patients). A summary of AEs (≥10%) is presented in Table 5.

TABLE 5
Summary of Adverse Events with ≥10%
by patient, as of Apr. 5, 2021.

| MedDRA System Organ Class | Mosun + Len |
|---|---|
| MedDRA Preferred Term | (N = 16) |
| Skin and subcutaneous tissue disorders | |
| Dry skin | 3 (18.8%) |
| Pruritus | 3 (18.8%) |
| Erythema | 2 (12.5%) |
| Rash | 2 (12.5%) |
| Rash maculo-papular | 2 (12.5%) |
| Gastrointestinal disorders | |
| Constipation | 4 (25.0%) |
| Dry mouth | 2 (12.5%) |
| Blood and lymphatic system disorders | |
| Neutropenia | 4 (25.0%) |
| General disorders and administration site conditions | |
| Asthenia | 2 (12.5%) |
| Immune system disorders | |
| Cytokine release syndrome | 5 (31.3%) |
| Nervous system disorders | |
| headache | 2 (12.5%) |
| Musculoskeletal and connective tissue disorders | |
| Bone pain | 2 (12.5%) |
| Metabolism and nutrition disorders | |
| Decreased appetite | 2 (12.5%) |

Based on MedDRA version 23.1. Percentages are based on N in the column headings. Only treatment emergent AEs are displayed. For frequency counts by preferred term, multiple occurrences of the same AE in an individual are counted only once. CCOD of Apr. 5, 2021.

Adverse Events Related to Treatment

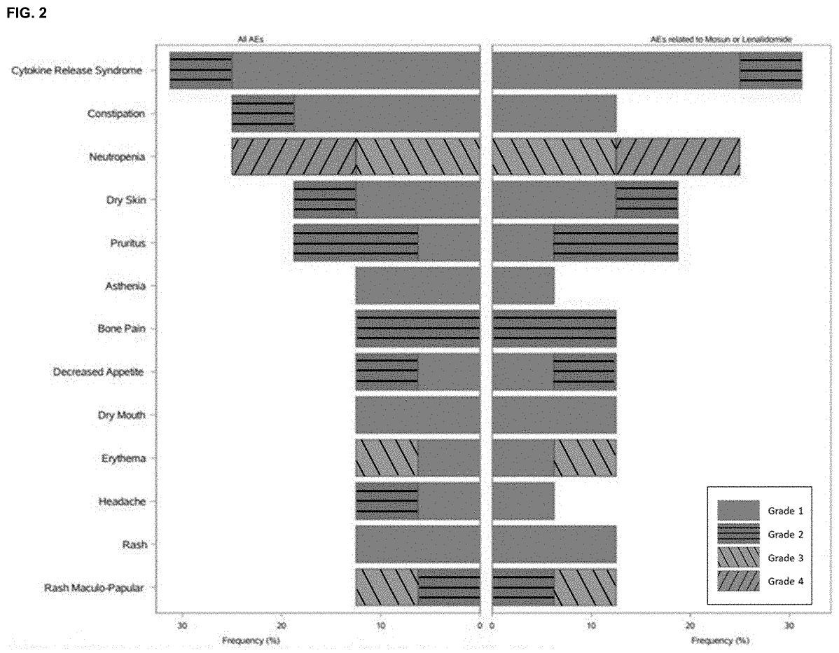

Overall, the percentage of patients who reported at least one event considered related to study treatment was 81.3% (13/16 patients; Table 4). The most commonly reported adverse events relating to treatment were CRS (31.3%; 5/16 patients) and neutropenia (25.0%; 4/16 patients). AEs (≥10%) considered related to study treatment (e.g., related to mosunetuzumab or lenalidomide) are shown in FIG. 2.

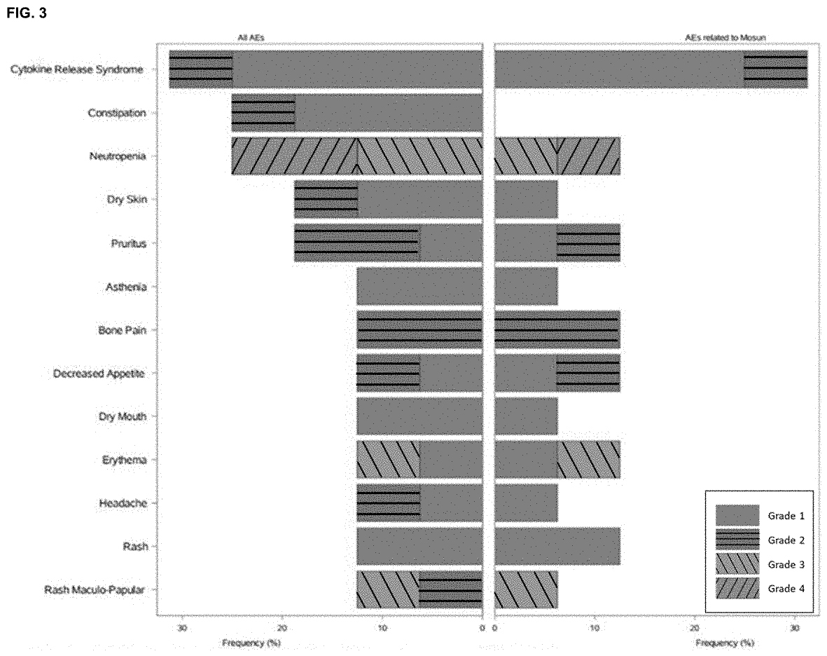

Overall, the percentage of patients who reported at least one AE considered related to mosunetuzumab treatment was 81.3% (13/16 patients; Table 4). The most commonly reported AE by PT considered related to mosunetuzumab treatment was CRS (31.3%; 5/16 patients). AEs (10%) considered related to mosunetuzumab are shown in FIG. 3.

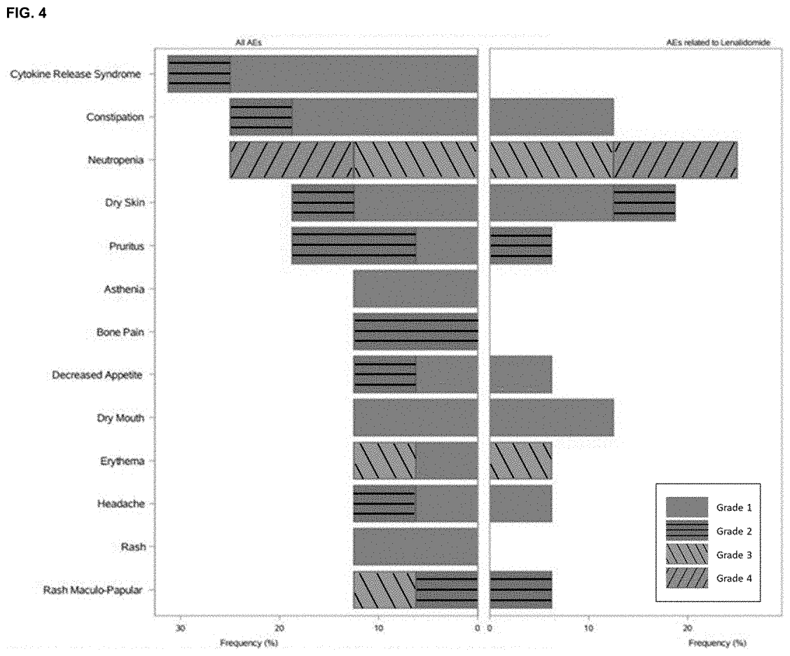

Overall, the percentage of patients who reported at least one AE considered related to lenalidomide treatment was 50.0% (8/16 patients; Table 4). The most commonly reported AE by PT considered related to lenalidomide treatment was neutropenia (25.0%; 4/16 patients). AEs (10%) considered related to lenalidomide are shown in FIG. 4.

Adverse Events by Intensity

Overall, the percentage of patients who reported at least one Grade ≥3 AE was 31.3% (5/16 patients; Table 4). The most commonly reported AE by PT was neutropenia (25.0%; 4/16 patients; Table 6).

TABLE 6
Summary of Adverse Events (AE) with
Grades 3-5, as of Apr. 5, 2021.

| | Patients |
|---|---|
| | treated with |
| | 1/2/30 mg |
| MedDRA System Organ Class | Mosun |
| MedDRA Preferred Term | (N = 16) |
| Total number of patients with at least one Grade 3-5 AE | 5 (31.3%) |
| Overall total number of events | 13 |
| Blood and lymphatic system disorders | |
| Total number of patients with at least one Grade 3-5 | 4 (25.0%) |
| AE | |
| Total number of events | 9 |
| Neutropenia | 4 (25.0%) |
| Skin and subcutaneous tissue disorders | |
| Total number of patients with at least one Grade 3-5 | 2 (12.5%) |
| AE | |
| Total number of events | 2 |
| Erythema | 1 (6.3%) |
| Rash maculo-papular | 1 (6.3%) |
| Investigations | |
| Total number of patients with at least one Grade 3-5 | 1 (6.3%) |
| AE | |
| Total number of events | 2 |
| Erythema | 1 (6.3%) |
| Rash maculo-papular | 1 (6.3%) |

Based on MedDRA version 23.1. Percentages are based on N in the column headings. Only treatment emergent AEs are displayed. For frequency counts of “Total number of events” rows, multiple occurrences of the same AE in an individual are counted separately.

Deaths

As of the CCOD, no deaths have been reported in Study CO41942.

Serious Adverse Events

Overall, the percentage of patients who reported at least one SAE was 12.5% (2/16 patients; Table 4). A summary of serious adverse events reported is provided in Table 7. One patient (6.3%) experienced an SAE (by PT, rash maculo-papular) considered related to mosunetuzumab by the Investigator. This event was of Grade 3 severity and resolved after 9 days, following a dose interruption of mosunetuzumab. Two other events (by PT, pleural effusion; atrial flutter) led to dose interruption of treatment (lenalidomide and mosunetuzumab, respectively); both events were Grade 2 severity and had resolved by Day 8 of onset. All other SAEs reported were of Grade 1 or Grade 2 severity, and resolved on or by Day 9 of onset without dose interruption/modification of study treatment. No SAEs of Grade 4 or 5 severity were reported.

TABLE 7
Summary of Serious Adverse Events (SAE), as of Apr. 5, 2021.

| | | Patients treated with |
|---|---|---|
| MedDRA System Organ Class | | 1/2/30 mg Mosun |
| MedDRA Preferred Term | Grade | (N = 16) |
| Any serious adverse events | Any Grade | 2 (12.5%) |
| | 1 | 0 |
| | 2 | 1 (6.3%) |
| | 3 | 1 (6.3%) |
| | 4 | 0 |
| | 5 | 0 |
| Cardiac disorders | | |
| Overall | Any Grade | 1 (6.3%) |
| | 1 | 0 |
| | 2 | 1 (6.3%) |
| | 3 | 0 |
| | 4 | 0 |
| | 5 | 0 |
| Atrial Flutter | Any Grade | 1 (6.3%) |
| | 1 | 0 |
| | 2 | 1 (6.3%) |
| | 3 | 0 |
| | 4 | 0 |
| | 5 | 0 |
| General disorders and | | |
| administration site conditions | | |
| Overall | Any Grade | 1 (6.3%) |
| | 1 | 0 |
| | 2 | 1 (6.3%) |
| | 3 | 0 |
| | 4 | 0 |
| | 5 | 0 |
| Adverse Drug Reaction | Any Grade | 1 (6.3%) |
| | 1 | 0 |
| | 2 | 1 (6.3%) |
| | 3 | 0 |
| | 4 | 0 |
| | 5 | 0 |
| Immune system disorders | | |
| Overall | Any Grade | 1 (6.3%) |
| | 1 | 0 |
| | 2 | 1 (6.3%) |
| | 3 | 0 |
| | 4 | 0 |
| | 5 | 0 |
| Cytokine release syndrome | Any Grade | 1 (6.3%) |
| | 1 | 0 |
| | 2 | 1 (6.3%) |
| | 3 | 0 |
| | 4 | 0 |
| | 5 | 0 |
| Respiratory, thoracic, and | | |
| mediastinal disorders | | |
| Overall | Any Grade | 1 (6.3%) |
| | 1 | 0 |
| | 2 | 1 (6.3%) |
| | 3 | 0 |
| | 4 | 0 |
| | 5 | 0 |
| Pleural effusion | Any Grade | 1 (6.3%) |
| | 1 | 0 |
| | 2 | 1 (6.3%) |
| | 3 | 0 |
| | 4 | 0 |
| | 5 | 0 |
| Skin and subcutaneous disorders | | |
| Overall | Any Grade | 1 (6.3%) |
| | 1 | 0 |
| | 2 | 0 |
| | 3 | 1 (6.3%) |
| | 4 | 0 |
| | 5 | 0 |
| Rash maculo-papular | Any Grade | 1 (6.3%) |
| | 1 | 0 |
| | 2 | 0 |
| | 3 | 1 (6.3%) |
| | 4 | 0 |
| | 5 | 0 |

Percentages are based on N in the column headings. For frequency counts by preferred term, multiple occurrences of the same SAE in an individual are counted only once. For frequency counts of “Total number of events” rows, multiple occurrences of the same SAE in an individual are counted separately. Adverse Events with missing Common Terminology Criteria for Adverse Events (CTCAE) grades are excluded.

Adverse Events that LED to Study Treatment Dose Modification or Interruption

Overall, the percentage of patients who reported at least one event that led to dose modification/interruption of study treatment was 37.5% (6/16 patients; Table 4). Of these, 2 patients had dose interruption of mosunetuzumab and 6 patients had dose interruption of lenalidomide treatment.

Two patients (12.5%) experienced events (by PT, rash maculo-papular [Grade 3]; neutropenia [Grade 4]; atrial flutter [Grade 2]) reported by the Investigator that led to dose interruption of mosunetuzumab. All events resolved within 9 days of onset following treatment for the AE.

Six patients (37.5%) experienced events (by PT, pleural effusion [Grade 2]; neutropenia, [Grade 3 and Grade 4, 3 events each]; erythema [Grade 3]; pruritus [Grade 2]; rash maculo-papular [Grade 3]) that led to dose interruption of lenalidomide. Two patients (12.5%) experienced an event (by PT, neutropenia [Grade 3 and Grade 4]) that led to dose reduction of lenalidomide. All events resolved, except for two events of neutropenia (both Grade 3 severity), which were still ongoing as of the CCOD.

Adverse Events of Special Interest (AESI)

AESIs include events that are considered to be of overlapping toxicities between the study treatments, and are summarized in Table 8. Overall, the percentage of patients who reported at least one AESI was 43.8%(7/16 patients). The most commonly reported AESIs were CRS (31.3%; 5/16 patients), and neutropenia (25.0%; 4/16 patients). One patient (6.3%) reported an event of Grade 1 thrombocytopenia that had not resolved at the time of CCOD; no intervention was taken.

TABLE 8
Summary of Adverse Events of Special
Interest (AESI), as of Apr. 5, 2021.

| | Patients |
|---|---|
| | treated with |
| | 1/2/30 mg |
| MedDRA System Organ Class | Mosun |
| MedDRA Preferred Term | (N = 16) |
| Total number of patients with at least one AESI | 7 (43.8) |
| Overall total number of events | 20 |
| Immune system disorders | |
| Total number of patients with at least one AESI | 5 (31.3%) |
| Total number of events | 7 |
| Cytokine release syndrome | 5 (31.3%) |
| Blood and lymphatic system disorders | |
| Total number of patients with at least one AESI | 4 (25.0%) |
| Total number of events | 10 |
| Neutropenia | 4 (25.0%) |
| Thrombocytopenia | 1 (6.3%) |
| Investigations | |
| Total number of patients with at least one AESI | 1 (6.3%) |
| Total number of events | 2 |
| Alanine aminotransferase increased | 1 (6.3%) |
| Aspartate aminotransferase increased | 1 (6.3%) |
| Respiratory, thoracic, and mediastinal disorders | |
| Total number of patients with at least one AESI | 1 (6.3%) |
| Total number of events | 1 |
| Pleural effusion | 1 (6.3%) |

Percentages are based on N in the column headings.

Overall, the most commonly reported AESI was CR (31.3%; 5/16 patients; 7 events). Of the patients who experienced an event, 4 patients (25.0%) reported an event of Grade 1 severity, and one patient (6.3%) reported an event of Grade 2 severity; all events were considered related to mosunetuzumab treatment. The majority of events were reported in patients on study day 1, all of which resolved within 3 days of onset. Two patients (12.5%) reported subsequent events: one patient on study day 15 and another on study day 29, which resolved within 2 and 7 days, respectively. Of the 5 patients who reported CRS, 4 patients (25.0%) received interventional treatment for this event.

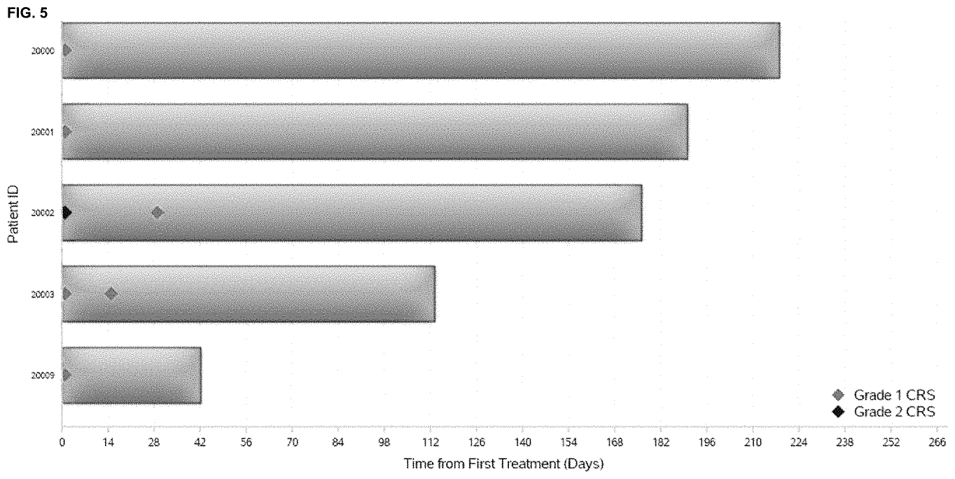

Five patients (31.3%) had a CRS event, considered related to mosunetuzumab treatment, as assessed by the Investigator, including 4 patients (25.0%) with ASTCT Grade 1, and one patient (6.3%) with Grade 2 (Table 9). All 5 patients had onset of first CRS event between Day 1 of Cycle 1 and Day 7 of Cycle 1 (FIG. 5). One patient (6.3%; 1/14) had a subsequent Grade 1 event between Day 15 of Cycle 1 and Day 21 of Cycle 1 during mosunetuzumab monotherapy treatment, and another patient (6.3%; 1/14) had a subsequent Grade 1 event in C2 during mosunetuzumab and lenalidomide combination treatment.

TABLE 9
CRS Events by Dose Cycle for Safety-Evaluable
Patients as of CCOD of Apr. 5, 2021.

| | Cycle 1 | Cycle 1 | Cycle 1 | | |
|---|---|---|---|---|---|
| | Days 1-7 | Days 8-14 | Days 15-21 | Cycle 2 | Cycle 3 |
| CRS Grade | N = 16 | N = 14 | N = 14 | N = 13 | N = 8 |
| Any Grade | 5 (31.3%) | 0 | 1 (7.1%) | 1 (7.1%) | 0 |
| 1 | 4 (25.0%) | 0 | 1 (7.1%) | 1 (7.1%) | 0 |
| 2 | 1 (6.3%) | 0 | 0 | 0 | 0 |
| 3 | 0 | 0 | 0 | 0 | 0 |
| 4 | 0 | 0 | 0 | 0 | 0 |
| 5 | 0 | 0 | 0 | 0 | 0 |

Overall, fever was the most frequent symptom of CRS, of which 7 events were reported in 5 patients who reported an event of CRS. Other signs and symptoms reported concurrently CRS included chills, hypoxia, tachycardia, elevated liver enzyme (AST), elevated liver enzyme (ALT), hyperuricemia and lactate dehydrogenase (LDH) increased. One patient (6.3%) received low-flow oxygen for the treatment of Grade 2 CRS. No Grade ≥3 CRS events were observed and no patient required tocilizumab, ICU management, high flow oxygen or vasopressor support.

Four patients (25.0%) reported 9 events of neutropenia (Table 4). Of these, 2 patients experienced 5 events considered related to mosunetuzumab and lenalidomide treatment, and 2 patients experienced 4 events considered related to lenalidomide treatment only, as assessed by the Investigator. Two patients reported events of Grade 4 severity, all of which resolved, one patient without any treatment and one patient following treatment with G-CSF. Two patients reported 5 events of Grade 3 severity, of which 2 events (one in each patient) did not resolve as of the CCOD, following treatment with G-CSF for the event and dose interruption of lenalidomide. Events of neutropenia occurred between Day 42 and Day 155, and had a duration of between 8 and 16 days.

### Preliminary Efficacy

The efficacy evaluable population included all patients who had been assessed for response at any time on study, who had withdrawn from treatment or study prior to reaching their first response assessment, or who had been on study long enough to have reached their first scheduled response assessment, planned per protocol for Cycle 3, D15-21. The response was assessed by the Investigator per Lugano 2014 criteria (Cheson B D, et al. J Clin Oncol 2014; 32:1-9).

No formal efficacy data analysis was performed. Upon review of the electronic database, as of CCOD, 8 patients enrolled in Study CO41942 had at least one response assessment. Of these 8 patients, 2 patients had a partial response, and 6 patients had a complete response (i.e., an overall response rate (OR) of 100% and a complete response rate (CR) of 75%. No patients had progression of the disease or withdrew from the study.

### Pharmacokinetics

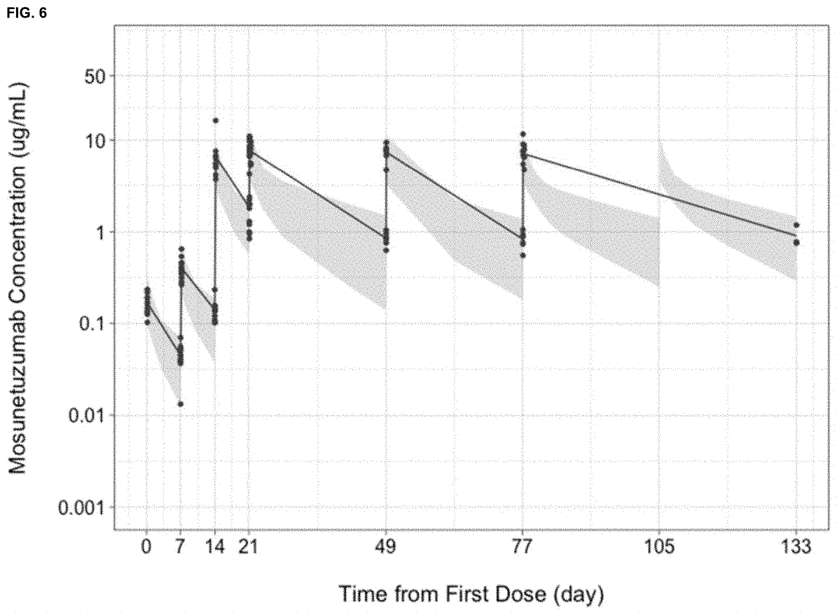

Preliminary mosunetuzumab pharmacokinetic data (N=13) in patients who received the 1/2/30 mg Q4W dose of mosunetuzumab in Study CO41942 are summarized in FIG. 6. Observed pharmacokinetics of mosunetuzumab at 1/2/30 mg (IV) in combination with lenalidomide in R/R FL patients were similar to predictions of mosunetuzumab pharmacokinetics of mosunetuzumab monotherapy in R/R NHL patients at the same dose. Preliminary results indicate that mosunetuzumab PK is similar when administered in combination with lenalidomide as compared to monotherapy, where the observed concentration measurements fell generally within the popPK model simulated 5th to 95^{th} prediction intervals.

No lenalidomide PK data were available at the time of this report. Mosunetuzumab is not expected to impact the PK of lenalidomide which is primarily excreted in the urine (Chen N et al. Clin Pharmacokinet. 2017; 56(2):139-152).

### Conclusion

The safety profile for mosunetuzumab, in combination with lenalidomide, at the tested dose and schedule (1/2/30 mg, Q4W) was acceptable, with no unexpected safety signals.

The overall frequency and severity of CRS events (7 events of Grade 1-2 severity, in 5 patients) observed with mosunetuzumab in combination with lenalidomide in this study was comparable to CRS events observed with mosunetuzumab monotherapy. All CRS events were Grade 1, except in one patient who experienced a Grade 2 event in Cycle 1 with mosunetuzumab (1 mg) monotherapy. This patient was the only patient who experienced a CRS event in Cycle 2, which was Grade 1. Four patients (25.0%) reported events of Grade 3-4 neutropenia. Of the 9 events reported, 7 events resolved and 2 events (Grade 3 severity) had not resolved as of CCOD. One patient (6.3%) reported an event of Grade 1 thrombocytopenia that had not resolved at the time of CCOD. No patients discontinued study treatment due to AEs.

Mosunetuzumab PK exposures observed in Study CO41942 were consistent with what was expected based on PK observed with mosunetuzumab monotherapy, suggesting that the combination of mosunetuzumab with lenalidomide does not appear to affect mosunetuzumab exposure. The proposed dosing regimen was supported by the totality of data and the exposure-response characterizations based on mosunetuzumab monotherapy and mosunetuzumab in combination with lenalidomide, suggesting an adequate balance of clinical benefit/risk in the patient population.

In summary, the available data from Study GO41942 demonstrated an acceptable safety profile and preliminary efficacy for patients with R/R FL, thus supporting the continued use of 1/2/30 mg step-up dosing regimen of mosunetuzumab in combination with lenalidomide.

### Example 2. Mosunetuzumab in Combination with Lenalidomide has a Manageable Safety Profile and Encouraging Activity in Patients with Relapsed/Refractory Follicular Lymphoma: Initial Results from a Phase Ib Study

Presented here are initial data from an ongoing Phase Ib study (NCT04246086) evaluating the safety and activity of combination therapy of mosunetuzumab and lenalidomide in R/R FL patients who have received at least one prior line of therapy.

### Methods

Patients with R/R FL (Grade 1-3a) and at least one prior systemic anti-cancer therapy were enrolled to receive 12 cycles of combination therapy of mosunetuzumab and lenalidomide (cycle duration: Cycle 1, 21 days; Cycles 2-12, 28 days). In Cycle 1, step-up doses of mosunetuzumab (IV infusion) were given on Day 1 of Cycle 1 (the C1D1 dose; 1 mg) and Day 2 of Cycle 1 (the C1D2 dose; 2 mg), with the target dose given on Day 3 of Cycle 1 (the C1D3 dose; 30 mg) and on Day 1 of Cycles 2-12 (the C2D1-C12D1 doses). Lenalidomide (20 mg orally) was administered on Days 1-21 of Cycles 2-12. No hospitalization was mandated by the protocol. The primary objective was to evaluate the safety of combination therapy of mosunetuzumab and lenalidomide; secondary objectives include assessment of response and long-term efficacy outcomes. Cytokine release syndrome (CRS) was reported using ASTCT criteria (Lee et al. Biol Blood Marrow Transplant 2019). Responses are assessed by investigators with PET-CT using Lugano 2014 criteria (Cheson B D, et al. J Clin Oncol 2014; 32:1-9).

### Results

At data cut-off (May 31, 2021), 27 patients had been enrolled. Median age was 59 years (range: 31-79 years); 12 patients (44%) were male. All patients had an ECOG (Eastern Cooperative Oncology Group) performance status of 0 (18 patients, 67%) or 1 (9 patients, 33%). Median number of prior lines of therapy was 1 (range: 1-4), and 3 patients (11%) had disease progression (PD)<24 months from the start of first-line therapy. At data cut-off, 16 patients (59%) had been on study for 0-3 months, 8 (30%) for 3-6 months, 2 (7%) for 6-9 months, and 1 (4%) for >9 months.

All 27 patients were safety evaluable at data cut-off. Twenty patients (74%) experienced 1 adverse event (AE) of any Grade (Gr); the most common AE was CRS (8 patients, 30%). Grade 3-4 AEs occurred in 8 patients (30%) and serious AEs in 8 patients (30%). No Grade 5 (fatal) AEs were observed. AEs relating to mosunetuzumab occurred in 20 patients (74%) and AEs relating to lenalidomide occurred in 10 patients (37%). There were no AEs leading to withdrawal of mosunetuzumab or lenalidomide; 2 patients had mosunetuzumab-related AEs leading to mosunetuzumab dose delays, 6 patients (22%) had lenalidomide-related AEs leading to lenalidomide dose interruption and/or reduction.

In all patients, CRS events were Grade 1 (7/8 patients) or Grade 2 (1/8 patients). For most patients (6/8), CRS events occurred in Cycle 1; 2 patients experienced Grade 1 CRS events in Cycle 2. Median time to CRS onset was 1 day after first study drug administration (range: 1-28 days), median CRS duration was 3 days (range: 2-5 days). All CRS events resolved without sequelae. No patient required tocilizumab, ICU admission, high flow oxygen, or vasopressor support. Five patients (19%) reported 14 events of Grade 3-4 neutropenia; all neutropenia events occurred between Day 41 and Day 218, with a duration of 6-16 days. All neutropenia events resolved, with 1 patient receiving primary granulocyte colony-stimulating factor (G-CSF) prophylaxis and 2 patients receiving G-CSF treatment. No febrile neutropenia events occurred.

The efficacy-evaluable population included all patients who had been assessed for response at any time on study, who had withdrawn from treatment or study prior to reaching their first response assessment, or who had been on study long enough to have reached their first scheduled response assessment, planned per protocol for Days 15-21 of Cycle 3. Objective response rate in the 13 patients who were efficacy evaluable at data cut-off was 92%, with complete metabolic response observed in 10 patients (77%), partial metabolic response (PMR) in 2 patients (15%), and stable disease in 1 patient (8%). One patient who initially achieved PMR experienced PD after 8 cycles of treatment.

### Conclusion

Combination treatment of mosunetuzumab and lenalidomide appeared to have an acceptable safety profile in patients with R/R FL who had received at least one prior line of therapy, with encouraging preliminary anti-lymphoma activity observed.

### Example 3. Preliminary Safety and Efficacy for Mosunetuzumab in Combination with Lenalidomide in Patients with Relapsed/Refractory Follicular Lymphoma: Initial Results from a Phase Ib Study

Presented here are additional data from an ongoing Phase Ib study (NCT04246086) (described in Example 2 above) evaluating the safety and activity of combination therapy of mosunetuzumab and lenalidomide in R/R FL patients who have received at least one prior line of therapy.

### Methods

Patients with R/R FL (Grade 1-3a) and at least one prior systemic anti-cancer therapy were enrolled to receive 12 dosing cycles of combination therapy of mosunetuzumab and lenalidomide (cycle duration: Cycle 1, 21 days; Cycles 2-12, 28 days). In Cycle 1, step-up doses of mosunetuzumab (IV infusion) were given on Day 1 of Cycle 1 (the C1D1 dose; 1 mg) and Day 2 of Cycle 1 (the C1D2 dose; 2 mg), with the target dose given on Day 3 of Cycle 1 (the C1D3 dose; 30 mg) and on Day 1 of Cycles 2-12 (the C2D1-C12D1 doses). Lenalidomide (20 mg orally) was administered on Days 1-21 of Cycles 2-12. Corticosteroid pre-medication was administered to the patients during Cycles 1 and 2 and was optional from Cycle 3 onwards. For corticosteroid premedication, 10 mg of dexamethasone was administered intravenously to the patients prior to each dose of mosunetuzumab. Corticosteroid pre-medication may additionally be deemed optional on Cycle 2 depending on further safety data evaluation. No hospitalization was mandated by the protocol. The primary objective was to evaluate the safety of combination therapy of mosunetuzumab and lenalidomide; secondary objectives include assessment of response and long-term efficacy outcomes. Cytokine release syndrome (CRS) was reported using ASTCT criteria (Lee et al. Biol Blood Marrow Transplant 2019). Responses are assessed by investigators with PET-CT using Lugano 2014 criteria (Cheson B D, et al. J Clin Oncol 2014; 32:1-9).

### Results

At data cut-off (Sep. 13, 2021), 29 patients had been enrolled. Median age was 59 years (range: 30-79 years); 13 patients (44.8%) were male. All patients had an ECOG (Eastern Cooperative Oncology Group) performance status of 0-2. Two (6.8%) patients were Ann Arbor stage 1-11 at study entry and 27 (93.1%) patients were Ann Arbor stage III-IV at study entry (see Carbone et al., Cancer Res. 1971, 31(11): 1860-1861). Seven (24.1%) patients had 0-1 FLIPI risk factors at study entry, eight (27.6%) patients had 2 FLIPI risk factors at study entry, and 14 (48.3%) patients had 3-5 FLIPI risk factors at study entry. 18 (62.1%) patients had FL Grade 1-2, 5 (17.2%) patients had FL Grade 3a, and six (20.7%) patients had FL of an unknown Grade. Median number of prior lines of therapy was 1 (range: 1-6), and 3 patients (10.3%) had disease progression (PD)<24 months from the start of first-line therapy (POD24). Nine (31%) patients were refractory to at least one prior therapy comprising an anti-CD20 monoclonal antibody. Seven (24.1%) patients were double refractory to both a therapy comprising an anti-CD20 monoclonal antibody and a therapy comprising an alkylating agent. At data cut-off, 1 patient (3.4%) had been on study for 0-3 months, 14 (48.3%) for 3-6 months, 7 (24.1%) for 6-9 months, and 7 (24.1%) for >9 months. Median duration of mosunetuzumab treatment was 4.9 months (range 2-11) and median duration of lenalidomide treatment was 4.3 months (range 2-10). Median duration of follow-up was 5.4 months (range 3-12).

All 29 patients were safety evaluable at data cut-off (Sep. 13, 2021). Adverse events are summarized below in Table 10.

TABLE 10
Adverse Events in Safety Evaluable Patients
(data cut-off: Sep. 13, 2021)

| | N = 29 (%) |
|---|---|
| Any AE | 29 (100) |
| Related to mosunetuzumab/lenalidomide | 27 (93.1)/23 (79.3) |
| Grade 3-4 AE | 13 (44.8) |
| Related to mosunetuzumab/lenalidomide | 1 (3.4)/1 (3.4) |
| Serious AE | 9 (31.0) |
| Related to mosunetuzumab/lenalidomide | 6 (20.7)/1 (3.4) |
| Grade 5 (fatal) AE | 0 |
| AE leading to mosunetuzumab/lenalidomide | 0/1 (3.4) |
| withdrawal | |
| AE leading to mosunetuzumab/lenalidomide | 6 (20.7)/12 (41.4) |
| dose modification/interruption | |

AE = adverse event

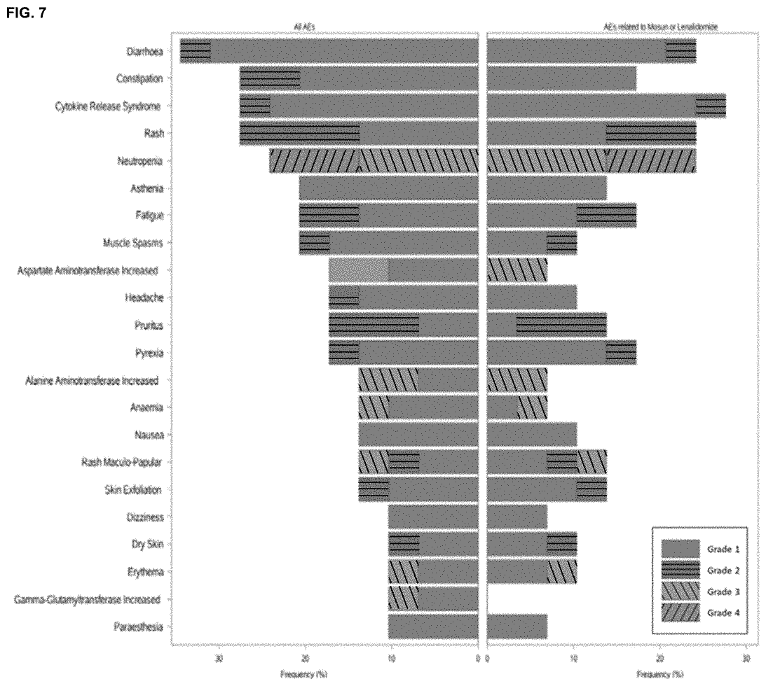

Twenty-nine patients (100%) experienced 1 adverse event (AE) of any Grade. Common adverse events, experienced by at least 10% of patients, are summarized in FIG. 7. Grade 3-4 AEs occurred in 13 patients (44.8%) and serious AEs in 9 patients (31%). No Grade 5 (fatal) AEs were observed. AEs relating to mosunetuzumab occurred in 27 patients (93.2%) and AEs relating to lenalidomide occurred in 23 patients (79.3%%). There were no AEs leading to withdrawal of mosunetuzumab and one AE (3.4%) leading to withdrawal of lenalidomide; 6 patients (20.7%) had mosunetuzumab-related AEs leading to mosunetuzumab dose modification and/or interruption; 12 patients (41.4%) had lenalidomide-related AEs leading to lenalidomide dose modification and/or interruption.

Cytokine release syndrome (CRS) events observed in the safety-evaluable patients are summarized below in Table 11.

TABLE 11
CRS Events in Safety Evaluable Patients
(data cut-off: Sep. 13, 2021)

| | N = 29 (%) |
|---|---|
| Any CRS (all Grades 1-2) | 8 (27.6) |
| Grade 1 | 7 (24.1) |
| Grade 2^{a} | 1 (3.4) |
| Median time to CRS onset, days (range) | 1 (1-28) |
| Median duration of CRS, days (range) | 3 (2-5) |

^{a}patient had fever and hypoxia that required 2 L nasal cannula oxygen

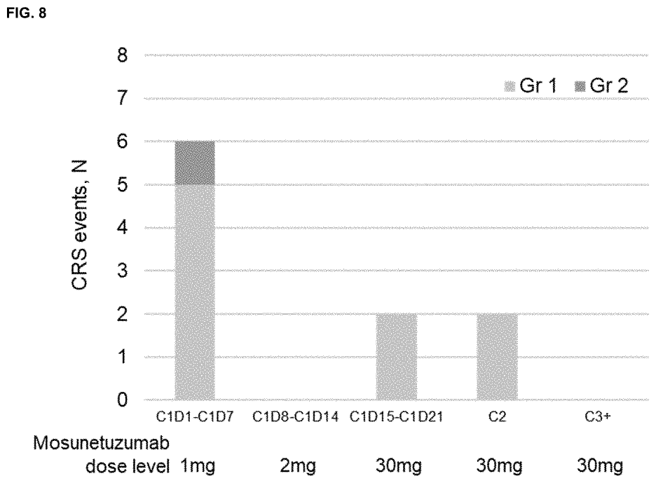

In all patients who experienced at least one CRS event (8/29 total patients), CRS events were Grade 1 (7/8 patients) or Grade 2 (1/8 patients). For most patients (6/8), CRS events occurred in Cycle 1; 2 patients experienced Grade 1 CRS events in Cycle 2. Median time to CRS onset was 1 day after first study drug administration (range: 1-28 days), median CRS duration was 3 days (range: 2-5 days). All CRS events resolved by data cut-off date (Sep. 13, 2021). No patient received tocilizumab, and no patient required ICU admission, high flow oxygen, or vasopressor support. No immune effector cell-associated neurotoxicity syndrome (ICANS) events occurred. A summary of CRS events by dosing cycle and dose is shown in FIG. 8.

Neutropenia and febrile neutropenia events observed in the safety-evaluable patients are summarized below in Table 12.

TABLE 12
Neutropenia and Febrile Neutropenia Events in Safety
Evaluable Patients (data cut-off: Sep. 13, 2021)

| | N = 29 (%) |
|---|---|
| Any neutropenia (all Grade 3-4) | 7 (24.1) |
| Grade 3 | 4 (13.4) |
| Grade 4 | 3 (10.3) |
| Median time to neutropenia onset, days (range) | 86 (41-189) |
| Median duration of neutropenia, days (range) | 9 (4-29) |

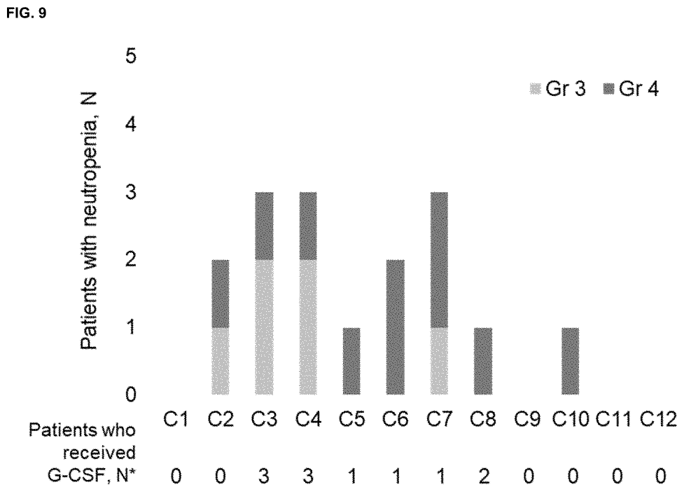

Seven patients (24.1%) reported neutropenia or febrile neutropenia events of Grade 3-4; all neutropenia events occurred between Day 41 and Day 189 (median 86 days), with a median duration of 9 days (range 4-29). All neutropenia events resolved by data cut-off date. Febrile neutropenia (Grade 3) occurred in 1 patient (duration: 5 days) and was considered treatment-related. A summary of neutropenia events reported by dosing cycle and Grade and granulocyte colony-stimulating factor (G-CSF) use by dosing cycle is shown in FIG. 9.

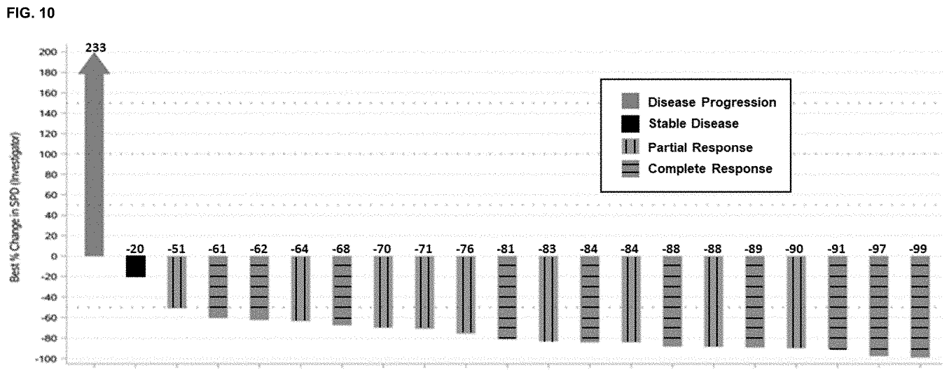

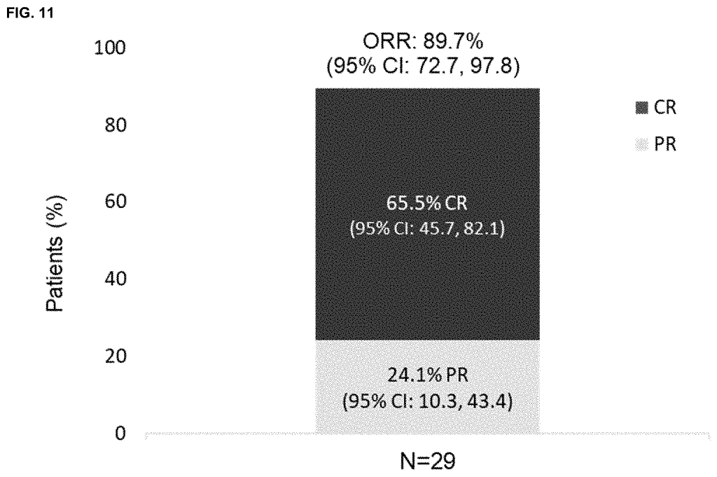

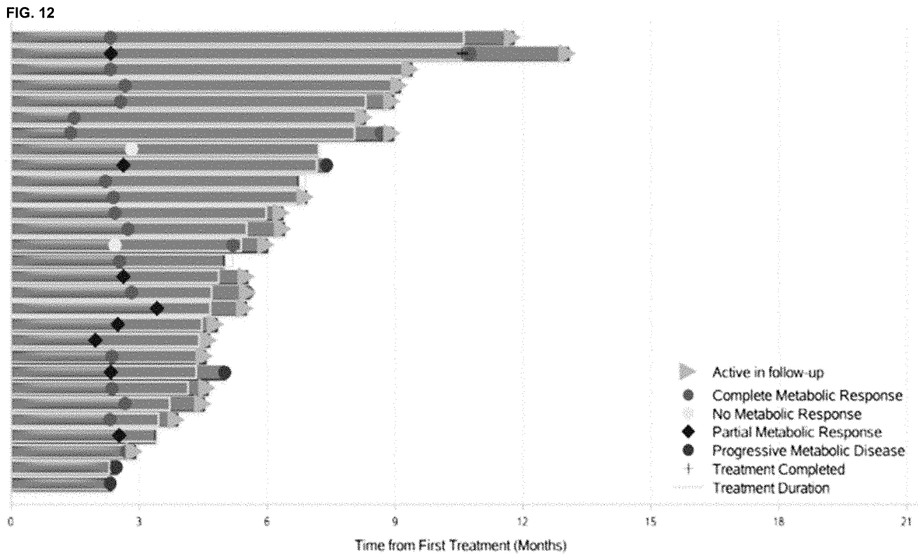

Anti-tumor activity in evaluable patients is shown in FIG. 10. In particular, objective response rate in the 29 patients at data cut-off was 89.7%, with complete metabolic response observed in 65.5% of patients (see FIG. 11). Notably, 3/3 patients with history of POD24, 9/9 patients with anti-CD20 refractory disease, and 7/7 patients with double-refractory disease all exhibited responses as measured by PET-CT. Duration of response in all 29 patients as of the data cut-off date of Sep. 13, 2021, is summarized in FIG. 12.

### Conclusion

Combination treatment of mosunetuzumab and lenalidomide appeared to have an acceptable safety profile in patients with R/R FL who had received at least one prior line of therapy, with encouraging anti-lymphoma activity and anti-tumor activity observed. No increase in CRS and neutropenia events was observed compared with mosunetuzumab monotherapy.

### EMBODIMENTS

Some embodiments of the technology described herein can be defined according to any of the following numbered embodiments:

1. A method of treating a subject, comprising administering to the subject an effective amount of mosunetuzumab and an effective amount of lenalidomide, wherein the subject:
(a) has relapsed or refractory follicular lymphoma (R/R FL) that expresses CD20, and
(b) has been previously treated with at least one chemo-immunotherapy regimen.

2. Mosunetuzumab for use in combination with lenalidomide in treating a subject, wherein the subject:
(a) has relapsed or refractory follicular lymphoma (R/R FL) that expresses CD20, and
(b) has been previously treated with at least one chemo-immunotherapy regimen.

3. Use of mosunetuzumab in combination with lenalidomide in treating a subject, wherein the subject:
(a) has relapsed or refractory follicular lymphoma (R/R FL) that expresses CD20, and
(b) has been previously treated with at least one chemo-immunotherapy regimen.

4. Use of mosunetuzumab in the manufacture of a medicament for use in combination with lenalidomide for treating a subject, wherein the subject:
(a) has relapsed or refractory follicular lymphoma (R/R FL) that expresses CD20, and
(b) has been previously treated with at least one chemo-immunotherapy regimen.

5. Use of lenalidomide in the manufacture of a medicament for use in combination with mosunetuzumab for treating a subject, wherein the subject:
(a) has relapsed or refractory follicular lymphoma (R/R FL) that expresses CD20, and
(b) has been previously treated with at least one chemo-immunotherapy regimen.

6. Use of mosunetuzumab and lenalidomide in the manufacture of a medicament for treating a subject, wherein the subject:
(a) has relapsed or refractory follicular lymphoma (R/R FL) that expresses CD20, and
(b) has been previously treated with at least one chemo-immunotherapy regimen.

7. The method, mosunetuzumab for use, or use of any one of embodiments 1-6, wherein at least one chemo-immunotherapy regimen comprises an anti-CD20 monoclonal antibody.

8. The method, mosunetuzumab for use, or use of any one of embodiments 1-7, wherein the subject has received only one prior line of systemic therapy and either:
(a) has a Follicular Lymphoma International Prognostic Index (FLIPI; Solal-Céligny et al. Blood. 2004; 104 (5): 1258-1265.) score of 2-5,
(b) has been refractory to prior anti-CD20 monoclonal antibody treatment, or
(c) has disease progression within 24 months after initiation of prior therapy.

9. The method of any one of embodiment 7 or 8, wherein the subject has not been treated with the anti-CD20 monoclonal antibody for at least 4 weeks prior to being administered the effective amount of mosunetuzumab and lenalidomide.

10. The mosunetuzumab for use or use of embodiment 7 or 8, wherein mosunetuzumab and lenalidomide are not to be administered to the subject for at least 4 weeks after the subject has received treatment with an anti-CD20 monoclonal antibody.

11. The method, mosunetuzumab for use, or use of any one of embodiments 1-6, wherein mosunetuzumab and lenalidomide have a synergistic effect on the R/R FL.

12. The method, mosunetuzumab for use, or use of embodiment 11, wherein the synergistic effect is a partial response, as defined by Lugano 2014 criteria (Cheson B D, et al. J Clin Oncol 2014; 32:1-9).

13. The method, mosunetuzumab for use, or use of embodiment 11, wherein the synergistic effect is a complete response, as defined by Lugano 2014 criteria (Cheson B D, et al. J Clin Oncol 2014; 32:1-9).

14. The method of any one of embodiments 1 and 7-9, and 11-13, wherein administering the effective amount of mosunetuzumab comprises administering mosunetuzumab according to a dosing regimen comprising at least a first dosing cycle and a second dosing cycle, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second dosing cycle comprises a single dose (C2D1) of mosunetuzumab, wherein the C2D1 is equal to or greater than the C1D3 and is between 0.2 mg to 50 mg.

15. The mosunetuzumab for use or use of any one of embodiments 2-8 and 10-14, wherein the mosunetuzumab is to be administered to the subject according to a dosing regimen comprising at least a first dosing cycle and a second dosing cycle, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second dosing cycle comprises a single dose (C2D1) of mosunetuzumab, wherein the C2D1 is equal to or greater than the C1D3 and is between 0.2 mg to 50 mg.

16. The method, mosunetuzumab for use, or use of embodiment 14 or 15, wherein:
(a) the C1D1 is between 0.4 mg to 4.0 mg, the C1D2 is between 1.0 mg to 20.0 mg, and the C1D3 is between 3.0 mg to 50.0 mg; and
(b) the C2D1 is between 3.0 mg to 50.0 mg.

17. The method, mosunetuzumab for use, or use of any one of embodiments 14-16, wherein:
(a) the C1D1 is between 0.8 mg to 3.0 mg, the C1D2 is between 1.0 mg to 6.0 mg, and the C1D3 is between 3.0 mg to 45.0 mg; and
(b) the C2D1 is between 3.0 mg to 45.0 mg.

18. The method, mosunetuzumab for use, or use of any one of embodiments 14-17, wherein the C1D1 and C1D2 are each less than the C1D3.

19. The method, mosunetuzumab for use, or use of any one of embodiments 14-17, wherein the C1D2 is greater than the C1D1 by about 50% to about 250%.

20. The method, mosunetuzumab for use, or use of any one of embodiments 14-17, wherein:
(a) the C1D1 is 0.8 mg, the C1D2 is 2.0 mg, and the C1D3 is 4.2 mg, and the C2D1 is 4.2 mg;
(b) the C1D1 is 1.0 mg, the C1D2 is 1.0 mg, and the C1D3 is 3.0 mg, and the C2D1 is 30.0 mg; or
(c) the C1D1 is 1.0 mg, the C1D2 is 2.0 mg, and the C1D3 is 30.0 mg, and the C2D1 is 30.0 mg.

21. The method, mosunetuzumab for use, or use of any one of embodiments 14-20, wherein the length of the first dosing cycle is 21 days.

22. The method of embodiment 21, wherein the method comprises administering to the subject the C1D1, the C1D2, and the C1D3 on or about Days 1, 8, and 15, respectively, of the first dosing cycle.

23. The mosunetuzumab for use or use of embodiment 21, wherein the C1D1, the C1D2, and the C1D3 are to be administered to the subject on or about Days 1, 8, and 15, respectively, of the first dosing cycle.

24. The method, mosunetuzumab for use, or use of any one of embodiments 14-23, wherein the length of the second dosing cycle is 28 days.

25. The method of embodiment 24, wherein the method comprises administering to the subject the C2D1 on Day 1 of the second dosing cycle.

26. The mosunetuzumab for use or use of embodiment 24, wherein the C2D1 is to be administered to the subject on Day 1 of the second dosing cycle.

27. The method, mosunetuzumab for use, or use of any one of embodiments 24-26, wherein the dosing regimen comprises one or more additional dosing cycles.

28. The method, mosunetuzumab for use, or use of embodiment 27, wherein the dosing regimen comprises one to ten additional dosing cycles.

29. The method, mosunetuzumab for use, or use of embodiment 27 or 28, wherein the dosing regimen comprises ten additional dosing cycles.

30. The method, mosunetuzumab for use, or use of any one of embodiments 27-29, wherein the length of each of the one or more additional dosing cycles is 28 days.

31. The method, mosunetuzumab for use, or use of any one of embodiments 27-30, wherein each of the one or more additional dosing cycles comprises an additional dose of mosunetuzumab.

32. The method of embodiment 31, wherein each additional dose of mosunetuzumab is administered on Day 1 of each of the one or more additional dosing cycles.

33. The mosunetuzumab for use or use of embodiment 31, wherein each additional dose of mosunetuzumab is to be administered on Day 1 of each of the one or more additional dosing cycles.

34. The method of any one of embodiments 1-13, wherein administering the effective amount of mosunetuzumab comprises administering mosunetuzumab according to a dosing regimen comprising twelve dosing cycles, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second to twelfth dosing cycles each comprises a single dose (C2D1-C12D1) of mosunetuzumab, wherein each single dose C2D1-C12D1 are equivalent in amount, is equal to or greater than the C1D3, and is between 0.2 mg to 50 mg.

35. The mosunetuzumab for use or use of any one of embodiments 1-13, wherein the mosunetuzumab is to be administered to the subject according to a dosing regimen comprising twelve dosing cycles, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second to twelfth dosing cycles each comprises a single dose (C2D1-C12D1) of mosunetuzumab, wherein each single dose C2D1-C12D1 are equivalent in amount, is equal to or greater than the C1D3, and is between 0.2 mg to 50 mg.

36. The method, mosunetuzumab for use, or use of embodiment 34 or 35, wherein:
(a) the C1D1 is between 0.4 mg to 4.0 mg, the C1D2 is between 1.0 mg to 20.0 mg, and the C1D3 is between 3.0 mg to 50.0 mg; and
(b) each single dose C2D1-C12D1 is between 3.0 mg to 50.0 mg.

37. The method, mosunetuzumab for use, or use of any one of embodiments 34-36, wherein:
(a) the C1D1 is between 0.8 mg to 3.0 mg, the C1D2 is between 1.0 mg to 6.0 mg, and the C1D3 is between 3.0 mg to 45.0 mg; and
(b) each single dose C2D1-C12D1 is between 3.0 mg to 45.0 mg.

38. The method, mosunetuzumab for use, or use of any one of embodiments 34-37, wherein the C1D1 and C1D2 are each less than the C1D3.

39. The method, mosunetuzumab for use, or use of any one of embodiments 34-38, wherein the C1D2 is greater than the C1D1 by about 50% to about 250%.

40. The method, mosunetuzumab for use, or use of any one of embodiments 34-37, wherein:
(a) the C1D1 is 0.8 mg, the C1D2 is 2.0 mg, and the C1D3 is 4.2 mg, and each single dose C2D1-C12D1 is 4.2 mg;
(b) the C1D1 is 1.0 mg, the C1D2 is 1.0 mg, and the C1D3 is 3.0 mg, and each single dose C2D1-C12D1 is 30.0 mg; or
(c) the C1D1 is 1.0 mg, the C1D2 is 2.0 mg, and the C1D3 is 30.0 mg, and each single dose C2D1-C12D1 is 30.0 mg.

41. The method, mosunetuzumab for use, or use of any one of embodiments 34-40, wherein the length of the first dosing cycle is 21 days.

42. The method of embodiment 41, wherein the method comprises administering to the subject the C1D1, the C1D2, and the C1D3 on or about Days 1, 8, and 15, respectively, of the first dosing cycle.

43. The mosunetuzumab for use or use of embodiment 41, wherein the C1D1, the C1D2, and the C1D3 are to be administered to the subject on or about Days 1, 8, and 15, respectively, of the first dosing cycle.

44. The method, mosunetuzumab for use, or use of any one of embodiments 34-43, wherein the length of each of the second to twelfth dosing cycles is 28 days.

45. The method of embodiment 44, wherein the method comprises administering to the subject each of the C2D1-C12D1 on Day 1 of each respective dosing cycle.

46. The method of any one of embodiments 34-45, wherein the length of each of the second to twelfth dosing cycles is 28 days.

47. The method of any one of embodiments 34-43, wherein the length of each of the second to twelfth dosing cycles is 21 days.

48. The method of any of the preceding embodiments, wherein mosunetuzumab is administered intravenously.

49. The mosunetuzumab for use or use of any of the preceding embodiments, wherein mosunetuzumab is to be administered intravenously.

50. The method of any one of embodiments 14-49, wherein lenalidomide is administered during the second and subsequent cycles.

51. The mosunetuzumab for use or use of any one of embodiments 14-49, wherein lenalidomide is to be administered during the second and subsequent cycles.

52. The method of any one of embodiments 14-51, wherein lenalidomide is not administered during the first cycle.

53. The mosunetuzumab for use or use of any one of embodiments 14-51, wherein lenalidomide is not to be administered during the first cycle.

54. The method of any one of embodiments 14-53, wherein lenalidomide is administered daily.

55. The mosunetuzumab for use or use of any one of embodiments 14-53, wherein lenalidomide is to be administered daily.

56. The method of any one of embodiments 46 and 48-55, wherein lenalidomide is administered daily on the first 21 days of each dosing cycle comprising administration of lenalidomide.

57. The mosunetuzumab for use or use of any one of embodiments 46 and 48-55, wherein lenalidomide is to be administered daily on the first 21 days of each dosing cycle comprising administration of lenalidomide.

58. The method of any one of embodiments 47-55, wherein lenalidomide is administered daily on the first 14 days of each dosing cycle comprising administration of lenalidomide.

59. The mosunetuzumab for use or use of any one of embodiments 47-55, wherein lenalidomide is to be administered daily on the first 14 days of each dosing cycle comprising administration of lenalidomide.

60. The method of any one of embodiments 56-59, wherein lenalidomide is not administered on the last 7 days of each dosing cycle comprising administration of lenalidomide.

61. The mosunetuzumab for use or use of any one of embodiments 56-59, wherein lenalidomide is not to be administered on the last 7 days of each dosing cycle comprising administration of lenalidomide.

62. The method of any one of embodiments 14-61, wherein lenalidomide is administered at a dose of 20 mg.

63. The mosunetuzumab for use or use of any one of embodiments 14-61, wherein lenalidomide is to be administered at a dose of 20 mg.

64. The method of any of the preceding embodiments, wherein lenalidomide is administered orally.

65. The mosunetuzumab for use or use of any of the preceding embodiments, wherein lenalidomide is to be administered orally. 66. The method, mosunetuzumab for use, or use of any of the preceding embodiments, wherein the subject was previously treated with at least one anti-CD20 monoclonal antibody. 67. The method, mosunetuzumab for use, or use of embodiment 66, wherein the subject is relapsed or refractory to the treatment comprising the anti-CD20 monoclonal antibody. 68. The method, mosunetuzumab for use, or use of embodiment 66 or 67, wherein the anti-CD20 monoclonal antibody is obinutuzumab or rituximab. 69. A method of treating a subject, comprising administering to the subject an effective amount of mosunetuzumab and an effective amount of lenalidomide,

wherein the subject:
(i) has relapsed or refractory follicular lymphoma (R/R FL) that expresses CD20, and
(ii) has been previously treated with at least one chemo-immunotherapy regimen; and

wherein administering the effective amount of mosunetuzumab and lenalidomide comprises administering mosunetuzumab and lenalidomide according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and
(c) the second dosing cycle further comprises orally administering 20 mg lenalidomide daily on Days 1-21 of the second dosing cycle.

70. Mosunetuzumab for use in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

71. Use of mosunetuzumab in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

72. Use of mosunetuzumab in the manufacture of a medicament for use in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

73. Use of lenalidomide in the manufacture of a medicament for use in combination with mosunetuzumab for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

74. Use of mosunetuzumab and lenalidomide in the manufacture of a medicament for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

75. A method of treating a subject, comprising administering to the subject an effective amount of mosunetuzumab and an effective amount of lenalidomide,

wherein the subject:
(i) has relapsed or refractory follicular lymphoma (R/R FL) that expresses CD20, and
(ii) has been previously treated with at least one chemo-immunotherapy regimen; and

wherein administering the effective amount of mosunetuzumab and lenalidomide comprises administering mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-21 of each dosing cycle.

76. Mosunetuzumab for use in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

77. Use of mosunetuzumab in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

78. Use of mosunetuzumab in the manufacture of a medicament for use in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

79. Use of lenalidomide in the manufacture of a medicament for use in combination with mosunetuzumab for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

80. Use of mosunetuzumab and lenalidomide in the manufacture of a medicament for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

81. A method of treating a subject, comprising administering to the subject an effective amount of mosunetuzumab and an effective amount of lenalidomide,

wherein the subject:
(i) has relapsed or refractory follicular lymphoma (R/R FL) that expresses CD20, and
(ii) has been previously treated with at least one chemo-immunotherapy regimen; and

wherein administering the effective amount of mosunetuzumab and lenalidomide comprises administering mosunetuzumab and lenalidomide according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-14 of the second dosing cycle.

82. Mosunetuzumab for use in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

83. Use of mosunetuzumab in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

84. Use of mosunetuzumab in the manufacture of a medicament for use in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

85. Use of lenalidomide in the manufacture of a medicament for use in combination with mosunetuzumab for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

86. Use of mosunetuzumab and lenalidomide in the manufacture of a medicament for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

87. A method of treating a subject, comprising administering to the subject an effective amount of mosunetuzumab and an effective amount of lenalidomide,

wherein the subject:
(i) has relapsed or refractory follicular lymphoma (R/R FL) that expresses CD20, and
(ii) has been previously treated with at least one chemo-immunotherapy regimen that included obinutuzumab; and

wherein administering the effective amount of mosunetuzumab and lenalidomide comprises administering mosunetuzumab and lenalidomide according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-14 of each dosing cycle.

88. Mosunetuzumab for use in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

89. Use of mosunetuzumab in combination with lenalidomide in treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

90. Use of mosunetuzumab in the manufacture of a medicament for use in combination with lenalidomide for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

91. Use of lenalidomide in the manufacture of a medicament for use in combination with mosunetuzumab for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

92. Use of mosunetuzumab and lenalidomide in the manufacture of a medicament for treating a subject, wherein the subject:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the subject according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

93, The method, mosunetuzumab for use, or use of any one of embodiments 69-92, wherein at least one chemo-immunotherapy regimen comprises an anti-CD20 monoclonal antibody.

94. The method, mosunetuzumab for use, or use of embodiment 93, wherein the anti-CD20 monoclonal antibody is rituximab or obinutuzumab.

95. The method, mosunetuzumab for use, or use of any one of embodiments 69-94, wherein the subject has received only one prior line of systemic therapy and either:
(a) has a Follicular Lymphoma International Prognostic Index (FLIPI; Solal-Céligny et al. Blood. 2004; 104 (5): 1258-1265.) score of 2-5,
(b) has been refractory to prior obinutuzumab treatment,
(c) has been refractory to prior rituximab treatment, or
(d) has disease progression within 24 months after initiation of prior therapy.

96. The method of any one of embodiments 93-95, wherein the subject has not been treated with obinutuzumab or rituximab for at least 4 weeks prior to being administered the effective amount of mosunetuzumab and lenalidomide.

97. The mosunetuzumab for use or use of any one of embodiments 93-95, wherein mosunetuzumab and lenalidomide are not to be administered to the subject for at least 4 weeks after the subject has received treatment with an anti-CD20 monoclonal antibody.

98. The method, mosunetuzumab for use, or use of any one of embodiments 69-92, wherein mosunetuzumab and lenalidomide have a synergistic effect on the R/R FL.

99. The method, mosunetuzumab for use, or use of embodiment 98, wherein the synergistic effect is a partial response, as defined by Lugano 2014 criteria (Cheson B D, et al. J Clin Oncol 2014; 32:1-9).

100. The method, mosunetuzumab for use, or use of embodiment 98, wherein the synergistic effect is a complete response, as defined by Lugano 2014 criteria (Cheson B D, et al. J Clin Oncol 2014; 32:1-9).

101. The method, mosunetuzumab for use, or use of any of the preceding embodiments, wherein the FL is histologically documented as Grade 1, 2, or 3a, but not 3b according to the World Health Organization classification of lymphoid neoplasms (as referenced in Swerdlow S H, et al. Blood 2016; 127:2375-90).

102. A method of treating a population of subjects, comprising administering to each subject in the population an effective amount of mosunetuzumab and an effective amount of lenalidomide,

wherein each subject:
(a) has relapsed or refractory follicular lymphoma (R/R FL) that expresses CD20, and
(b) has been previously treated with at least one chemo-immunotherapy regimen; and

wherein administering the effective amount of mosunetuzumab and lenalidomide comprises administering mosunetuzumab and lenalidomide according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and
(c) the second dosing cycle further comprises orally administering 20 mg lenalidomide daily on Days 1-21 of the second dosing cycle.

103. Mosunetuzumab for use in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

104. Use of mosunetuzumab in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

105. Use of mosunetuzumab in the manufacture of a medicament for use in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

106. Use of lenalidomide in the manufacture of a medicament for use in combination with mosunetuzumab for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

107. Use of mosunetuzumab and lenalidomide in the manufacture of a medicament for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-21 of the second dosing cycle.

108. A method of treating a population of subjects, comprising administering to each subject in the population an effective amount of mosunetuzumab and an effective amount of lenalidomide,

wherein each subject:
(a) has relapsed or refractory follicular lymphoma (R/R FL) that expresses CD20, and
(b) has been previously treated with at least one chemo-immunotherapy regimen that included an anti-CD20 monoclonal antibody; and

wherein administering the effective amount of mosunetuzumab and lenalidomide comprises administering mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprises a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-21 of each dosing cycle.

109. Mosunetuzumab for use in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

110. Use of mosunetuzumab in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

111. Use of mosunetuzumab in the manufacture of a medicament for use in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

112. Use of lenalidomide in the manufacture of a medicament for use in combination with mosunetuzumab for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

113. Use of mosunetuzumab and lenalidomide in the manufacture of a medicament for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered orally on Days 1-21 of each dosing cycle.

114. A method of treating a population of subjects, comprising administering to each subject in the population an effective amount of mosunetuzumab and an effective amount of lenalidomide,

wherein each subject:
(a) has relapsed or refractory follicular lymphoma (R/R FL) that expresses CD20, and
(b) has been previously treated with at least one chemo-immunotherapy regimen that included an anti-CD20 monoclonal antibody; and

wherein administering the effective amount of mosunetuzumab and lenalidomide comprises administering mosunetuzumab and lenalidomide according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and (c) the second dosing cycle further comprises orally administering 20 mg lenalidomide daily on Days 1-14 of the second dosing cycle.

115. Mosunetuzumab for use in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

116. Use of mosunetuzumab in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

117. Use of mosunetuzumab in the manufacture of a medicament for use in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

118. Use of lenalidomide in the manufacture of a medicament for use in combination with mosunetuzumab for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

119. Use of mosunetuzumab and lenalidomide in the manufacture of a medicament for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising at least a first 21-day dosing cycle and a second 21-day dosing cycle, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab to be administered intravenously on Day 1 of the second dosing cycle, wherein the C2D1 is 30 mg, and
(c) the second dosing cycle further comprises daily doses of 20 mg lenalidomide to be administered orally on Days 1-14 of the second dosing cycle.

120. A method of treating a population of subjects, comprising administering to each subject in the population an effective amount of mosunetuzumab and an effective amount of lenalidomide,

wherein each subject:
(a) has relapsed or refractory follicular lymphoma (R/R FL) that expresses CD20, and
(b) has been previously treated with at least one chemo-immunotherapy regimen that included an anti-CD20 monoclonal antibody; and

wherein administering the effective amount of mosunetuzumab and lenalidomide comprises administering mosunetuzumab and lenalidomide according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprises a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-14 of each dosing cycle.

121. Mosunetuzumab for use in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

122. Use of mosunetuzumab in combination with lenalidomide in treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

123. Use of mosunetuzumab in the manufacture of a medicament for use in combination with lenalidomide for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

124. Use of lenalidomide in the manufacture of a medicament for use in combination with mosunetuzumab for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

125. Use of mosunetuzumab and lenalidomide in the manufacture of a medicament for treating a population of subjects, wherein each subject in the population:
(i) has relapsed or refractory follicular lymphoma (R/R FL) that expresses CD20, and
(ii) has been previously treated with at least one chemo-immunotherapy regimen; and

wherein mosunetuzumab and lenalidomide are to be administered to the population of subjects according to a dosing regimen comprising a first 21-day dosing cycle and eleven subsequent 21-day dosing cycles, wherein:
(a) the first dosing cycle comprises a first dose (C1D1), a second dose (C1D2), and a third dose (C1D3) of mosunetuzumab to be administered intravenously on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab to be administered intravenously on Day 1 of each dosing cycle, wherein each single dose C2D1-C12D1 is 30 mg, and
(c) the second to twelfth dosing cycles each further comprises daily doses of 20 mg lenalidomide to be administered on Days 1-14 of each dosing cycle.

126. The method, mosunetuzumab for use, or use of any one of embodiments 102-125, wherein at least one chemo-immunotherapy regimen comprises an anti-CD20 monoclonal antibody.

127. The method, mosunetuzumab for use, or use of embodiment 126, wherein the anti-CD20 monoclonal antibody is rituximab or obinutuzumab.

128. The method, mosunetuzumab for use, or use of any one of embodiments 102-127, wherein each subject has received only one prior line of systemic therapy and either:
(a) has a Follicular Lymphoma International Prognostic Index (FLIPI) score of 2-5,
(b) has been refractory to prior anti-CD20 monoclonal antibody treatment, or
(c) has disease progression within 24 months after initiation of prior therapy.

129. The method, mosunetuzumab for use, or use of any one of embodiments 102-128, wherein the FL of each subject is histologically documented as Grade 1, 2, or 3a, but not 3b according to the World Health Organization classification of lymphoid neoplasms (as referenced in Swerdlow S H, et al. Blood 2016; 127:2375-90).

130. The method of any one of embodiments 102-129, wherein each subject has not been treated with the anti-CD20 monoclonal antibody for at least 4 weeks prior to being administered the effective amount of mosunetuzumab and lenalidomide.

131. The mosunetuzumab for use or use of any one of embodiments 102-129, wherein mosunetuzumab and lenalidomide are not to be administered to each subject for at least 4 weeks after said subject has received treatment with an anti-CD20 monoclonal antibody.

132. The method of any one of embodiments 102-125, wherein mosunetuzumab and lenalidomide have a synergistic effect on the R/R FL.

133. The method of embodiment 132, wherein the synergistic effect is a partial response, as defined by Lugano 2014 criteria (Cheson B D, et al. J Clin Oncol 2014; 32:1-9).

134. The method of embodiment 132, wherein the synergistic effect is a complete response, as defined by Lugano 2014 criteria (Cheson B D, et al. J Clin Oncol 2014; 32:1-9).

135. The method, mosunetuzumab for use, or use of any one of embodiments 102-134, wherein the incidence of an adverse event is not significantly higher than if mosunetuzumab was administered alone to the population of subjects.

136. The method, mosunetuzumab for use, or use of any one of embodiments 102-134, wherein the incidence of an adverse event is not significantly higher than if lenalidomide is not administered to the population of subjects.

137. The method, mosunetuzumab for use, or use of any one of embodiments 102-136, wherein the incidence rate of cytokine release syndrome as defined by the American Society of Transplantation and Cellular Therapy (ASTCT) Consensus Grading for Cytokine-Release Syndrome (“ASTCT CRS grading;” Lee et al., Biol Blood Marrow Transplant 2019) is less than 45%.

138. The method, mosunetuzumab for use, or use of embodiment 137, wherein the incidence rate of cytokine release syndrome as defined by the American Society of Transplantation and Cellular Therapy (ASTCT) Consensus Grading for Cytokine-Release Syndrome (“ASTCT CRS grading”) is less than 35%.

139. The method, mosunetuzumab for use, or use of embodiment 138, wherein the incidence rate of cytokine release syndrome as defined by the American Society of Transplantation and Cellular Therapy (ASTCT) Consensus Grading for Cytokine-Release Syndrome (“ASTCT CRS grading”) is less than 25%.

140. The method, mosunetuzumab for use, or use of any one of embodiments 102-139, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 10%.

141. The method, mosunetuzumab for use, or use of embodiment 140, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 5%.

142. The method, mosunetuzumab for use, or use of embodiment 141, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 3%.

143. The method, mosunetuzumab for use, or use of embodiment 142, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 1%.

144. The method, mosunetuzumab for use, or use of any one of embodiments 102-143, wherein the incidence rate of neutropenia is less than 40%.

145. The method, mosunetuzumab for use, or use of embodiment 144, wherein the incidence rate of neutropenia is less than 30%.

146. The method, mosunetuzumab for use, or use of embodiment 145, wherein the incidence rate of neutropenia is less than 20%.

147. The method, mosunetuzumab for use, or use of any one of embodiments 102-146, wherein the overall response rate is at least 80%.

148. The method, mosunetuzumab for use, or use of embodiment 147, wherein the overall response rate is at least 90%.

149. The method, mosunetuzumab for use, or use of embodiment 148, wherein the overall response rate is at least 95%.

150. The method, mosunetuzumab for use, or use of embodiment 149, wherein the overall response rate is at least 99%.

151. The method, mosunetuzumab for use, or use of any one of embodiments 102-146, wherein the complete response rate is at least 65%.

152. The method, mosunetuzumab for use, or use of embodiment 151, wherein the complete response rate is at least 75%.

153. The method, mosunetuzumab for use, or use of embodiment 152, wherein the complete response rate is at least 85%.

154. The method, mosunetuzumab for use, or use of any one of embodiments 102-153, wherein the anti-CD20 monoclonal antibody is obinutuzumab or rituximab.

155. The method, mosunetuzumab for use, or use of any one of embodiments 1-101, wherein the subject is human.

156. The method, mosunetuzumab for use, or use of any one of embodiments 102-154, wherein each subject in the population is human.

157. The method of any one of embodiments 1-101, wherein the subject exhibits a reduction in tumor burden after being administered an effective amount of mosunetuzumab and an effective amount of lenalidomide.

158. The mosunetuzumab for use or use of any one of embodiments 1-101, wherein the subject exhibits a reduction in tumor burden after being treated with mosunetuzumab and lenalidomide.

159. The method, mosunetuzumab for use, or use of embodiment 157 or 158, wherein the reduction in tumor burden is determined by computed tomography (CT).

160. The method, mosunetuzumab for use, or use of any one of embodiments 157-159, wherein the reduction in tumor burden is a decrease in the sum of the product of the diameters (SPD) of target lesions.

161. The method, mosunetuzumab for use, or use of embodiment 160, wherein the decrease in SPD is at least 40%.

162. The method, mosunetuzumab for use, or use of embodiment 161, wherein the decrease in SPD is at least 60%.

163. The method, mosunetuzumab for use, or use of embodiment 162, wherein the decrease in SPD is at least 80%.

164. The method, mosunetuzumab for use, or use of any one of embodiments 102-154, wherein at least 45% of subjects in the population exhibit a reduction in tumor burden after being administered mosunetuzumab and lenalidomide.

165. The method, mosunetuzumab for use, or use of embodiment 164, wherein at least 60% of subjects in the population exhibit a reduction in tumor burden after being administered mosunetuzumab and lenalidomide.

166. The method, mosunetuzumab for use, or use of embodiment 165, wherein at least 75% of subjects in the population exhibit a reduction in tumor burden after being administered an effective amount of mosunetuzumab and an effective amount of lenalidomide.

167. The method, mosunetuzumab for use, or use of any one of embodiments 164-166, wherein the reduction in tumor burden is determined by computed tomography (CT).

168. The method, mosunetuzumab for use, or use of any one of embodiments 164-167, wherein the reduction in tumor burden is a decrease in the sum of the product of the diameters (SPD) of target lesions.

169. The method, mosunetuzumab for use, or use of embodiment 168, wherein the decrease in SPD is at least 40%.

170. The method, mosunetuzumab for use, or use of embodiment 169, wherein the decrease in SPD is at least 60%.

171. The method, mosunetuzumab for use, or use of embodiment 170, wherein the decrease in SPD is at least 80%.

172. The method, mosunetuzumab for use, or use of any one of embodiments 8-54, wherein the dosing regimen further comprises administration of a corticosteroid.

173. The method, mosunetuzumab for use, or use of embodiment 172, wherein the corticosteroid is administered or is to be administered to the subject during the first dosing cycle.

174. The method, mosunetuzumab for use, or use of embodiment 173, wherein the first dosing cycle comprises a first dose (C1D1), a second dose (C1D2), and a third dose (C1D3) of the corticosteroid.

175. The method, mosunetuzumab for use, or use of embodiment 174, wherein the C1D1, the C1D2, and the C1D3 of the corticosteroid are administered or are to be administered to the subject on Days 1, 8, and 15, respectively, of the first dosing cycle.

176. The method, mosunetuzumab for use, or use of embodiment 175, wherein each single dose C1D1-C1D3 of the corticosteroid is administered or is to be administered to the subject before administration of the C1D1-C1D3, respectively, of mosunetuzumab.

177. The method, mosunetuzumab for use, or use of any one of embodiments 172-176, wherein the corticosteroid is administered or is to be administered to the subject on the first dosing cycle and not on the second dosing cycle.

178. The method, mosunetuzumab for use, or use of any one of embodiments 172-176, wherein the corticosteroid is administered or is to be administered to the subject on the second dosing cycle.

179. The method, mosunetuzumab for use, or use of embodiment 178, wherein the second dosing cycle comprises a single dose (C2D1) of the corticosteroid.

180. The method, mosunetuzumab for use, or use of embodiment 179, wherein the C2D1 of the corticosteroid is administered or is to be administered to the subject on Day 1 of the second dosing cycle.

181. The method, mosunetuzumab for use, or use of embodiment 180, wherein the C2D1 of the corticosteroid is administered or is to be administered to the subject before administration of the C2D1 of mosunetuzumab.

182. The method, mosunetuzumab for use, or use of any one of embodiments 102-154, wherein the dosing regimen further comprises administration of a corticosteroid.

183. The method, mosunetuzumab for use, or use of embodiment 182, wherein the corticosteroid is administered or is to be administered to the subjects during the first dosing cycle.

184. The method, mosunetuzumab for use, or use of embodiment 183, wherein the first dosing cycle comprises a first dose (C1D1), a second dose (C1D2), and a third dose (C1D3) of the corticosteroid.

185. The method, mosunetuzumab for use, or use of embodiment 184, wherein the C1D1, the C1D2, and the C1D3 of the corticosteroid are administered or are to be administered to the subjects on Days 1, 8, and 15, respectively, of the first dosing cycle.

186. The method, mosunetuzumab for use, or use of embodiment 185, wherein each single dose C1D1-C1D3 of the corticosteroid is administered or is to be administered to the subjects before administration of the C1D1-C1D3, respectively, of mosunetuzumab.

187. The method, mosunetuzumab for use, or use of any one of embodiments 182-186, wherein the corticosteroid is administered or is to be administered to the subjects on the first dosing cycle and not on the second dosing cycle.

188. The method, mosunetuzumab for use, or use of any one of embodiments 182-186, wherein the corticosteroid is administered or is to be administered to the subjects on the second dosing cycle.

189. The method, mosunetuzumab for use, or use of embodiment 188, wherein the second dosing cycle comprises a single dose (C2D1) of the corticosteroid.

190. The method, mosunetuzumab for use, or use of embodiment 189, wherein the C2D1 of the corticosteroid is administered or is to be administered to the subjects on Day 1 of the second dosing cycle.

191. The method, mosunetuzumab for use, or use of embodiment 190, wherein the C2D1 of the corticosteroid is administered or is to be administered to the subjects before administration of the C2D1 of mosunetuzumab.

192. The method, mosunetuzumab for use, or use of any one of embodiments 31-33, wherein each additional dosing cycle comprises administration of an additional dose of the corticosteroid.

193. The method, mosunetuzumab for use, or use of embodiment 192, wherein each additional dose of the corticosteroid is administered or is to be administered on Day 1 of each additional dosing cycle.

194. The method, mosunetuzumab for use, or use of embodiment 193, wherein each additional dose of the corticosteroid is administered or is to be administered to the subject before administration of each additional dose of mosunetuzumab.

195. The method, mosunetuzumab for use, or use of any one of embodiments 172-194, wherein the corticosteroid is administered or is to be administered intravenously.

196. The method, mosunetuzumab for use, or use of any one of embodiments 172-195, wherein the corticosteroid is dexamethasone.

197. The method, mosunetuzumab for use, or use of embodiment 196, wherein each dose of dexamethasone is 10 mg.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating a subject, comprising administering to the subject an effective amount of mosunetuzumab and an effective amount of lenalidomide, wherein the subject:
(a) has relapsed or refractory follicular lymphoma (R/R FL) that expresses CD20, and
(b) has been previously treated with at least one chemo-immunotherapy regimen.

2. The method of claim 1, wherein at least one chemo-immunotherapy regimen comprises an anti-CD20 monoclonal antibody.

3. The method of claim 1 or 2, wherein the subject has received only one prior line of systemic therapy and either:
(a) has a Follicular Lymphoma International Prognostic Index (FLIPI; Solal-Céligny et al. Blood. 2004; 104 (5): 1258-1265.) score of 2-5,
(b) has been refractory to prior anti-CD20 monoclonal antibody treatment, or
(c) has disease progression within 24 months after initiation of prior therapy.

4. The method of claim 2 or 3, wherein the subject has not been treated with the anti-CD20 monoclonal antibody for at least 4 weeks prior to being administered the effective amount of mosunetuzumab and lenalidomide.

5. The method of claim 1, wherein mosunetuzumab and lenalidomide have a synergistic effect on the R/R FL.

6. The method of claim 5, wherein the synergistic effect is a partial response, as defined by Lugano 2014 criteria (Cheson B D, et al. J Clin Oncol 2014; 32:1-9).

7. The method of claim 5, wherein the synergistic effect is a complete response, as defined by Lugano 2014 criteria (Cheson B D, et al. J Clin Oncol 2014; 32:1-9).

8. The method of any of the preceding claims, wherein administering the effective amount of mosunetuzumab comprises administering mosunetuzumab according to a dosing regimen comprising at least a first dosing cycle and a second dosing cycle, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second dosing cycle comprises a single dose (C2D1) of mosunetuzumab, wherein the C2D1 is equal to or greater than the C1D3 and is between 0.2 mg to 50 mg.

9. The method of claim 8, wherein:
(a) the C1D1 is between 0.4 mg to 4.0 mg, the C1D2 is between 1.0 mg to 20.0 mg, and the C1D3 is between 3.0 mg to 50.0 mg; and
(b) the C2D1 is between 3.0 mg to 50.0 mg.

10. The method of claim 8 or 9, wherein:
(a) the C1D1 is between 0.8 mg to 3.0 mg, the C1D2 is between 1.0 mg to 6.0 mg, and the C1D3 is between 3.0 mg to 45.0 mg; and
(b) the C2D1 is between 3.0 mg to 45.0 mg.

11. The method of any one of claims 8-10, wherein the C1D1 and C1D2 are each less than the C1D3.

12. The method of any one of claims 8-10, wherein the C1D2 is greater than the C1D1 by about 50% to about 250%.

13. The method of any one of claims 8-10, wherein:
(a) the C1D1 is 0.8 mg, the C1D2 is 2.0 mg, and the C1D3 is 4.2 mg, and the C2D1 is 4.2 mg;
(b) the C1D1 is 1.0 mg, the C1D2 is 1.0 mg, and the C1D3 is 3.0 mg, and the C2D1 is 30.0 mg; or
(c) the C1D1 is 1.0 mg, the C1D2 is 2.0 mg, and the C1D3 is 30.0 mg, and the C2D1 is 30.0 mg.

14. The method of any one of claims 8-13, wherein the length of the first dosing cycle is 21 days.

15. The method of claim 14, wherein the method comprises administering to the subject the C1D1, the C1D2, and the C1D3 on or about Days 1, 8, and 15, respectively, of the first dosing cycle.

16. The method of any one of claims 8-15, wherein the length of the second dosing cycle is 28 days.

17. The method of claim 16, wherein the method comprises administering to the subject the C2D1 on Day 1 of the second dosing cycle.

18. The method of any one of claims 8-17, wherein the dosing regimen comprises one or more additional dosing cycles.

19. The method of claim 18, wherein the dosing regimen comprises one to ten additional dosing cycles.

20. The method of claim 18 or 19, wherein the dosing regimen comprises ten additional dosing cycles.

21. The method of any one of claims 18-20, wherein the length of each of the one or more additional dosing cycles is 28 days.

22. The method of any one of claims 18-21, wherein each of the one or more additional dosing cycles comprises an additional dose of mosunetuzumab.

23. The method of claim 22, wherein the method comprises administering to the subject each additional dose of mosunetuzumab on Day 1 of each of the one or more additional dosing cycles.

24. The method of any one of claims 1-7, wherein administering the effective amount of mosunetuzumab comprises administering mosunetuzumab according to a dosing regimen comprising twelve dosing cycles, wherein:
(a) the first dosing cycle comprises a first dose (C1D1), a second dose (C1D2), and a third dose (C1D3) of mosunetuzumab, wherein the C1D1 and the C1D2 are each no greater than the C1D3, and wherein the C1D1 is between 0.02 mg to 4.0 mg, the C1D2 is between 0.05 mg to 20.0 mg, and the C1D3 is between 0.2 mg to 50.0 mg; and
(b) the second to twelfth dosing cycles each comprises a single dose (C2D1-C12D1) of mosunetuzumab, wherein each single dose C2D1-C12D1 are equivalent in amount, is equal to or greater than the C1D3, and is between 0.2 mg to 50 mg.

25. The method of claim 24, wherein:
(a) the C1D1 is between 0.4 mg to 4.0 mg, the C1D2 is between 1.0 mg to 20.0 mg, and the C1D3 is between 3.0 mg to 50.0 mg; and
(b) each single dose C2D1-C12D1 is between 3.0 mg to 50.0 mg.

26. The method of claim 24 or 25, wherein:
(a) the C1D1 is between 0.8 mg to 3.0 mg, the C1D2 is between 1.0 mg to 6.0 mg, and the C1D3 is between 3.0 mg to 45.0 mg; and
(b) each single dose C2D1-C12D1 is between 3.0 mg to 45.0 mg.

27. The method of any one of claims 24-26, wherein the C1D1 and C1D2 are each less than the C1D3.

28. The method of any one of claims 24-26, wherein the C1D2 is greater than the C1D1 by about 50% to about 250%.

29. The method of any one of claims 24-26, wherein:
(a) the C1D1 is 0.8 mg, the C1D2 is 2.0 mg, and the C1D3 is 4.2 mg, and each single dose C2D1-C12D1 is 4.2 mg;
(b) the C1D1 is 1.0 mg, the C1D2 is 1.0 mg, and the C1D3 is 3.0 mg, and each single dose C2D1-C12D1 is 30.0 mg; or
(c) the C1D1 is 1.0 mg, the C1D2 is 2.0 mg, and the C1D3 is 30.0 mg, and each single dose C2D1-C12D1 is 30.0 mg.

30. The method of any one of claims 24-29, wherein the length of the first dosing cycle is 21 days.

31. The method of claim 30, wherein the method comprises administering to the subject the C1D1, the C1D2, and the C1D3 on or about Days 1, 8, and 15, respectively, of the first dosing cycle.

32. The method of any one of claims 24-31, wherein the length of each of the second to twelfth dosing cycles is 28 days.

33. The method of claim 32, wherein the method comprises administering to the subject each of the C2D1-C12D1 on Day 1 of each respective dosing cycle.

34. The method of any one of claims 24-33, wherein the length of each of the second to twelfth dosing cycles is 28 days.

35. The method of any of the preceding claims, wherein mosunetuzumab is administered intravenously.

36. The method of any one of claims 8-35, wherein lenalidomide is administered during the second and subsequent cycles.

37. The method of any one of claims 8-36, wherein lenalidomide is not administered during the first cycle.

38. The method of any one of claims 8-37, wherein lenalidomide is administered daily.

39. The method of any one of claims 36-38, wherein lenalidomide is administered daily on the first 21 days of each dosing cycle comprising administration of lenalidomide.

40. The method of claim 39, wherein lenalidomide is not administered on the last 7 days of each dosing cycle comprising administration of lenalidomide.

41. The method of any one of claims 8-40, wherein lenalidomide is administered at a dose of 20 mg.

42. The method of any of the preceding claims, wherein lenalidomide is administered orally.

43. The method of any of the preceding claims, wherein the subject was previously treated with at least one anti-CD20 monoclonal antibody.

44. The method of claim 43, wherein the subject is relapsed or refractory to the treatment comprising the anti-CD20 monoclonal antibody.

45. The method of claim 43 or 44, wherein the anti-CD20 monoclonal antibody is obinutuzumab or rituximab.

46. A method of treating a subject, comprising administering to the subject an effective amount of mosunetuzumab and an effective amount of lenalidomide,
wherein the subject:
(i) has relapsed or refractory follicular lymphoma (R/R FL) that expresses CD20, and
(ii) has been previously treated with at least one chemo-immunotherapy regimen; and
wherein administering the effective amount of mosunetuzumab and lenalidomide comprises administering mosunetuzumab and lenalidomide according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and
(c) the second dosing cycle further comprises orally administering 20 mg lenalidomide daily on Days 1-21 of the second dosing cycle.

47. A method of treating a subject, comprising administering to the subject an effective amount of mosunetuzumab and an effective amount of lenalidomide,
wherein the subject:
(i) has relapsed or refractory follicular lymphoma (R/R FL) that expresses CD20, and
(ii) has been previously treated with at least one chemo-immunotherapy regimen; and
wherein administering the effective amount of mosunetuzumab and lenalidomide comprises administering mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprise a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-21 of each dosing cycle.

48. The method of any one of claims 45-47, wherein at least one chemo-immunotherapy regimen comprises an anti-CD20 monoclonal antibody.

49. The method of any one of claims 45-48, wherein the subject has received only one prior line of systemic therapy and either:
(a) has a Follicular Lymphoma International Prognostic Index (FLIPI) score of 2-5,
(b) has been refractory to prior obinutuzumab treatment,
(c) has been refractory to prior rituximab treatment, or
(d) has disease progression within 24 months after initiation of prior therapy.

50. The method of any one of claims 45-49, wherein the subject has not been treated with obinutuzumab or rituximab for at least 4 weeks prior to being administered the effective amount of mosunetuzumab and lenalidomide.

51. The method of any one of claims 45-49, wherein mosunetuzumab and lenalidomide have a synergistic effect on the R/R FL.

52. The method of claim 51, wherein the synergistic effect is a partial response, as defined by Lugano 2014 criteria (Cheson B D, et al. J Clin Oncol 2014; 32:1-9).

53. The method of claim 51, wherein the synergistic effect is a complete response, as defined by Lugano 2014 criteria (Cheson B D, et al. J Clin Oncol 2014; 32:1-9).

54. The method of any of the preceding claims, wherein the FL is histologically documented as Grade 1, 2, or 3a, but not 3b according to the World Health Organization classification of lymphoid neoplasms (as referenced in Swerdlow S H, et al. Blood 2016; 127:2375-90).

55. A method of treating a population of subjects, comprising administering to each subject in the population an effective amount of mosunetuzumab and an effective amount of lenalidomide,
wherein each subject:
(a) has relapsed or refractory follicular lymphoma (R/R FL) that expresses CD20, and
(b) has been previously treated with at least one chemo-immunotherapy regimen; and
wherein administering the effective amount of mosunetuzumab and lenalidomide comprises administering mosunetuzumab and lenalidomide according to a dosing regimen comprising at least a first 21-day dosing cycle and a second 28-day dosing cycle, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second dosing cycle comprises a single dose (C2D1) of mosunetuzumab intravenously administered on Day 1 of the second dosing cycle, wherein the C2D1 is 30 mg, and
(c) the second dosing cycle further comprises orally administering 20 mg lenalidomide daily on Days 1-21 of the second dosing cycle.

56. A method of treating a population of subjects, comprising administering to each subject in the population an effective amount of mosunetuzumab and an effective amount of lenalidomide,
wherein each subject:
(a) has relapsed or refractory follicular lymphoma (R/R FL) that expresses CD20, and
(b) has been previously treated with at least one chemo-immunotherapy regimen; and
wherein administering the effective amount of mosunetuzumab and lenalidomide comprises administering mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab intravenously administered on Days 1, 8, and 15, respectively, of the first dosing cycle, wherein the C1D1 is 1 mg, the C1D2 is 2 mg, and the C1D3 is 30 mg,
(b) the second to twelfth dosing cycles each comprises a single dose (C2D1-C12D1) of mosunetuzumab intravenously administered on Day 1 of each dosing cycle, wherein each single dose C2D1-C12D1 is 30 mg, and
(c) the second to twelfth dosing cycles each further comprises orally administering 20 mg lenalidomide daily on Days 1-21 of each dosing cycle.

57. The method of claim 55 or 56, wherein at least one chemo-immunotherapy regimen comprises an anti-CD20 monoclonal antibody.

58. The method of any one of claims 55-57, wherein each subject has received only one prior line of systemic therapy and either:
(a) has a Follicular Lymphoma International Prognostic Index (FLIPI) score of 2-5,
(b) has been refractory to prior anti-CD20 monoclonal antibody treatment, or
(c) has disease progression within 24 months after initiation of prior therapy.

59. The method of any one of claims 55-58, wherein the FL of each subject is histologically documented as Grade 1, 2, or 3a, but not 3b according to the World Health Organization classification of lymphoid neoplasms (as referenced in Swerdlow S H, et al. Blood 2016; 127:2375-90).

60. The method of any one of claims 57-59, wherein each subject has not been treated with the anti-CD20 monoclonal antibody for at least 4 weeks prior to being administered the effective amount of mosunetuzumab and lenalidomide.

61. The method of claim 60, wherein mosunetuzumab and lenalidomide have a synergistic effect on the R/R FL.

62. The method of claim 61, wherein the synergistic effect is a partial response, as defined by Lugano 2014 criteria (Cheson B D, et al. J Clin Oncol 2014; 32:1-9).

63. The method of claim 61, wherein the synergistic effect is a complete response, as defined by Lugano 2014 criteria (Cheson B D, et al. J Clin Oncol 2014; 32:1-9).

64. The method of any one of claims 55-63, wherein the incidence of an adverse event is not significantly higher than if mosunetuzumab was administered alone to the population of subjects.

65. The method of any one of claims 55-63, wherein the incidence of an adverse event is not significantly higher than if lenalidomide is not administered to the population of subjects.

66. The method of any one of claims 55-65, wherein the incidence rate of cytokine release syndrome as defined by the American Society of Transplantation and Cellular Therapy (ASTCT) Consensus Grading for Cytokine-Release Syndrome (“ASTCT CRS grading;” Lee et al., Biol Blood Marrow Transplant 2019) is less than 45%.

67. The method of claim 66, wherein the incidence rate of cytokine release syndrome as defined by the American Society of Transplantation and Cellular Therapy (ASTCT) Consensus Grading for Cytokine-Release Syndrome (“ASTCT CRS grading”) is less than 35%.

68. The method of claim 67, wherein the incidence rate of cytokine release syndrome as defined by the American Society of Transplantation and Cellular Therapy (ASTCT) Consensus Grading for Cytokine-Release Syndrome (“ASTCT CRS grading”) is less than 25%.

69. The method of any one of claims 55-68, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 10%.

70. The method of claim 69, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 5%.

71. The method of claim 70, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 3%.

72. The method of claim 71, wherein the incidence rate of cytokine release syndrome having a grade of 3 or higher as defined by the American Society of Transplantation and Cellular Therapy (ASTCT) Consensus Grading for Cytokine-Release Syndrome (“ASTCT CRS grading”) is less than 1%.

73. The method of any one of claims 55-72, wherein the incidence rate of neutropenia is less than 40%.

74. The method of claim 73, wherein the incidence rate of neutropenia is less than 30%.

75. The method of claim 74, wherein the incidence rate of neutropenia is less than 20%.

76. The method of any one of claims 55-75, wherein the overall response rate is at least 80%.

77. The method of claim 76, wherein the overall response rate is at least 90%.

78. The method of claim 77, wherein the overall response rate is at least 95%.

79. The method of claim 78, wherein the overall response rate is at least 99%.

80. The method of any one of claims 55-75, wherein the complete response rate is at least 65%.

81. The method of claim 80, wherein the complete response rate is at least 75%.

82. The method of claim 81, wherein the complete response rate is at least 85%.

83. The method of any one of claims 55-82, wherein the anti-CD20 monoclonal antibody is obinutuzumab or rituximab.

84. The method of any one of claims 1-54, wherein the subject is human.

85. The method of any one of claims 55-83, wherein each subject in the population is human.

86. The method of any one of claims 1-54, wherein the subject exhibits a reduction in tumor burden after being administered an effective amount of mosunetuzumab and an effective amount of lenalidomide.

87. The method of claim 86, wherein the reduction in tumor burden is determined by computed tomography (CT).

88. The method of claim 86 or 87, wherein the reduction in tumor burden is a decrease in the sum of the product of the diameters (SPD) of target lesions.

89. The method of claim 88, wherein the decrease in SPD is at least 40%.

90. The method of claim 89, wherein the decrease in SPD is at least 60%.

91. The method of claim 90, wherein the decrease in SPD is at least 80%.

92. The method of any one of claims 55-83, wherein at least 45% of subjects in the population exhibit a reduction in tumor burden after being administered an effective amount of mosunetuzumab and an effective amount of lenalidomide.

93. The method of claim 92, wherein at least 60% of subjects in the population exhibit a reduction in tumor burden after being administered an effective amount of mosunetuzumab and an effective amount of lenalidomide.

94. The method of claim 93, wherein at least 75% of subjects in the population exhibit a reduction in tumor burden after being administered an effective amount of mosunetuzumab and an effective amount of lenalidomide.

95. The method of any one of claims 92-94, wherein the reduction in tumor burden is determined by computed tomography (CT).

96. The method of any one of claims 92-95, wherein the reduction in tumor burden is a decrease in the sum of the product of the diameters (SPD) of target lesions.

97. The method of claim 96, wherein the decrease in SPD is at least 40%.

98. The method of claim 97, wherein the decrease in SPD is at least 60%.

99. The method of claim 98, wherein the decrease in SPD is at least 80%.

100. The method of any one of claims 8-54, wherein the dosing regimen further comprises administration of a corticosteroid.

101. The method of claim 100, wherein the corticosteroid is administered to the subject during the first dosing cycle.

102. The method of claim 101, wherein the first dosing cycle comprises a first dose (C1D1), a second dose (C1D2), and a third dose (C1D3) of the corticosteroid.

103. The method of claim 102, wherein the C1D1, the C1D2, and the C1D3 of the corticosteroid are administered to the subject on Days 1, 8, and 15, respectively, of the first dosing cycle.

104. The method of claim 103, wherein each single dose C1D1-C1D3 of the corticosteroid is administered to the subject before administration of the C1D1-C1D3, respectively, of mosunetuzumab.

105. The method of any one of claims 100-104, wherein the corticosteroid is administered to the subject on the first dosing cycle and not on the second dosing cycle.

106. The method of any one of claims 100-104, wherein the corticosteroid is administered to the subject on the second dosing cycle.

107. The method of claim 106, wherein the second dosing cycle comprises a single dose (C2D1) of the corticosteroid.

108. The method of claim 107, wherein the C2D1 of the corticosteroid is administered to the subject on Day 1 of the second dosing cycle.

109. The method of claim 108, wherein the C2D1 of the corticosteroid is administered to the subject before administration of the C2D1 of mosunetuzumab.

110. The method of any one of claims 55-83, wherein the dosing regimen further comprises administration of a corticosteroid.

111. The method of claim 110, wherein the corticosteroid is administered to the subjects during the first dosing cycle.

112. The method of claim 111, wherein the first dosing cycle comprises a first dose (C1D1), a second dose (C1D2), and a third dose (C1D3) of the corticosteroid.

113. The method of claim 112, wherein the C1D1, the C1D2, and the C1D3 of the corticosteroid are administered to the subjects on Days 1, 8, and 15, respectively, of the first dosing cycle.

114. The method of claim 113, wherein each single dose C1D1-C1D3 of the corticosteroid is administered to the subjects before administration of the C1D1-C1D3, respectively, of mosunetuzumab.

115. The method of any one of claims 110-114, wherein the corticosteroid is administered to the subjects on the first dosing cycle and not on the second dosing cycle.

116. The method of any one of claims 110-115, wherein the corticosteroid is administered to the subjects on the second dosing cycle.

117. The method of claim 116, wherein the second dosing cycle comprises a single dose (C2D1) of the corticosteroid.

118. The method of claim 117, wherein the C2D1 of the corticosteroid is administered to the subjects on Day 1 of the second dosing cycle.

119. The method of claim 118, wherein the C2D1 of the corticosteroid is administered to the subjects before administration of the C2D1 of mosunetuzumab.

120. The method of claim 22 or 23, wherein each additional dosing cycle comprises administering to the subject an additional dose of the corticosteroid.

121. The method of claim 120, wherein each additional dose of the corticosteroid is administered on Day 1 of each additional dosing cycle.

122. The method of claim 121, wherein each additional dose of the corticosteroid is administered to the subject before administration of each additional dose of mosunetuzumab.

123. The method of any one of claims 100-122, wherein the corticosteroid is administered intravenously.

124. The method of any one of claims 100-123, wherein the corticosteroid is dexamethasone.

125. The method of claim 124, wherein each dose of dexamethasone is 10 mg.
